(19) United States
(12) Patent Application Publication
(10) Pub. No.: US 2025/0050019 A1
(43) Pub. Date: Feb. 13, 2025

(54) SYSTEMS, DEVICES, AND METHODS FOR ANALYTE MONITORING

(71) Applicant: ABBOTT DIABETES CARE INC., Alameda, CA (US)

(72) Inventor: TIMOTHY C. DUNN, San Francisco, CA (US)

(73) Assignee: ABBOTT DIABETES CARE INC., Alameda, CA (US)

(21) Appl. No.: 18/710,049

(22) PCT Filed: Nov. 14, 2022
(86) PCT No.: PCT/US2022/049824

§ 371 Date: May 14, 2024

Related U.S. Application Data
(60) Provisional application No. 63/279,509, filed on Nov. 15, 2021.

(51) Int. Cl. A61M 5/172; G16H 10/60; G16H 50/30

(52) U.S. Cl. CPC A61M 5/1723; G16H 10/60; G16H 50/30; A61M 2202/0486; A61M 2230/201

(57) ABSTRACT

A glucose monitoring system comprising a sensor control device comprising an analyte sensor coupled with sensor electronics, the sensor control device configured to transmit data indicative of an analyte level of a subject, and a reader device. The reader device comprises a wireless communication circuitry configured to receive the data indicative of the analyte level and a glycated hemoglobin level for the subject, a non-transitory memory, and at least one processor communicatively coupled to the non-transitory memory and the analyte sensor and configured: calculate a plurality of personalized glucose metrics for the subject using at least one physiological parameter and at least one of the received data indicative of the analyte level or the received glycated hemoglobin level, and a display, on a display of the reader device, a report comprising a plurality of interfaces including at least two or more of the received data indicative of the analyte level, the received glycated hemoglobin level, or the calculated plurality of personalized glucose metrics, wherein the plurality of interfaces comprising the report are based on a user type.

### CROSS-REFERENCE TO RELATED APPLICATIONS

This application claims the benefit, under 35 U.S.C. § 119(e), of U.S. Provisional Patent Application No. 63/279,509, filed Nov. 15, 2021, which is incorporated herein by reference in its entirety and for all purposes.

### FIELD

The subject matter described herein relates generally to improved analyte monitoring systems, as well as methods and devices relating thereto.

### BACKGROUND

The detection and/or monitoring of analyte levels, such as glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, uric acid, etc., or the like, can be important to the health of an individual having diabetes. Patients suffering from diabetes mellitus can experience complications including loss of consciousness, cardiovascular disease, retinopathy, neuropathy, and nephropathy. Diabetics are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device may be worn on the body of an individual who requires analyte monitoring. To increase comfort and convenience for the individual, the sensor control device may have a small form-factor and can be applied by the individual with a sensor applicator. The application process includes inserting at least a portion of a sensor that senses a user's analyte level in a bodily fluid located in a layer of the human body, using an applicator or insertion mechanism, such that the sensor comes into contact with a bodily fluid. The sensor control device may also be configured to transmit analyte data to another device, from which the individual, her health care provider (“HCP”), or a caregiver can review the data and make therapy decisions.

Despite their advantages, however, some people are reluctant to use analyte monitoring systems for various reasons, including the complexity and volume of data presented, a learning curve associated with the software and user interfaces for analyte monitoring systems, and an overall paucity of actionable information presented.

Thus, needs exist for improved digital and graphical user interfaces for analyte monitoring systems, as well as methods and devices relating thereto, that are robust, user-friendly, and provide for timely and actionable responses.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

The achieve these and other advantages and in accordance with the purpose of the disclosed subject matter, as embodied and broadly described, the disclosed subject matter is directed to systems monitoring glucose. According to an embodiment, a system for monitoring glucose can include a sensor control device and a reader device. The sensor control device can include an analyte sensor coupled with sensor electronics and can be configured to transmit data indicative of an analyte level of a subject. The reader device can include a wireless communication circuitry configured to receive the data indicative of the analyte level and a glycated hemoglobin level for the subject, a non-transitory memory, at least one processor communicatively coupled to the non-transitory memory and the analyte sensor and configured to calculate a plurality of personalized glucose metrics for the subject using at least one physiological parameter and at least one of the received data indicative of the analyte level or the received glycated hemoglobin level, and display, on a display of the reader device, a report comprising a plurality of interfaces including at least two or more of the received data indicative of the analyte level, the received glycated hemoglobin level, or the calculated plurality of personalized glucose metrics, wherein the plurality of interfaces comprising the report are based on a user type.

As embodied herein, the plurality of personalized glucose metrics can include one or more of an adjusted A1c or personalized A1c, a calculated A1c, an adjusted calculated A1c, a personalized glucose, a personalized average glucose, or a personalized time in range. Further, the at least one processor can be configured to calculate a plurality of personalized glucose targets corresponding to the calculated plurality of personalized glucose metrics. The plurality of interfaces can further include the plurality of personalized glucose targets. Additionally, the plurality of personalized glucose targets can include one or more of a target glucose range or a target average glucose. As embodied herein, the personalized target glucose range can include a personalized lower glucose limit. Alternatively, the personalized target glucose range can include a personalized upper glucose limit.

As embodied herein, the at least one physiological parameter can be selected from the group consisting of: a red blood cell glucose uptake, a red blood cell lifespan, a red blood cell glycation rate constant, a red blood cell generation rate constant, a red blood cell elimination constant, and an apparent glycation constant. Further, the plurality of interfaces can include the at least one physiological parameter for the subject.

As embodied herein, the user type can include a health care professional. Further, the plurality of interfaces can include a glucose monitoring data interface, a glycated hemoglobin interface, a personalized a1c interface, a personalized glucose interface, a personalized average glucose, and a personalized time in range interface.

As embodied herein, the user type can include the subject. Further, the plurality of interfaces can include a glucose monitoring data interface, a glycated hemoglobin interface, a mean glucose interface, and a time in range interface.

As embodied herein, the plurality of interfaces comprising the report can be predetermined based on the user type.

As embodied herein, the plurality of interfaces comprising the report can be selected by the user.

As embodied herein, the at least one processor can be further configured to output a notification if at least one of the plurality of personalized glucose metrics is at or above the corresponding plurality of personalized glucose targets. As embodied herein, the notification can be a visual notification. Alternatively, the notification can be an audio notification. The notification can also be an alarm. As embodied herein, the notification can be a prompt.

As embodied herein, the reader device can wirelessly receive the glycated hemoglobin level for the subject from an electronic medical records system.

As embodied herein, the reader device can wirelessly receive the glycated hemoglobin level for the subject from a cloud-based database.

As embodied herein, the reader device can wirelessly receive the glycated hemoglobin level for the subject from a QR code.

As embodied herein, the reader device can wirelessly receive the glycated hemoglobin level for the subject from a home test kit.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of an analyte monitoring system comprising a sensor applicator, a sensor control device, a reader device, a network, a trusted computer system, and a local computer system.

FIG. 2A is a block diagram depicting an example embodiment of a reader device.

FIGS. 2B and 2C are block diagrams depicting example embodiments of sensor control devices.

FIGS. 2D to 2I are example embodiments of GUIs comprising sensor results interfaces.

FIGS. 2J-L are example embodiments of GUIs comprising glucose monitoring data interface and calculated A1c interfaces.

FIGS. 3A to 3F are example embodiments of GUIs comprising time-in-ranges interfaces.

FIGS. 4A to 40 are example embodiments of GUIs comprising analyte level and trend alert interfaces.

FIGS. 5A and 5B are example embodiments of GUIs comprising sensor usage interfaces.

FIGS. 5C to 5F are example embodiments of report GUIs including sensor usage information.

FIGS. 5G-5L are example embodiments of GUIs relating to an analyte monitoring software application.

FIGS. 6A and 6B are flow diagrams depicting example embodiments of methods for data backfilling in an analyte monitoring system.

FIG. 6C is a flow diagram depicting an example embodiment of a method for aggregating disconnect and reconnect events in an analyte monitoring system.

FIG. 7 is a flow diagram depicting an example embodiment of a method for failed or expired sensor transmissions in an analyte monitoring system.

FIGS. 8A and 8B are flow diagrams depicting example embodiments of methods for data merging in an analyte monitoring system.

FIGS. 8C to 8E are graphs depicting data at various stages of processing according to an example embodiment of a method for data merging in an analyte monitoring system.

FIG. 9A is a flow diagram depicting an example embodiment of a method for sensor transitioning in an analyte monitoring system.

FIGS. 9B to 9D are example embodiments of GUIs to be displayed according to an example embodiment of a method for sensor transitioning in an analyte monitoring system.

FIG. 10A is a flow diagram depicting an example embodiment of a method for generating a sensor insertion failure system alarm.

FIGS. 10B to 10D are example embodiments of GUIs to be displayed according to an example embodiment of a method for generating a sensor insertion failure system alarm.

FIG. 11A is a flow diagram depicting an example embodiment of a method for generating a sensor termination system alarm.

FIGS. 11B to 11D are example embodiments of GUIs to be displayed according to an example embodiment of a method for generating a sensor termination system alarm.

FIG. 12 illustrates an example timeline 100 illustrating collection of at least one HbA1c value and a plurality of glucose levels for a time period.

FIG. 13 illustrates an example of a physiological parameter analysis system for providing physiological parameter analysis in accordance with some of the embodiments of the present disclosure.

FIG. 14 illustrates an example of a physiological parameter analysis system for providing physiological parameter analysis in accordance with some of the embodiments of the present disclosure.

FIG. 15 illustrates an example of a calculated HbA1c (eHbA1c) report that may be generated as an output by a physiological parameter analysis system in accordance with some of the embodiments of the present disclosure.

FIG. 16A illustrates an example of a method of determining a personalized-target glucose range in accordance with some of the embodiments of the present disclosure.

FIG. 16B illustrates an example of a personalized-target glucose range report that may be generated as an output by a physiological parameter analysis system in accordance with some of the embodiments of the present disclosure.

FIG. 17 illustrates an example of a personalized-target average glucose report that may be generated as an output by a physiological parameter analysis system in accordance with some of the embodiments of the present disclosure.

FIGS. 18A-C illustrate a comparison between the laboratory HbA1c levels at day 200 (±5 days) relative to the estimated HbA1c (eHbA1c) values for two different models (18A and 18B) and calculated HbA1c (cHbA1c) values for the kinetic model of the present disclosure (18C).

FIG. 19 illustrates an example study subject's data with the measured glucose levels (solid line), laboratory HbA1c readings (open circles), cHbA1c model values (long dashed line), and 14-day eHbA1c model values (dotted line).

FIG. 20 illustrates the relationship between steady glucose and equilibrium HbA1c (1) as determined using the standard conversion of HbA1c to estimated average glucose (dashed line with error bars) and (2) as measured for the 90 participants (solid lines).

FIG. 21 illustrates the relationship between K (dL/mg) and mean glucose level target (mg/dl) for varying HbA1c target values using the kinetic model of the present disclosure.

FIG. 22 is a graphical representation of mean glucose and laboratory A1c.

FIGS. 23-29 provide case examples embodiments of reports of the present disclosure.

FIG. 30 illustrates an exemplary Health Care Provider interface in accordance with embodiments of the present disclosure.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of this disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of this application. Nothing herein is to be construed as an admission that this disclosure is not entitled to antedate such publication by virtue of prior disclosure. Further, the dates of publication provided may be different from the actual publication dates which may need to be independently confirmed.

Generally, embodiments of this disclosure include GUIs and digital interfaces for analyte monitoring systems, and methods and devices relating thereto. Accordingly, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely noninvasive.

Furthermore, for each and every embodiment of a method disclosed herein, systems and devices capable of performing each of those embodiments are covered within the scope of this disclosure. For example, embodiments of sensor control devices, reader devices, local computer systems, and trusted computer systems are disclosed, and these devices and systems can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps.

As previously described, a number of embodiments described herein provide for improved GUIs for analyte monitoring systems, wherein the GUIs are highly intuitive, user-friendly, and provide for rapid access to physiological information of a user. According to some embodiments, a Time-in-Ranges GUI of an analyte monitoring system is provided, wherein the Time-in-Ranges GUI comprises a plurality of bars or bar portions, wherein each bar or bar portion indicates an amount of time that a user's analyte level is within a predefined analyte range correlating with the bar or bar portion. According to another embodiment, an Analyte Level/Trend Alert GUI of an analyte monitoring system is provided, wherein the Analyte Level/Trend Alert GUI comprises a visual notification (e.g., prompts, alert, alarm, pop-up window, banner notification, etc.), and wherein the visual notification includes an alarm condition, an analyte level measurement associated with the alarm condition, and a trend indicator associated with the alarm condition. In sum, these embodiments provide for a robust, user-friendly interfaces that can increase user engagement with the analyte monitoring system and provide for timely and actionable responses by the user, to name a few advantages.

In addition, a number of embodiments described herein provide for improved digital interfaces for analyte monitoring systems. According to some embodiments, improved methods, as well as systems and device relating thereto, are provided for data backfilling, aggregation of disconnection and reconnection events for wireless communication links, expired or failed sensor transmissions, merging data from multiple devices, transitioning of previously activated sensors to new reader devices, generating sensor insertion failure system alarms, and generating sensor termination system alarms. Collectively and individually, these digital interfaces improve upon the accuracy and integrity of analyte data being collected by the analyte monitoring system, the flexibility of the analyte monitoring system by allowing users to transition between different reader devices, and the alarming capabilities of the analyte monitoring system by providing for more robust inter-device communications during certain adverse conditions, to name only a few. Other improvements and advantages are provided as well. The various configurations of these devices are described in detail by way of the embodiments which are only examples.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Example Embodiment of In Vivo Analyte Monitoring System

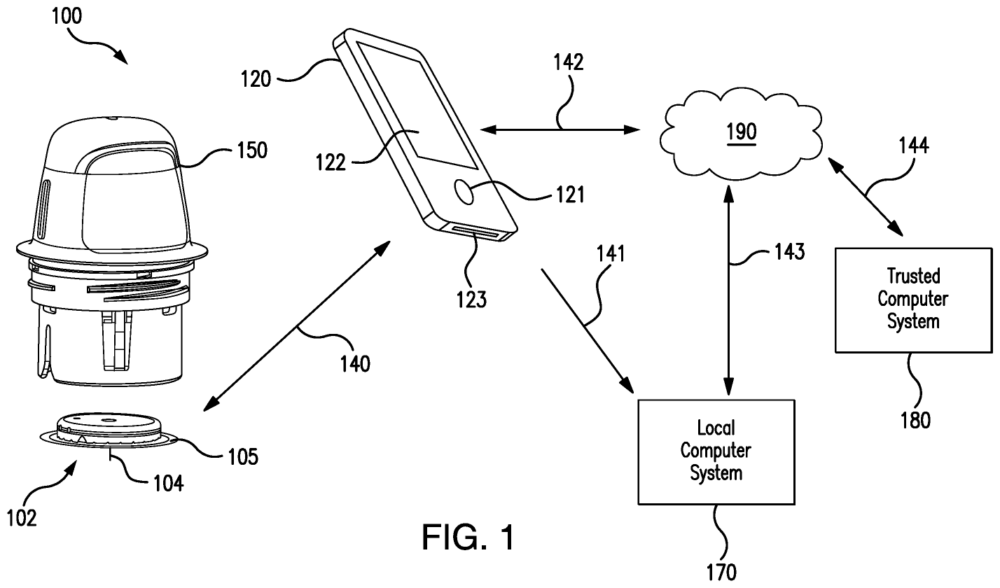

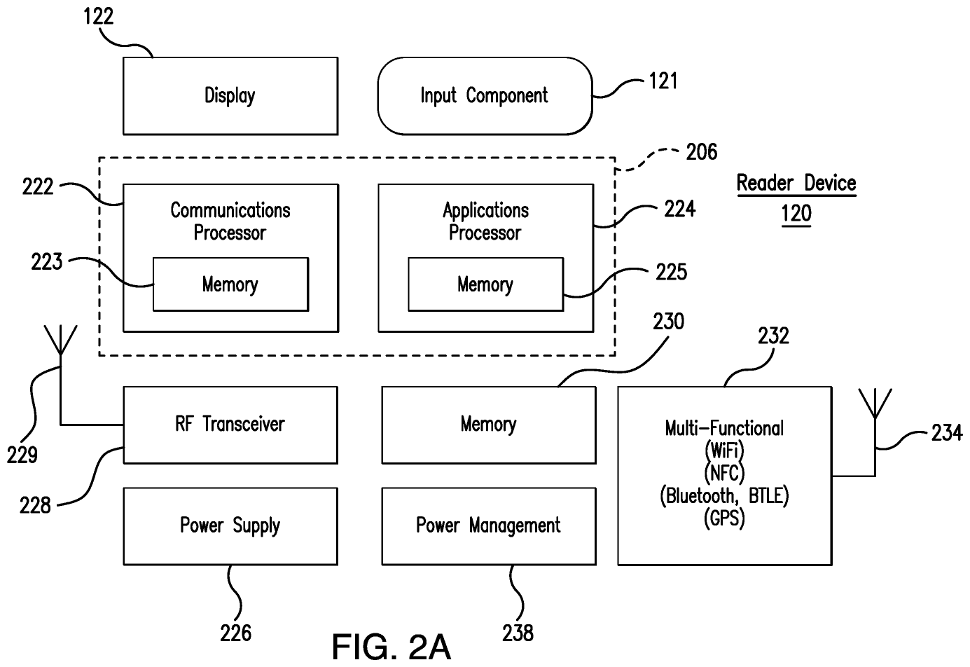

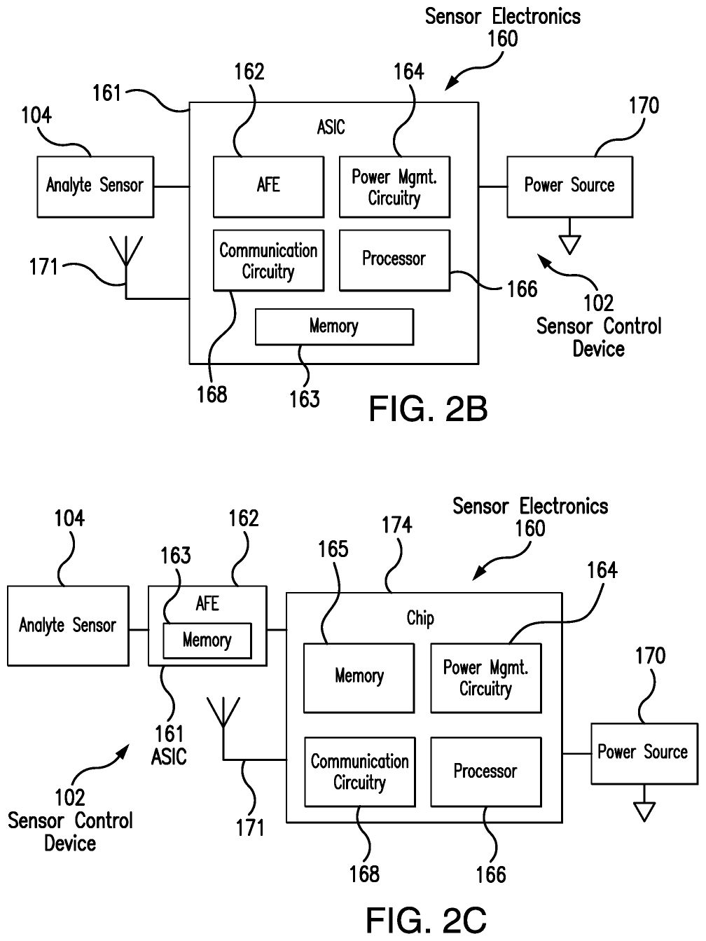

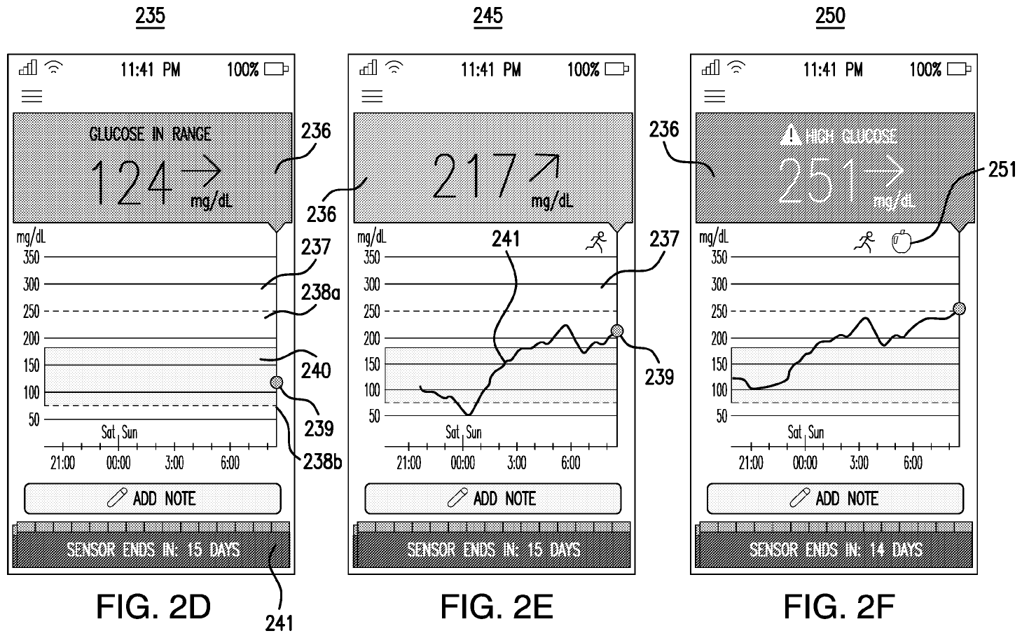

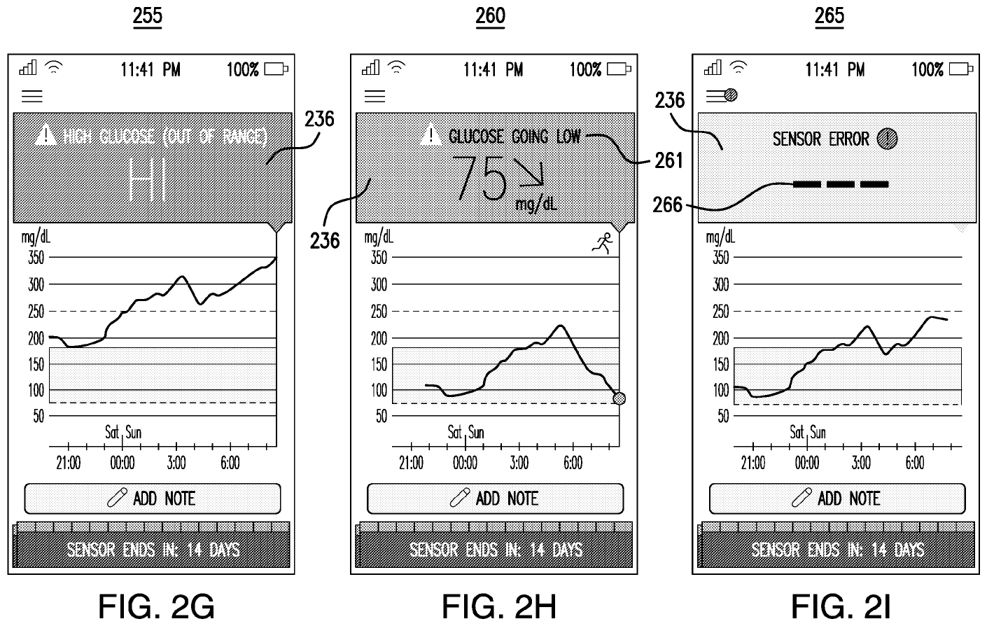

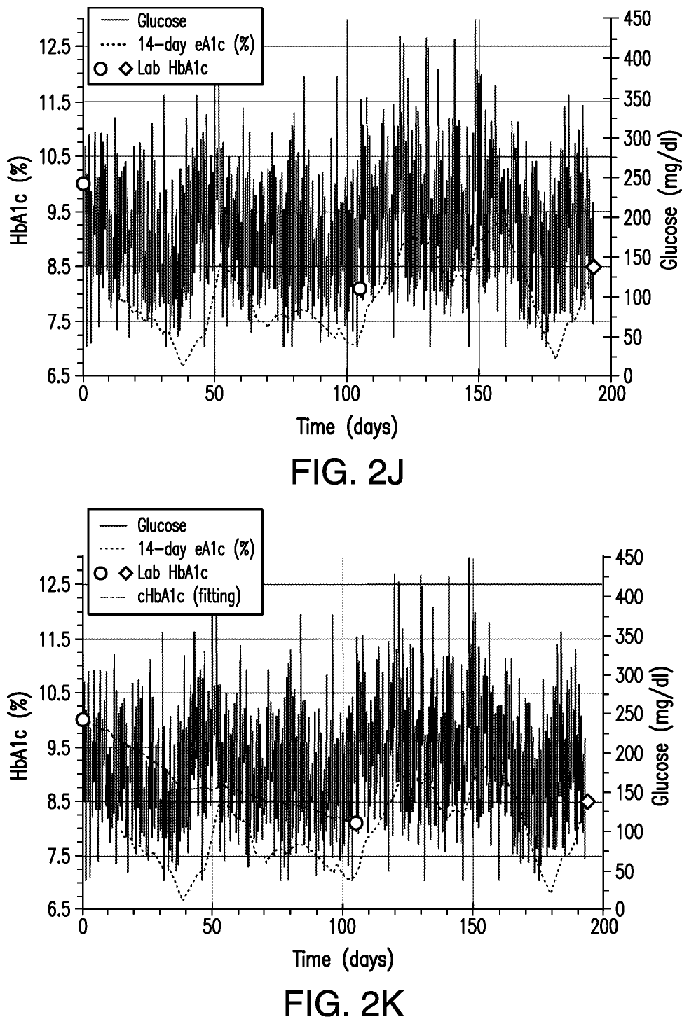

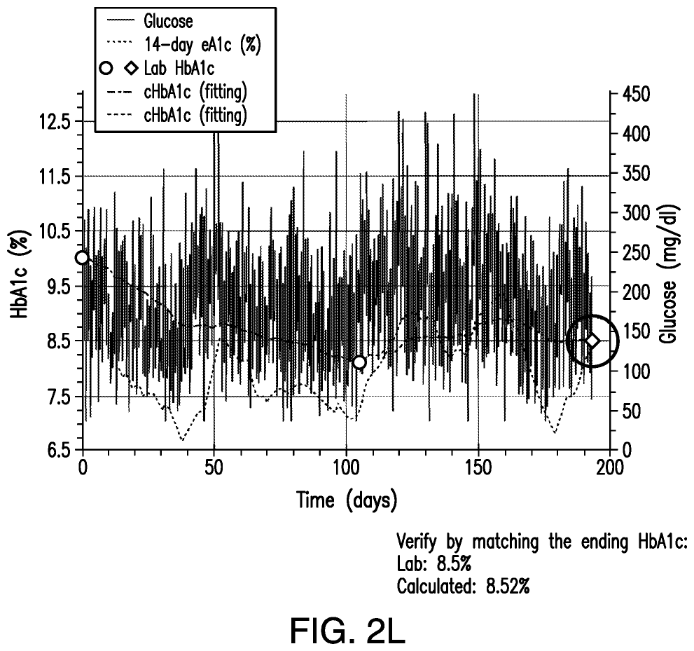

FIG. 1 is a conceptual diagram depicting an example embodiment of an analyte monitoring system 100 that includes a sensor applicator 150, a sensor control device 102, and a reader device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can view and use applications installed in memory on reader device 120 using screen 122 (which, in many embodiments, can comprise a touchscreen), and input 121. A device battery of reader device 120 can be recharged using power port 123. While only one reader device 120 is shown, sensor control device 102 can communicate with multiple reader devices 120. Each of the reader devices 120 can communicate and share data with one another. More details about reader device 120 is set forth with respect to FIG. 2A below. Reader device 120 can communicate with local computer system 170 via a communication path 141 using a wired or wireless communication protocol. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how reader device 120 can communicate via a communications path 142 with network 190, by a wired or wireless communication protocol as described previously. Network 190 can be any of a number of networks, such as private networks and public networks, local area or wide area networks, and so forth. A trusted computer system 180 can include a cloud-based platform or server, and can provide for authentication services, secured data storage (e.g., storage of analyte measurement data received from reader device), report generation, and can communicate via communications path 144 with network 190 by wired or wireless technique. In addition, although FIG. 1 depicts trusted computer system 180 and local computer system 170 communicating with a single sensor control device 102 and a single reader device 120, it will be appreciated by those of skill in the art that local computer system 170 and/or trusted computer system 180 are each capable of being in wired or wireless communication with a plurality of reader devices and sensor control devices.

Additional details of suitable analyte monitoring devices, systems, methods, components and the operation thereof along with related features are set forth in U.S. Pat. No. 9,913,600 to Taub et. al., International Publication No. WO2018/136898 to Rao et. al., International Publication No. WO2019/236850 to Thomas et. al., and U.S. Patent Publication No. 2020/01969191 to Rao et al., each of which is incorporated by reference in its entirety herein.

### Example Embodiment of Reader Device

FIG. 2A is a block diagram depicting an example embodiment of a reader device 120, which, in some embodiments, can comprise a smart phone or a smart watch. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further, reader device 120 can also include a multi-functional transceiver 232, which can comprise wireless communication circuitry, and which can be configured to communicate over Wi-Fi, NFC, Bluetooth, BTLE, and GPS with one or more antenna 234. As understood by one of skill in the art, these components are electrically and communicatively coupled in a manner to make a functional device.

### Example Embodiments of Sensor Control Devices

FIGS. 2B and 2C are block diagrams depicting example embodiments of sensor control devices 102 having analyte sensors 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 170, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data. According to some embodiments, for example, a current glucose value can be transmitted from sensor control device 102 to reader device 120 every minute, and historical glucose values can be transmitted from sensor control device 102 to reader device 120 every five minutes.

In some embodiments, to conserve power and processing resources on sensor control device 102, digital data received from AFE 162 can be sent to reader device 120 (not shown) with minimal or no processing. In still other embodiments, processor 166 can be configured to generate certain predetermined data types (e.g., current glucose value, historical glucose values) either for storage in memory 163 or transmission to reader device 120 (not shown), and to ascertain certain alarm conditions (e.g., sensor fault conditions), while other processing and alarm functions (e.g., high/low glucose threshold alarms) can be performed on reader device 120. Those of skill in the art will understand that the methods, functions, and interfaces described herein can be performed—in whole or in part—by processing circuitry on sensor control device 102, reader device 120, local computer system 170, or trusted computer system 180.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 may include memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Example Embodiments of Graphical User Interfaces for Analyte Monitoring Systems

Described herein are example embodiments of GUIs for analyte monitoring systems. As an initial matter, it will be understood by those of skill in the art that the GUIs described herein comprise instructions stored in a memory of reader device 120, local computer system 170, trusted computer system 180, and/or any other device or system that is part of, or in communication with, analyte monitoring system 100. These instructions, when executed by one or more processors of the reader device 120, local computer system 170, trusted computer system 180, or other device or system of analyte monitoring system 100, cause the one or more processors to perform the method steps and/or output the GUIs described herein. Those of skill in the art will further recognize that the GUIs described herein can be stored as instructions in the memory of a single centralized device or, in the alternative, can be distributed across multiple discrete devices in geographically dispersed locations.

### Example Embodiments of Models for Personalized Glucose-Related Metrics

Described herein are example embodiments of exemplary embodiments of models for personalized glucose-related metrics. The present disclosure generally describes methods, devices, and systems for determining physiological parameters related to the kinetics of red blood cell glycation, elimination, and generation and reticulocyte maturation within the body of a subject. Such physiological parameters can be used, for example, to calculate a more reliable calculated HbA1c (cHbA1c), adjusted or personalized HbA1c (aHbA1c), adjusted calculated HbA1c (acHbA1c), and/or a personalized target glucose range, among other things, for subject-personalized diagnoses, treatments, and/or monitoring protocols.

Herein, the terms “HbA1c level,” “HbA1c value,” and “HbA1c” are used interchangeably. Herein, the terms “personalized A1c,” “personalized HbA1c,” “aHbA1c level,” “aHbA1c value,” and “aHbA1c” are used interchangeably. Herein, the terms “cHbA1c level,” “cHbA1c value,” “cHbA1c,” and “GD-Ale” are used interchangeably and/or a personalized target glucose range, among other things. Herein, the terms “acHbA1c level,” “acHbA1c value,” and “acHbA1c,” are used interchangeably.

### Kinetic Model

High glucose exposure in specific organs (particularly eye, kidney and nerve) is a critical factor for the development of diabetes complications. A laboratory HbA1c (also referred to in the art as a measured HbA1c) is routinely used to assess glycemic control, but studies report a disconnect between this glycemic marker and diabetes complications in some individuals. The exact mechanisms for the failure of laboratory HbA1c to predict diabetes complications are not often clear but likely in some cases to be related to inaccurate estimation of intracellular glucose exposure in the affected organs.

Formula 1 illustrates the kinetics of red blood cell hemoglobin glycation (or referred to herein simply as red blood cell glycation), red blood cell elimination, and red blood cell generation, where “G” is free glucose, “R” is a non-glycated red blood cell, and “GR” is glycated red blood cell hemoglobin. The rate at which glycated red blood cell hemoglobin (GR) are formed is referred to herein as a red blood cell hemoglobin glycation rate constant (k_{gly} typically having units of dl_*mg⁻¹*day¹).

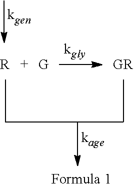

Over time, red blood cells including the glycated red blood cells are continuously eliminated from a subject's circulatory system and new red blood cells are generated, typically at a rate of approximately 2 million cells per second. The rates associated with elimination and generation are referred to herein as a red blood cell elimination constant (k_{age} typically having units of day⁻¹) and a red blood cell generation rate constant (k_{gen} typically having units of M²/day), respectively. Since the amount of red blood cells in the body is maintained at a stable level most of time, the ratio of k_{age} and k_{gen} should be an individual constant that is the square of red blood cell concentration.

Relative to glycation, Formula 2 illustrates the mechanism in more detail where glucose transporter 1 (GLUT1) facilitates glucose (G) transport into the red blood cell. Then, the intracellular glucose (GI) interacts with the hemoglobin (Hb) to produce glycated hemoglobin (HbG) where the hemoglobin glycation reaction rate constant is represented by k_{g} (typically having units of dl_*mg⁻¹*day¹). A typical experiment measured k_{g} value is 1.2×10³ db/mg/day. Hemoglobin glycation reaction is a multi-step non-enzymatic chemical reaction, therefore k_{g} should be a universal constant. The rate constant for the glucose to be transported into the red blood cell and glycated the Hb into HbG is kgly. Then, k_{age} describes red blood cell elimination (along with hemoglobin), also described herein as the red blood cell turnover rate.

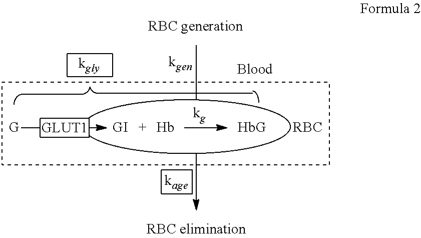

While raised intracellular glucose is responsible for diabetes complications, extracellular hyperglycemia selectively damages cells with limited ability to adjust cross-membrane glucose transport effectively. HbA1c has been used as a biomarker for diabetes-related intracellular hyperglycemia for two main reasons. First, the glycation reaction occurs within red blood cells (RBCs) and therefore HbA1c is modulated by intracellular glucose level. Second, RBCs do not have the capacity to adjust glucose transporter GLUT1 levels and thus are unable to modify cross-membrane glucose uptake, behaving similarly to cells that are selectively damaged by extracellular hyperglycemia. Therefore, under conditions of fixed RBC lifespan and cross-membrane glucose uptake, HbA1c mirrors intracellular glucose exposure in organs affected by diabetes complications. However, given the inter-individual variability in both cross-membrane glucose uptake and RBC lifespan, laboratory HbA1c may not always reflect intracellular glucose exposure. While variation in RBC cross-membrane glucose uptake is likely to be relevant to the risk of estimating diabetes complications in susceptible organs, red blood cell lifespan is unique to RBCs and therefore irrelevant to the complication risk in other tissues. This explains the inability to clinically rely on laboratory HbA1c in those with hematological disorders characterized by abnormal RBC turnover and represents a possible explanation for the apparent “disconnect” between laboratory HbA1c and development of complications in some individuals with diabetes (FIG. 1).

To overcome the limitations of laboratory HbA1c, a measure of personalized HbA1c has been developed, which takes into account individual variations in both RBC turnover and cellular glucose uptake. The current work aims to extend this model by adjusting for a standard RBC lifespan of 100 days (equivalent to RBC turnover rate of 1% per day, or mean RBC age of 50 days) to establish a new clinical marker, which we term adjusted HbA1c (aHbA1c). We propose that aHbA1c is the most relevant glycemic marker for estimating organ exposure to hyperglycemia and risk of future diabetes-related complications

As described previously, HbA1c is a commonly used analyte indicative of the fraction of the glycated hemoglobin found in red blood cells. Therefore, a kinetic model can be used, for example, to derive a calculated HbA1c based on at least the glucose levels measured for a subject. However, the kinetic model can also be applied to HbA1. For simplicity, HbA1c is uniformly used herein, but HbA1 could be substituted except in instances where specific HbA1c values are used (e.g., see Equations 15 and 16). In such instances, specific HbA1 values could be used to derive similar equations.

Typically, when kinetically modeling physiological processes, assumptions are made to focus on the factors that affect the physiological process the most and simplify some of the math.

The present disclosure uses only the following set of assumptions to kinetically model the physiological process illustrated in Formula 1. First, glucose concentration is high enough not to be affected by the red blood cell glycation reaction. Second, there is an absence of abnormal red blood cells that would affect HbA1c measurement, so the hematocrit is constant for the period of interest. This assumption was made to exclude extreme conditions or life events that are not normally present and may adversely affect the accuracy of the model. Third, the glycation process has first order dependencies on both red blood cell and glucose concentrations. Fourth, newly-generated red blood cells have a negligible amount of glycated hemoglobin, based on previous reports that reticulocyte HbA1c is very low and almost undetectable. Fifth, red blood cell production inversely correlates with total cellular concentration, whereas elimination is a first order process.

With the five assumptions described above for this kinetic model, the rate of change in glycated and non-glycated red blood cells can be modeled by differential Equations 1 and 2.

$$d [ GR ∨ dt = kgly [ G ] [ R ] - {k}_{age} [ GR ] \qquad Equation 1$$

$$( d [ R ] ) / dt = {k}_{gen} / C - {k}_{age} [ R ] - {k}_{gly} [ G ] [ R ] \qquad Equation 2$$

C is the whole population of red blood cells, where C=[ff]+[GR](Equation 2a). C typically has units of M (mol/L), [R] and [GR] typically have units of M, and [G] typically has units of mg/dl_.

Assuming a steady state, where the glucose level is constant and the glycated and non-glycated red blood cell concentrations remain stable (d[GR]/dt=(d[R])/dt=0), the following two equations can be derived. Equation 3 defines the apparent glycation constant K (typically with units of dL/mg) as the ratio of k_{gly} and k_{age}, whereas Equation 4 establishes the dependency between red blood cell generation and elimination rates.

$$K = {k}_{gly} / {k}_{age} = [ GR ] / [ G ] [ R ] \qquad Equation 3$$

$${k}_{gen} / {k}_{age} = {C}^{2} \qquad Equation 4$$

For simplicity, k_{age} is used hereafter to describe the methods, devices, and systems of the present disclosure. Unless otherwise specified, k_{gen} can be substituted for k_{age}. To substitute k_{gen} for k_{age}, Equation 4 would be rearranged to k_{gen}−k_{age}*C.

HbA1c is the fraction of glycated hemoglobin as shown in Equation 5.

$$HbAlc = [ GR ] / C = ( C - [ R ] ) / C \qquad Equation 5$$

In a hypothetical state when a person infinitely holds the same glucose level, HbA1c in Equation 5 can be defined as “equilibrium HbA1c” (EA) (typically reported as a % (e.g., 6.5%) but used in decimal form (e.g., 0.065) in the calculations). For a given glucose level, EA (Equation 6) can be derived from Equations 2a, 3, and 5.

$$EA = ( {k}_{gly} [ G ] ) / ( {k}_{age} + {k}_{gly} [ G ] ) = [ G ] / ( {K}^{- 1} + [ G ] ) \qquad Equation 6$$

EA is an estimate of HbA1c based on a constant glucose concentration [G] for a long period. This relationship effectively approximates the average glucose and HbA1c for an individual having a stable day-to-day glucose profile. EA depends on K, the value of which is characteristic to each subject. Equation 6 indicates that the steady glucose is not linearly correlated with EA. Steady glucose and EA may be approximated with a linear function within a specific range of glucose level, but not across the full typical clinical range of HbA1c. Furthermore, in real life with continuous fluctuations of glucose levels, there is no reliable linear relationship between laboratory HbA1c and average glucose for an individual.

Others have concluded this also and produced kinetic models to correlate a measured HbA1c value to average glucose levels. For example, The American Diabetes Association has an online calculator for converting HbA1c values to estimated average glucose levels. However, this model is based on an assumption that k_{age} and k_{gly} do not substantially vary between subjects, which is illustrated to be false in Example 1 below. Therefore, the model currently adopted by the American Diabetes Association considers k_{age} and k_{gly} as constants and not variable by subject.

A more recent model by Higgens et al. (Sci. Transl. Med. 8, 359ral30, 2016) has been developed that removed the assumption that red blood cell life is constant. However, the more recent model still assumes that k_{gly} does not substantially vary between subjects.

In contrast, both k_{age} and k_{gly} are variables for the kinetic models described herein. Further, a subject's k_{gly} is used in some embodiments to derive personalized parameters relating to the subject's diabetic condition and treatment (e.g., a medication dosage, a supplement dosage, an exercise plan, a diet/meal plan, and the like).

Continuing with the kinetic model of the present disclosure, the HbA1c value (HbA1cₜ) at the end of a time period t (Equation 7) can be derived from Equation 1, given a starting HbA1c (HbA1co) and assuming a constant glucose level [G] during the time period.

$$HbA 1 {c}_{t} = EA + ( HbA 1 {c}_{0} - EA ) * {e}^{- ( {k}_{gly} [ G ] + {k}_{age} ) t} \qquad Equation 7$$

To accommodate changing glucose levels over time, each individual's glucose history is approximated as a series of time intervals t, with corresponding average glucose levels [G,]. Applying Equation 7 recursively, HbAlC_{z} at the end of time interval t_{z} can be expressed by Equation 8 for numerical calculations.

$$HbA 1 {c}_{z} = {EA}_{z} ( 1 - {D}_{z} ) + {\sum }_{i = 1}^{z - 1} [ {EA}_{i} ( 1 - {D}_{i} ) {\prod }_{j = i + 1}^{z} {D}_{j} ] + HbA 1 {c}_{0} {\prod }_{j = 1}^{z} {D}_{j}$$

Equation 8 where the decay term Dₜ=ₑ₋(y^{[G;]+/c}agₑ)^{t}£ (Equation 8a).

When solving for k_{age} and k_{gly} using Equations 6, 7, or 8, k_{age} and k_{gly} may be bounded to reasonable physiological limits, by way of nonlimiting example, of 5.0*10⁶ dl_*mg\hat{ }day⁻¹<k_{gly}<8.0*10⁶ dl_*mg\hat{ }day¹ and 0.006 day¹<k_{age}<0.024 day⁻¹. Additionally or alternatively, an empirical approach using the Broyden-Fletcher-Goldfarb-Shanno algorithm can be used with estimated initial values for k_{gly} and k_{age} (e.g., k_{gly}=4.4*10⁻⁶ dl_*mg\hat{ }day¹ and k_{age}=0.0092 day^{x}). The more glucose level data points and measured HbA1c data points, the more accurate the physiological parameters described herein are.

The value for time interval t, can be selected (e.g., by a user or developer, or by software instructions being executed on one or more processors) based on a number of factors that can vary between embodiments and, as such, the value of time interval t may vary. One such factor is the duration of time from one glucose data value (e.g., a measured glucose level at a discrete time, a value representative of glucose level for a particular time period across multiple discrete times, or otherwise) to another within the individual's glucose history. That duration of time between glucose data values can be referred to as time interval t_{g}. Time interval t_{g} can vary across the individual's glucose history such that a single glucose history can have a number of different values for time interval t_{g}. Numerous example embodiments leading to different values of time interval t_{g} are described herein. In some embodiments of glucose monitoring systems, glucose data points are determined after a fixed time interval t_{g} (e.g., every minute, every ten minutes, every fifteen minutes, etc.) and the resulting glucose history is a series of glucose data points with each point representing the glucose at the expiration of or across the fixed time interval t_{g} (e.g., a series of glucose data points at one minute intervals, etc.). [0037] In other embodiments, glucose data points are taken or determined at multiple different fixed time intervals t_{g}. For example, in some flash analyte monitoring systems (described in further detail herein), a user may request glucose data from a device (e.g., a sensor control device) that stores glucose data within a recent time period (e.g., the most recent fifteen minutes, the most recent hour, etc.) at a first relatively shorter time interval t_{g} (e.g., every minute, every two minutes), and all other data (in some cases up to a maximum of eight hours, twelve hours, twenty-four hours, etc.) outside of that recent time period is stored at a second relatively longer time interval t_{g} (e.g., every ten minutes, every fifteen minutes, every twenty minutes, etc.). The data stored at the second, relatively longer time interval can be determined from data originally taken at the relatively shorter time interval t_{g} (e.g., an average, median, or other algorithmically determined value). In such an example the resulting glucose history is dependent on how often a user requests glucose data, and can be a combination of some glucose data points at the first time interval t_{g} and others at the second time interval t_{g}. Of course, more complex variations are also possible with, for example, three or more time intervals t_{g}. In some embodiments, glucose data collected with ad hoc adjunctive measurements (e.g., a finger stick and test strip) can also be present, which can result in even more variations of time interval t_{g}.

An example analysis performed on glucose histories for a sample of subjects (approximately 400) where glucose data points were generally present at time intervals t_{g} of one to fifteen minutes, indicated that a value for time interval t, within the range of three hours (or about three hours) to twenty four hours (or about twenty four hours) could be selected without significant loss of accuracy. Generally, shorter time intervals t, resulted in higher accuracy than longer ones, and time interval t, values closer to three hours were the most accurate. Time interval t, values less than three hours may begin to exhibit loss of accuracy due to numerical rounding errors. These rounding errors can be reduced by using longer digit strings at the expense of processing load and computing time. It should be noted that other values of time interval t, outside of the range of 3 to 24 hours may be suitable depending on the desired accuracy levels and other factors, such as the average time interval t_{g} between glucose data points.

Another factor in selection of time interval t, is the existence of gaps, or missing data, in the individual's glucose history, where the gaps are longer or significantly longer than the longest time interval t_{g}. The existence of one or more such gaps can potentially lead to results bias. These gaps can result, for example, from the inability to collect glucose data across a certain time period (e.g., the user was not wearing a sensor, the user forgot to scan the sensor for data, a fault occurred, etc.). The presence of gaps and their duration should be considered in selecting time interval t. Generally, the number and duration of gaps should be minimized (or eliminated) where possible. But since gaps of this type are often difficult to eliminate, to the extent such gaps exist, in many embodiments the selection of time interval t, should be at least twice the duration of the largest (maximum) gap between glucose data points. For example, if time interval t, is selected to be 3 hours, then the maximum gap should be no longer than 90 minutes, if time interval t, is selected to be 24 hours, then the largest gap should be no longer than 12 hours, and so forth.

The value HbA1c_{z} is the estimated HbA1c of the present kinetic model, which is referred to herein as cHbA1c (calculated HbA1c) to distinguish from other eHbA1c described herein.e

As described previously and illustrated in Equation 8, EA, and D, are both affected by glucose level [G,], k_{gly}, and k_{age}. In addition, D, depends on the length of the time interval t. Equation 8 is the recursive form of Equation 7. Equations 7 and 8 describe the relationship among HbA1c, glucose level, and individual red blood cell kinetic constants k_{gly} and k_{age}.

k_{age} can be directly measured through expensive and laborious methods. Herein, the kinetic model is extended to incorporate reticulocyte maturation as a method for estimating k_{age}.

Reticulocytes are immature red blood cells and typically account for about 1% of the total red blood cells. The rate at which reticulocytes mature into mature red blood cells is kₘₐₜ (typically having units of day⁻¹). The maturation half-life for a normal reticulocyte is about 4.8 hours, which provides for Equation 9.

$${k}_{mat} = ln 2 / ( 4.8 hours ) = 3.47 {day}^{- 1} \qquad Equation 9$$

The kinetic model makes two assumptions: (1) all red blood cells are reticulocytes at time 0 and (2) reticulocytes are not eliminated (that is, reticulocytes mature to mature red blood cells and do not die). The probability density of reticulocyte age (PRET) can be represented by Equation 10.

$${p}_{RET} ( τ ) = ( {k}_{age} / ( 1 - ln 2 ) ) * {e}^{- {k}_{mat} * τ} \\ where t is the cell age . \qquad Equation 10$$

A reticulocyte production index (RPI), also known as a corrected reticulocyte count (CRC), is the percentage of total red blood cells that are reticulocytes. Therefore, RPI is the integral of PRET over cell age as shown in Equation 11, where RPI is the decimal form of the reported RPI (e.g., RPI reported at 2% is 0.02 in Equation 11).

$$RPI = {\int }_{0}^{∞} {p}_{RET} ( τ ) d τ = {k}_{age} / ( {k}_{mat} * ( 1 - ln 2 ) ) \qquad Equation 11$$

Assuming the typical kₘₐₜ is 3.47 day⁻¹, k_{age} can be estimated from a measured RPI. RPI can be determined by normal methods. For example, RPI can be determined by measuring a hematocrit percentage (HMₘ), measuring a percentage of reticulocytes (RP) in an RNA dyed blood smear, determining a maturation correction (MC) from the measured hematocrit percentage, and calculating the RPI based on Equation 12, where RP and HMₘ is used as the percentage values not the decimal form (i.e., RP reported at 3% is 3 in the equation not 0.03).

Assuming the typical kₘₐₜ is 3.47 day⁻¹, k_{age} can be estimated from a measured RPI. RPI can be determined by normal methods. For example, RPI can be determined by measuring a hematocrit percentage (HMₘ), measuring a percentage of reticulocytes (RP) in an RNA dyed blood smear, determining a maturation correction (MC) from the measured hematocrit percentage, and calculating the RPI based on Equation 12, where RP and HMₘ is used as the percentage values not the decimal form (i.e., RP reported at 3% is 3 in the equation not 0.03).

RPI=(RP*HMₘ/HMₙ)/MC Equation 12 where HMₙ is the normal hematocrit value (typically 45).

Unless otherwise specified, the typical units described are associated with their respective values. One skilled in the art would recognize other units and the proper conversions. For example, [G] is typically measured in mg/dL but could be converted to M using the molar mass of glucose. If [G] is used in M or any other variable is used with different units, the equations herein should be adjusted to account for differences in units.

Calculating Physiological Parameters from the Kinetic Model

Embodiments of the present disclosure provide kinetic modeling of red blood cell glycation, elimination, and generation and reticulocyte maturation within the body of a subject.

The physiological parameter k_{age} can be estimated from one or more RPI measurements. While k_{age} can be estimated using Equation 11 above from a single RPI measurement, two or more RPI measurements may increase the accuracy of the RPI value. Further, RPI can change over time, in response to treatment, and in response to the improvement or worsening of a disease state. Therefore, while RPI can be measured be measured in any desired intervals of time (e.g., weekly to annually), preferably RPI is measured once every three to six months.

Once k_{age} is calculated, the physiological parameters k_{gly} and/or K can be estimated from the equations described herein given at least one measured HbA1c value (also referred to as HbA1c level measurement) and a plurality of glucose levels (also referred to as glucose level measurements) over a time period immediately before the HbA1c measurement.

FIG. 12 illustrates an example time line 100 illustrating a collection of at least one measured HbA1c value 12102a, 12102b, 12102c, a plurality of glucose levels 12104a and 12104b, and at least one measured RPI value 110a, 110b, 110c over time periods 106 and 108.

The number of measured HbA1c values 12102a, 12102b, 12102c needed to calculate k_{gly} and/or K depends on the frequency and duration of the plurality of glucose levels. The number of measured RPI values 110a, 110b, 110c needed to calculate k_{age} depends on the stability of individual kₘₐt and its deviation to typical kₘₐₜ (3.47 day⁻¹). Preferably RPI is measured once every three to six months but can be measured monthly or weekly, if needed.

In a first embodiment, one measured RPI value 110b can be used to calculate k_{age}, and one measured HbA1c 12102b can be used along with the calculated k_{age} and a plurality of glucose measurements over time period 106 to calculate k_{gly} and/or K. Such embodiments are applicable to subjects with steady daily glucose measurements for a long time period 106 (e.g., over about 200 days). K may be calculated at time point 101 with Equation 6 by replacing EA with the measured HbA1c value 12102b and [G] with daily average glucose over time period 106. k_{gly} may then be calculated from Equation 3. Therefore, in this embodiment, an initial HbA1c level measurement 12102a is not necessarily required.

Because a first HbA1c value is not measured, the time interval 106 of initial glucose level measurements with frequent measurements may need to be long to obtain an accurate representation of average glucose and reduce error. Using more than 100 days of steady glucose pattern for this method may reduce error. Additional length like 200 days or more or 300 days or more further reduces error.

Embodiments where one measured HbA1c value 12102b can be used include a time period 106 about 100 days to about 300 days (or longer) with glucose levels being measured at least about 72 times per day (e.g., about every 20 minutes) to about 96 times per day (e.g., about every 15 minutes) or more often. Further, in such embodiments, the time between glucose level measurements may be somewhat consistent where an interval between two glucose level measurements should not be more than about an hour. Some missing data glucose measurements are tolerable when using only one measured HbA1c value. Increases in missing data may lead to more error.

Alternatively, in some instances where one measured HbA1c value 12102b is used, the time period 106 may be shortened if a subject has an existing glucose level monitoring history with stable, consistent glucose profile. For example, for a subject who has been testing for a prolonged time (e.g., 6 months or longer) but, perhaps, at less frequent or regimented times, the existing glucose level measurements can be used to determine and analyze a glucose profile. Then, if more frequent and regimented glucose monitoring is performed over time period 106 (e.g., about 72 times to about 96 times or more per day over about 14 days or more) followed by measurement of HbA1c 12102b and RPI 110b, the four sets of data in combination may be used to calculate one or more physiological parameters (k_{g iy}, k_{age}, and/or K) at time point 101.

Alternatively, in some embodiments, one or more measured RPI values 110a, 110b, two measured HbA1c values (a first measured HbA1c value 12102a at the beginning of a time period 106 and a second measured HbA1c value 12102b at the end of the time period 106), and a plurality of glucose levels 12104a measured during the time period 106 may be used to calculate one or more physiological parameters (k_{gly}, k_{age}, and/or K) at time point 101. In these embodiments, Equation 11 may be used to calculate k_{age}, and Equation 8 may be used to calculate k_{gly} and/or K at time point 101. In such embodiments, the plurality of glucose levels 12104a may be measured for about 10 days to about 30 days or longer with measurements being, on average, about 4 times daily (e.g., about every 6 hours) to about 24 times daily (e.g., about every 1 hour) or more often.

In the foregoing embodiments, the RPI value(s) can be measured at a time other than as illustrated because measured RPI values are relatively stable over time. Therefore, the RPI value(s) can be measured at any time during time period 106 and be applicable to these embodiments.

The foregoing embodiments are not limited to the example glucose level measurement time period and frequency ranges provided. Glucose levels may be measured over a time period of about a few days to about 300 days or more (e.g., about one week or more, about 10 days or more, about 14 days or more, about 30 days or more, about 60 days or more, about 90 days or more, about 120 days or more, and so on). In some embodiments, the time period is 7 days or more, preferably one to ten months, and less than one year. The frequency of such glucose levels may be, on average, about 14,400 times daily (e.g., a time interval t_{g} of about every 6 seconds) (or more often) to about 3 times daily (e.g., a time interval t_{g} of about every 8 hours) (e.g., 1,440 times daily (e.g., a time interval t_{g} of about every minute), about 288 times daily (e.g., a time interval t_{g} of about every 5 minutes), about 144 times daily (e.g., a time interval t_{g} of about every 10 minutes), about 96 times daily (e.g., a time interval t_{g} of about every 15 minutes), about 72 times daily (e.g., a time interval t_{g} of about every 20 minutes), about 48 times daily (e.g., a time interval t_{g} of about every 30 minutes), about 24 times daily (e.g., a time interval t_{g} of about every 1 hour), about 12 times daily (e.g., a time interval t_{g} of about every 2 hours), about 8 times daily (e.g., a time interval t_{g} of about every 3 hours), about 6 times daily (e.g., a time interval t_{g} of about every 4 hours), about 4 times daily (e.g., a time interval t_{g} of about every 6 hours), and so on). In some instances, less frequent monitoring (like once or twice daily) may be used where the glucose measurements occur at about the same time (within about 30 minutes) daily to have a more direct comparison of day-to-day glucose levels and reduce error in subsequent analyses.

The foregoing embodiments may further include calculating an error or uncertainty associated with the one or more physiological parameters. In some embodiments, the error may be used to determine if another HbA1c value (not illustrated) should be measured near time point 101, if one or more glucose levels 12104b should be measured (e.g., near time point 101), if the monitoring and analysis should be extended (e.g., to extend through time period 108 from time point 101 to time point 12103 including measurement of glucose levels 12104b during time period 108 and measurement of HbA1c value 12102c at time point 12103), and/or if the frequency of glucose level measurements 12104b in an extended time period 108 should be increased relative to the frequency of glucose level measurements 12104a during time period 106. In some embodiments, one or more of the foregoing actions may be taken when the error associated with k_{gly}, k_{age}, and/or K is at or greater than about 15%, preferably at or greater than about 10%, preferably at or greater than about 7%, and preferably at or greater than about 5%. When a subject has an existing disease condition (e.g., cardiovascular disease), a lower error may be preferred to have more stringent monitoring and less error in the analyses described herein.

Alternatively or when the error is acceptable, in some embodiments, one or more physiological parameters (k_{gly}, k_{age}, and/or K) at time point 101 may be used to determine one or more parameters or characteristics for a subject's personalized diabetes management (e.g., a cHbA1c at the end of time period 108, a personalized-target glucose range, and/or a treatment or change in treatment for the subject in the near future), each described in more detail further herein. In some instances, in addition to the foregoing embodiments, an HbA1c value may be measured at time point 12103 and the one or more physiological parameters recalculated and applied to a future time period (not illustrated).

Alternatively or additionally, two values for k_{age} can be estimated using Equation 8 and Equation 11. A comparison of these two values can be used to determine if another HbA1c value (not illustrated) should be measured near time point 101, if one or more glucose levels 12104b should be measured (e.g., near time point 101), if the monitoring and analysis should be extended (e.g., to extend through time period 108 from time point 101 to time point 12103 including measurement of glucose levels 12104b and measurement of HbA1c value 12102c at time point 12103), and/or if the frequency of glucose level measurements 12104b in an extended time period 108 should be increased relative to the frequency of glucose level measurements 12104a during time period 106. For example, if the two values of k_{age} are more than 10% different (e.g., the low value is not within 10% of the high value based on the high value), the individual's kₘₐt may be different than the typical kmat (3.47 day⁻¹). If a large difference is observed (e.g., more than 20% difference), the individual's kmat could be determined. If the individual's kmat is stable over a time period (e.g., three to six months), the determined individual's kmat should be used in place of the typical kmat in Equation 11 in the methods, systems, and devices described herein. Fluctuation in kmat could suggest other health problems.

The one or more physiological parameters and/or the one or more parameters or characteristics for a subject's personalized diabetes management can be measured and/or calculated for two or more times (e.g., time point 101 and time point 12103) and compared. For example, k_{gly} at time point 101 and time point 12103 may be compared. In another example, cHbA1c at time point 12103 and at a future time may be compared. Some embodiments, described further herein, may use such comparisons to (1) monitor progress and/or effectiveness of a subject's personalized diabetes management and, optionally, alter the subject's personalized diabetes management, (2) identify an abnormal or diseased physiological condition, and/or (3) identify subjects taking supplements and/or medicines that affect red blood cell production and/or affect metabolism.

Each of the example methods, devices, and systems described herein can utilize the one or more physiological parameters (k_{gly}, k_{age}, and K) and perform one or more related analyses (e.g., personalized-target glucose range, personalized-target average glucose, cHbA1c, and the like). The one or more physiological parameters (k_{gly}, k_{age}, and K) and related analyses may be updated periodically (e.g., about every 3 months to annually). The frequency of updates may depend on, among other things, the subject's glucose level and diabetes history (e.g., how well the subject stays within the prescribed thresholds), other medical conditions, and the like.

### Other Factors

In the embodiments described herein that apply the one or more physiological parameters (k_{gly}, k_{age}, and/or K), one or more other subject-specific parameters may be used in addition to the one or more physiological parameters. Examples of subject-specific parameters may include, but are not limited to, vital information (e.g., heart rate, body temperature, blood pressure, or any other vital information), body chemistry information (e.g., drug concentration, blood levels, troponin level, cholesterol level, or any other body chemistry information), meal data/information (e.g., carbohydrate amount, sugar amount, or any other information about a meal), activity information (e.g., the occurrence and/or duration of sleep and/or exercise), an existing medical condition (e.g., cardiovascular disease, heart valve replacement, cancer, and systemic disorder such as autoimmune disease, hormone disorders, and blood cell disorders), a family history of a medical condition, a current treatment, an age, a race, a gender, a geographic location (e.g., where a subject grew up or where a subject currently lives), a diabetes type, a duration of diabetes diagnosis, and the like, and any combination thereof.

### Systems

In some embodiments, determining the one or more physiological parameters (k_{gly}, k_{age}, and/or K) for a subject may be performed using a physiological parameter analysis system.

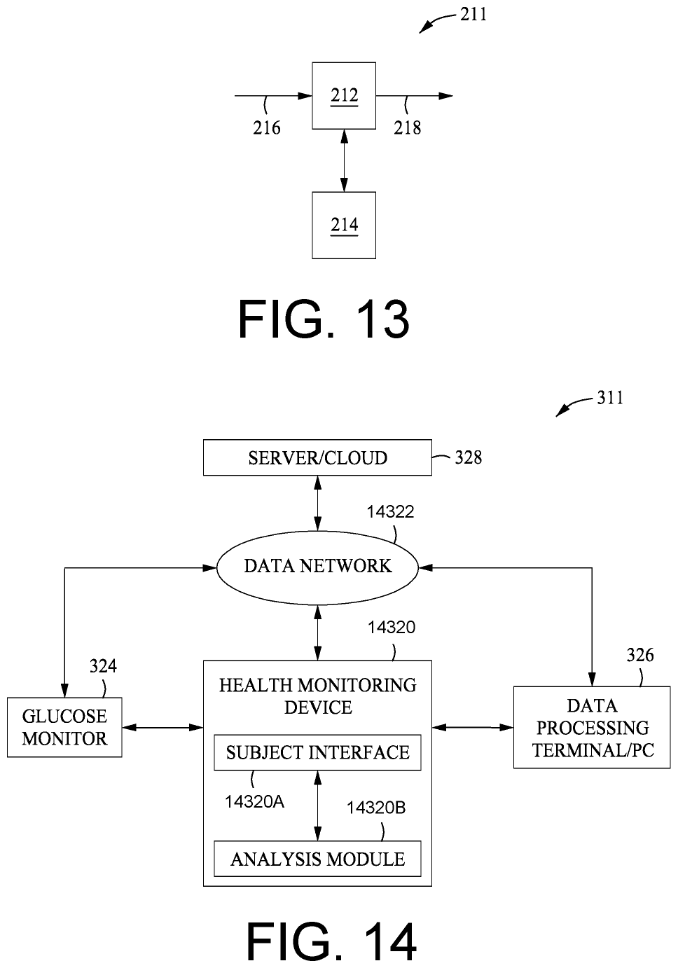

FIG. 13 illustrates an example of a physiological parameter analysis system 211 for providing physiological parameter analysis in accordance with some of the embodiments of the present disclosure. The physiological parameter analysis system 211 includes one or more processors 212 and one or more machine-readable storage media 214. The one or more machine-readable storage media 214 contains a set of instructions for performing a physiological parameter analysis routine, which are executed by the one or more processors 212.

In some embodiments, the instructions include receiving inputs 216 (e.g., one or more RPI values, one or more glucose levels, one or more HbA1c levels, one or more physiological parameters (k_{gly}, k_{age}, and/or K) previously determined, or more other subject-specific parameters, and/or one or more times associated with any of the foregoing), determining outputs 218 (e.g., one or more physiological parameters (k_{gly}, k_{age}, and/or K), an error associated with the one or more physiological parameters, one or more parameters or characteristics for a subject's personalized diabetes management (e.g., cHbA1c, a personalized-target glucose range, an average-target glucose level, a supplement or medication dosage, among other parameters or characteristics), a matched group of participants, and the like), and communicating the outputs 218. In some embodiments, communication of the inputs 216 may be via a user-interface (which may be part of a display), a data network, a server/cloud, another device, a computer, or any combination thereof, for example. In some embodiments, communication of the outputs 218 may be to a display (which may be part of a user-interface), a data network, a server/cloud, another device, a computer, or any combination thereof, for example.

A “machine-readable medium”, as the term is used herein, includes any mechanism that can store information in a form accessible by a machine (a machine may be, for example, a computer, network device, cellular phone, personal digital assistant (PDA), manufacturing tool, any device with one or more processors, and the like). For example, a machine-accessible medium includes recordable/non-recordable media (e.g., read-only memory (ROM), random access memory (RAM), magnetic disk storage media, optical storage media, flash memory devices, and the like).

In some instances, the one or more processors 212 and the one or more machine-readable storage media 214 may be in a single device (e.g., a computer, network device, cellular phone, PDA, an analyte monitor, and the like).

In some embodiments, a physiological parameter analysis system may include other components. FIG. 14 illustrates another example of a physiological parameter analysis system 311 for providing physiological parameter analysis in accordance with some of the embodiments of the present disclosure.

The physiological parameter analysis system 311 includes health monitoring device 14320 with subject interface 14320A and analysis module 14320B. The health monitoring device 14320 is, or may be, operatively coupled to data network 14322. Also provided in physiological parameter analysis system 311 is a glucose monitor 324 (e.g., in vivo and/or in vitro (ex vivo) devices or system) and a data processing terminal/personal computer (PC) 326, each operatively coupled to health monitoring device 14320 and/or data network 14322. Further shown in FIG. 14 is server/cloud 328 operatively coupled to data network 14322 for bi-directional data communication with one or more of health monitoring device 14320, data processing terminal/PC 326 and glucose monitor 324. Physiological parameter analysis system 311 within the scope of the present disclosure can exclude one or more of server/cloud 328, data processing terminal/PC 326 and/or data network 14322.

In certain embodiments, analysis module 14320B is programmed or configured to perform physiological parameter analysis and, optionally, other analyses (e.g., cHbA1c, personalized target glucose range, and others described herein). As illustrated, analysis module 14320B is a portion of the health monitoring device 14320 (e.g., executed by a processor therein). However, the analysis module 14320B may alternatively be associated with one or more of server/cloud 328, glucose monitor 324, and/or data processing terminal/PC 326. For example, one or more of server/cloud 328, glucose monitor 324, and/or data processing terminal/PC 326 may comprise a machine-readable storage medium (or media) with a set of instructions that cause one or more processors to execute the set of instructions corresponding to the analysis module 14320B.

While the health monitoring device 14320, the data processing terminal/PC 326, and the glucose monitor 324 are illustrated as each operatively coupled to the data network 14322 for communication to/from the server/cloud 328, one or more of the health monitoring device 14320, the data processing terminal/PC 326, and the glucose monitor 324 can be programmed or configured to directly communicate with the server/cloud 328, bypassing the data network 14322. The mode of communication between the health monitoring device 14320, the data processing terminal/PC 326, the glucose monitor 324, and the data network 14322 includes one or more wireless communication, wired communication, RF communication, BLUETOOTH® communication, WiFi data communication, radio frequency identification (RFID) enabled communication, ZIGBEE® communication, or any other suitable data communication protocol, and that optionally supports data encryption/decryption, data compression, data decompression and the like.

As described in further detail below, the physiological parameter analysis can be performed by one or more of the health monitoring device 14320, data processing terminal/PC 326, glucose monitor 324, and server/cloud 328, with the resulting analysis output shared in the physiological parameter analysis system 311.

Additionally, while the glucose monitor 324, the health monitoring device 14320, and the data processing terminal/PC 326 are illustrated as each operatively coupled to each other via communication links, they can be modules within one integrated device (e.g., sensor with a processor and communication interface for transmitting/receiving and processing data).

### Measuring Glucose and HbA1c Levels

The measurement of the plurality of glucose levels through the various time periods described herein may be done with in vivo and/or in vitro (ex vivo) methods, devices, or systems for measuring at least one analyte, such as glucose, in a bodily fluid such as in blood, interstitial fluid (ISF), subcutaneous fluid, dermal fluid, sweat, tears, saliva, or other biological fluid. In some instances, in vivo and in vitro methods, devices, or systems may be used in combination.

Examples of in vivo methods, devices, or systems measure glucose levels and optionally other analytes in blood or ISF where at least a portion of a sensor and/or sensor control device is, or can be, positioned in a subject's body (e.g., below a skin surface of a subject). Examples of devices include, but are not limited to, continuous analyte monitoring devices and flash analyte monitoring devices. Specific devices or systems are described further herein and can be found in U.S. Pat. No. 6,175,752 and U.S. Patent Application Publication No. 2011/0213225, the entire disclosures of each of which are incorporated herein by reference for all purposes. [0079] In vitro methods, devices, or systems (including those that are entirely non-invasive) include sensors that contact the bodily fluid outside the body for measuring glucose levels. For example, an in vitro system may use a meter device that has a port for receiving an analyte test strip carrying bodily fluid of the subject, which can be analyzed to determine the subject's glucose level in the bodily fluid. Additional devices and systems are described further below.

As described above the frequency and duration of measuring the glucose levels may vary from, on average, about 3 times daily (e.g., about every 8 hours) to about 14,400 times daily (e.g., about every 10 seconds) (or more often) and from about a few days to over about 300 days, respectively.

Once glucose levels are measured, the glucose levels may be used to determine the one or more physiological parameters (k_{gly}, k_{age}, and/or K) and, in some instances, other analyses (e.g., cHbA1c, personalized target glucose range, and others described herein). In some instances, such analyses may be performed with a physiological parameter analysis system. For example, referring back to FIG. 14, in some embodiments, the glucose monitor 324 may comprise a glucose sensor coupled to electronics for (1) processing signals from the glucose sensor and (2) communicating the processed glucose signals to one or more of health monitoring device 14320, server/cloud 328, and data processing terminal/PC 326.

The measurement of one or more HbA1c levels at the various times described herein may be according to any suitable method. Typically, HbA1c levels are measured in a laboratory using a blood sample from a subject. Examples of laboratory tests include, but are not limited to, a chromatography-based assay, an antibody-based immunoassay, and an enzyme-based immunoassay. HbA1c levels may also be measured using electrochemical biosensors.

The frequency of HbA1c level measurements may vary from, on average, monthly to annually (or less often if the average glucose level of the subject is stable).

Calculated HbA1c (cHbA1c)

Referring back to FIG. 14, in some embodiments, HbA1c levels may be measured with a laboratory test where the results are input to the server/cloud 328, the subject interface 14320A, and/or a display from the testing entity, a medical professional, the subject, or other user. Then, the HbA1c levels may be received by the one or more of health monitoring device 14320, server/cloud 328, and data processing terminal/PC 326 for analysis by one or more methods described herein.

After one or more physiological parameters (k_{gly}, k_{age}, and/or K) are calculated, a plurality of glucose measurements may be taken for a following time period and used for calculating HbA1c during and/or at the end of the following time period. For example, referring back to FIG. 12, one or more physiological parameters (k_{gly}, k_{age/}and/or K) may be calculated at time point 101 based on one or more measured RPI values 110a, 110b, measurements of the plurality of glucose levels 12104a over time period 106, a measured HbA1c level 12102b at the end of time period 106, and optionally a measured HbA1c level 12102a at the beginning of time period 106. Then, for a subsequent time period 108, a plurality of glucose levels 12104b may be measured. Then, during and/or at the end of the time period 108, Equation 8 can be used to determine a cHbA1c value (HbA1c_{z} of Equation 8) where HbA1co is the measured HbA1c level 12102b at the end of time period 106 (which is the beginning of time period 108), [G,] are the glucose levels or averaged glucose levels during time period 108 (or the portion of time period 108 where cHbA1c is determined during the time period 108), and the provided one or more physiological parameters (k_{gly}, k_{age}, and/or K) corresponding to time point 101 are used.

A subject's cHbA1c may be determined for several successive time periods based on the one or more physiological parameters (k_{gly}, k_{age}, and/or K) determined with the most recently measured HbA1c level, the most recently measured RPI value(s), and the intervening measurements of glucose levels. The RPI value may be measured periodically (e.g., every 6 months to a year) to recalculate k_{age}. The most recent RPI value or an average of two or more RPI values can be used in the calculation. The HbA1c may be measured periodically (e.g., every 6 months to a year) to recalculate the one or more physiological parameters. The time between remeasuring the RPI value and the measured HbA1c may depend on (1) the consistency of the measurements of glucose levels, (2) the frequency of the measurements of glucose levels, (3) a subject's and corresponding family's diabetic history, (4) the length of time the subject has been diagnosed with diabetes, (5) changes to a subject's personalized diabetes management (e.g., changes in medications/dosages, changes in diet, changes in exercise, and the like), (6) the presence of a disease or disorder that effects kₘₐₜ (e.g., anemia, a bone marrow disease, a genetic condition, an immune system disorder, and combinations thereof). For example, a subject with consistent measurements of glucose levels (e.g., a [G] with less than 5% variation) and frequent measurements of glucose levels (e.g., continuous glucose monitoring) may measure HbA1c levels less frequently than a subject who recently (e.g., within the last 6 months) changed the dosage of a glycation medication, even with consistent and frequent measurements of glucose levels.

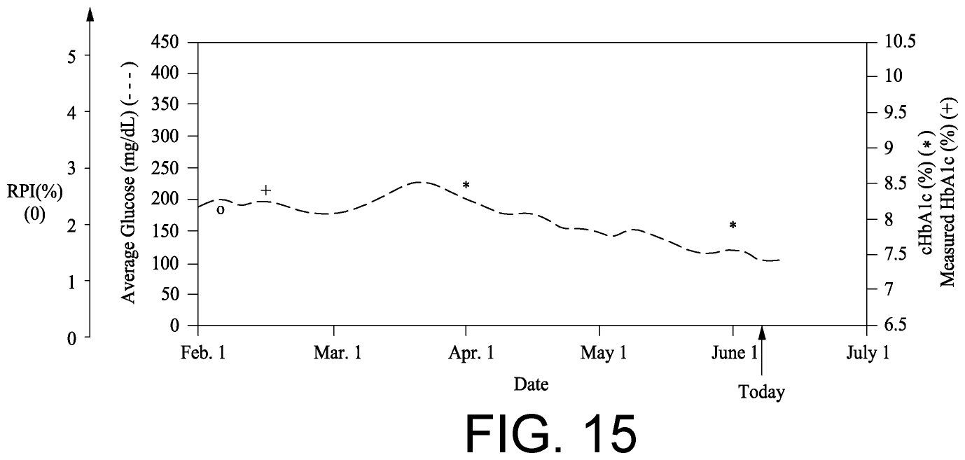

FIG. 15, with reference to FIG. 13, illustrates an example of a cHbA1c report that may be generated as an output 218 by a physiological parameter analysis system 211 of the present disclosure. The illustrated example report includes a plot of average glucose level over time. Also included on the report are the most recently measured RPI value (open circle), the most recently measured HbA1c level (cross), and cHbA1c levels (asterisks) calculated by the physiological parameter analysis system 211. While the most recently measured RPI value and the most recently measured HbA1c level are illustrated as being measured on different days, the two measurements can be done in the same visit to a health care provider.

Two cHbA1c levels are illustrated, but one or more cHbA1c levels may be displayed on the report, including a line that continuously tracks cHbA1c. Alternatively, the output 218 of the physiological parameter analysis system 211 may include a single number for a current or most recently calculated cHbA1c, a table corresponding to the data of FIG. 15, or any other report that provides a subject, healthcare provider, or the like with at least one cHbA1c level.

In some instances, the cHbA1c may be compared to a previous cHbA1c and/or a previous measured HbA1c level to monitor the efficacy of a subject's personalized diabetes management. For example, if a diet and/or exercise plan is being implemented as part of a subject's personalized diabetes management, with all other factors (e.g., medication and other diseases) equal, then changes in the cHbA1c compared to the previous cHbA1c and/or the previous measured HbA1c level may indicate if the diet and/or exercise plan is effective, ineffective, or a gradation therebetween.

In some instances, the cHbA1c may be compared to a previous cHbA1c and/or a previous measured HbA1c level to determine if another HbA1c measurement should be taken. For example, in the absence of significant glucose profile change, if the cHbA1c changes by 0.5 percentage units or more (e.g., changes from 7.0% to 6.5% or from 7.5% to 6.8%) as compared to the previous cHbA1c and/or the previous measured HbA1c level, another measured HbA1c level may be tested.

In some instances, a comparison of the cHbA1c to a previous cHbA1c and/or a previous measured HbA1c level may indicate if an abnormal or diseased physiological condition is present. For example, if a subject has maintained a cHbA1c and/or measured HbA1c level for an extended period of time, then if a change in cHbA1c is identified with no other obvious causes, the subject may have a new abnormal or diseased physiological condition. Indications of what that new abnormal or diseased physiological condition may be gleaned from the one or more physiological parameters (k_{gly}, k_{age}, and/or K). Details of abnormal or diseased physiological conditions relative to the one or more physiological parameters are discussed further herein.

### Personalized-Target Glucose Range

Typically, the glucose levels in subjects with diabetes are preferably maintained between 54 mg/dL and 180 mg/dl_. However, the kinetic model described herein (see Equation 6) illustrates that intracellular glucose levels are dependent on physiological parameters k_{gly}, k_{age}, and K. Therefore, a measured glucose level may not correspond to the actual physiological conditions in a subject. For example, a subject with a higher than normal K may glycate glucose more readily. Therefore, a 180 mg/dl_measured glucose level may be too high for the subject and, in the long run, potentially worsen the effects of the subject's diabetes. In another example, a subject with a lower than normal k_{gly} may glycate glucose to a lesser degree. Accordingly, at a 54 mg/dL glucose level, the subject's intracellular glucose level may be much lower making the subject feel weak and, in the long term, lead to the subject being hypoglycemic.

Using the accepted normal lower glucose limit (LGL) and the accepted normal HbA1c upper limit (AU), equations for a personalized lower glucose limit (GL) (Equation 13) and a personalized upper glucose limit (GU) (Equation 14) can be derived from Equation 6.

$$GL = ( LGL {*}^{3 / 4} J ) // {c}^{3 / 4} ? where {k}_{t}^{re} \hat{} is the {k}_{gly} for \\ a normal person and kjffi is the subject ’ s {k}_{gly} . \qquad Equation 13$$

$$GU = AU / ( K ( 1 - AU ) ) \qquad Equation 14$$

Equation 13 is based on k_{gly} because the lower limit of a glucose range is based on an equivalent intracellular glucose level. Equation 14 is based on K because the upper limit of a glucose range is based on an equivalent extracellular glucose level (e.g., the accepted normal HbA1c upper limit).

The currently accepted values for the foregoing are LGL=54 mg/dL, k_{g}^{e\hat{ }}=6.2*10⁻⁶ dL*mg⁻¹*day⁻¹, and AU=0.08 (i.e., 8%). Using the currently accepted values Equations 15 and 16 can be derived.

$$GL = 3.35 * {10}^{4} day {-}^{!} / k \qquad Equation 15$$

$$GU = 0.087 / K \qquad Equation 16$$

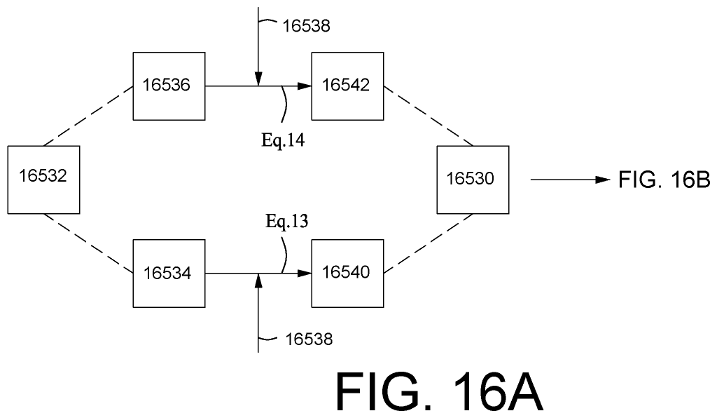

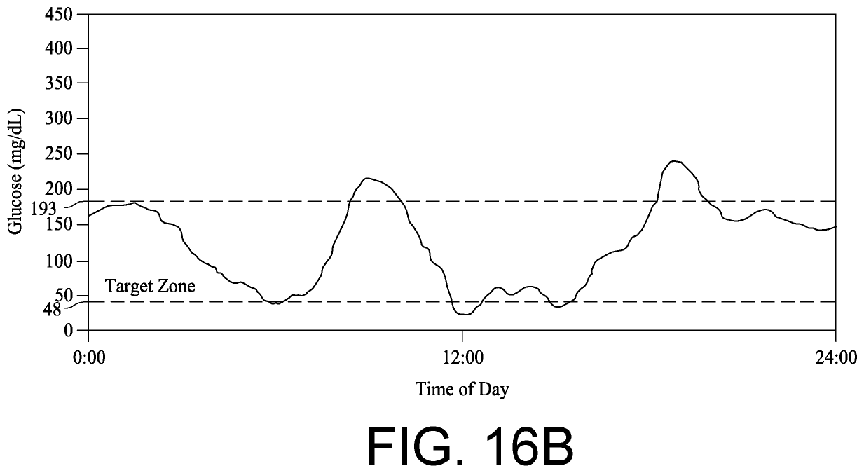

FIG. 16A illustrates an example of a method of determining a personalized-target glucose range 16530. A desired intracellular glucose range 16532 (e.g., the currently accepted glucose range) having a lower limit 16534 and an upper limit 16536 can be personalized using one or more determined physiological parameters (k_{gly}, k_{age}/and/or K) 16538 using Equation 13 and Equation 14, respectively. This results in a personalized lower glucose limit (GL) 16540 (Equation 13±7%) and a personalized upper glucose limit (GU) 16542 (Equation 14±7%) that define the personalized-target glucose range 16530. After one or more physiological parameters (k_{gly}, k_{age}, and/or K) are calculated, a personalized-target glucose range may be determined where the lower glucose limit may be altered according to Equation 13 (or Equation 15)±7% and/or the upper glucose limit may be altered according to Equation 14 (or Equation 16)±7%. The ±7% relative to each of the foregoing calculated values allows for a different value that is substantially close to the calculated value to be used, so that the personalized nature of the personalized-target glucose range 16530 is maintained. Alternatively, the ±7% can be ±10%, ±5%, or ±3%.

For example, a subject with a K of 4.5*10^{˜4} dL/mg and a k_{gly} of 7.0*10⁶ dL*mg⁻¹*day¹ may have a personalized-target glucose range of about 48±3.4 mg/dl_ to about 193±13.5 mg/dl_. Therefore, the subject may have a wider range of acceptable glucose levels than the currently practiced glucose range.

FIG. 16B, with reference to FIG. 13, illustrates an example of a personalized-target glucose range report that may be generated as an output 218 by a physiological parameter analysis system 211 of the present disclosure. The illustrated example report includes a plot of glucose level over a day relative to the foregoing personalized-target glucose range (area between the dashed lines). Alternatively, other reports may include, but are not limited to, an ambulatory glucose profile (AGP) plot, a numeric display of the personalized-target glucose range with the most recent glucose level measurement, and the like, and any combination thereof.

In another example, a subject with a K of 6.5*10⁻⁴ dL/mg and a k_{gly} of 6.0*10⁶ dL*mg⁻¹*day¹ may have a personalized-target glucose range of about 56±3.5 mg/dL to about 134±10 mg/dL. With the much-reduced upper glucose level limit, the subject's personalized diabetes management may include more frequent glucose level measurements and/or medications to stay substantially within the personalized-target glucose range.

In yet another example, a subject with a K of 5.0*10⁻⁴ dL/mg and a k_{gly} of 5.0*10⁻⁶ dL*mg⁻¹*day\hat{ } may have a personalized-target glucose range of about 67±4.5 mg/dL to about 174±12 mg/dL. This subject is more sensitive to lower glucose levels and may feel weak, hungry, dizzy, etc. more often if the currently practiced glucose range (54 mg/dL and 180 mg/dL) were used.

While the foregoing examples all include a personalized glucose lower limit and a personalized glucose upper limit, a personalized-target glucose range may alternatively include only the personalized glucose lower limit or the personalized glucose upper limit and use the currently practiced glucose lower or upper limit as the other value in the personalized-target glucose range.

The personalized-target glucose range may be determined and/or implemented in a physiological parameter analysis system. For example, a set of instructions or program associated with a glucose monitor and/or health monitoring device that determines a therapy (e.g., an insulin dosage) may use a personalized-target glucose range in such analysis. In some instances, a display or subject interface with display may display the personalized-target glucose range.

The personalized-target glucose range may be updated over time as one or more physiological parameters are recalculated.

### Personalized-Target Average Glucose

In some instances, a subject's personalized diabetes management may include having an HbA1c value target for a future time point. For example, referring to FIG. 12, a subject may have a measured RPI value 110b and a measured HbA1c value 12102b for time point 101 and a plurality of glucose level measurements prior thereto over time period 106. The subject's personalized diabetes management may include a target HbA1c value (AT) for time point 12103 that would correlate to improved health for the subject. Equation 17 can be used to calculate a personalized-target average glucose level (GT) for the next time period 108 and be based on the target HbA1c value (AT) and the subject's K calculated at time point 101.

$$GT = AT / ( K ( 1 - AT ) ) \qquad Equation 17$$

In some embodiments, a physiological parameter analysis system may determine an average glucose level for the subject during time period 108 and, in some instances, display the average glucose level and/or the target average glucose level. The subject may use the current average glucose level and the target average glucose level to self-monitor their progress over time period 108. In some instances, the current average glucose level may be transmitted (periodically or regularly) to a health care provider using a physiological parameter analysis system for monitoring and/or analysis.

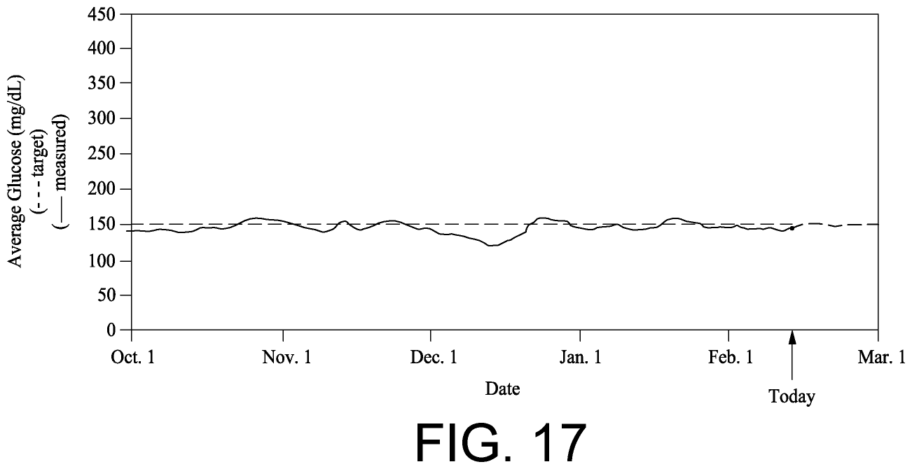

FIG. 17, with reference to FIG. 13, illustrates an example of a personalized-target average glucose report that may be generated as an output 218 by a physiological parameter analysis system 211 of the present disclosure. The illustrated example report includes a plot of a subject's average glucose (solid line) over time and the personalized-target average glucose (illustrated at 150 mg/dL, dashed line). Alternatively, other reports may include, but are not limited to, a numeric display of the personalized-target average glucose with the subject's average glucose level over a given time frame (e.g., the last 12 hours), and the like, and any combination thereof.

The personalized-target average glucose may be updated over time as one or more physiological parameters are recalculated.

### EXAMPLES

Data from 148 type 2 and 139 type 1 subjects enrolled in two previous clinical studies having six months of continuous glucose monitoring were analyzed. Only 90 subjects had sufficient data to meet the kinetic model assumptions described above having data with no continuous glucose data gap 12 hours or longer. Study participants had three HbA1c measurements, on days 1, 100 (±5 days), and 200 (±5 days), as well as frequent subcutaneous glucose monitoring throughout the analysis time period, which allowed for analysis of two independent data sections (days 1-100 and days 101-200) per participant.

The first data section (days 1-100) was used to numerically estimate individual k_{gly} and k_{age}, which allows prospective calculation of ending cHbA1c of the second data section (days 101-200). This ending cHbA1c can be compared with the observed ending HbA1c to validate the kinetic model described herein. For comparison, an estimated HbA1c for the second data section was calculated based on (1) 14-day mean and (2) 14-day weighted average glucose converted by the accepted regression model from the Ale-Derived Average Glucose (ADAG) study, which both assume k_{gly} is a constant, which as discussed previously is the currently accepted method of relating HbA1c to glucose measurements.

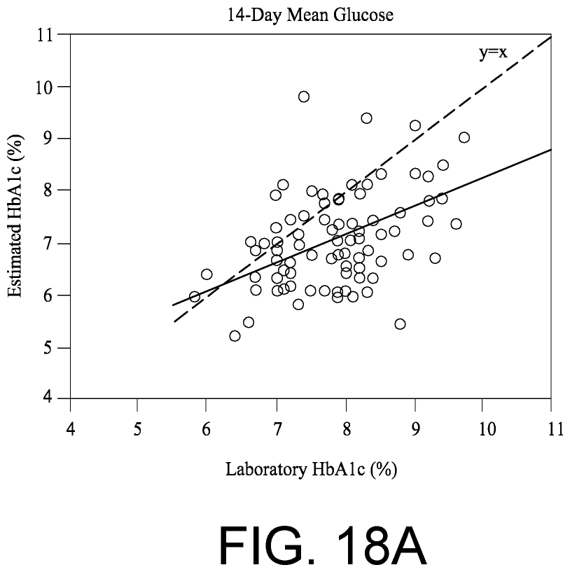

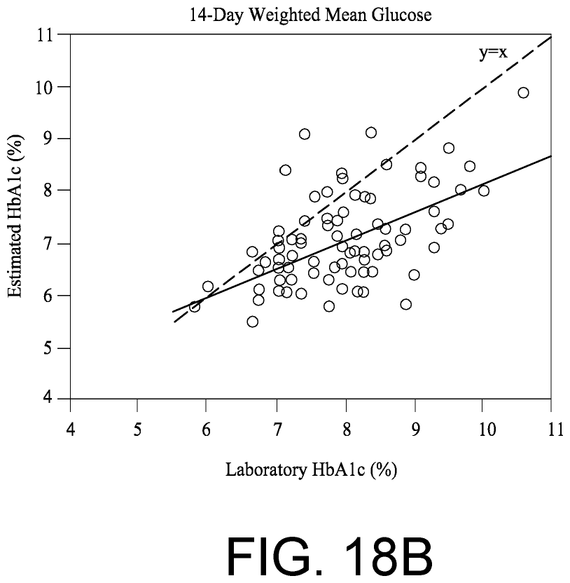

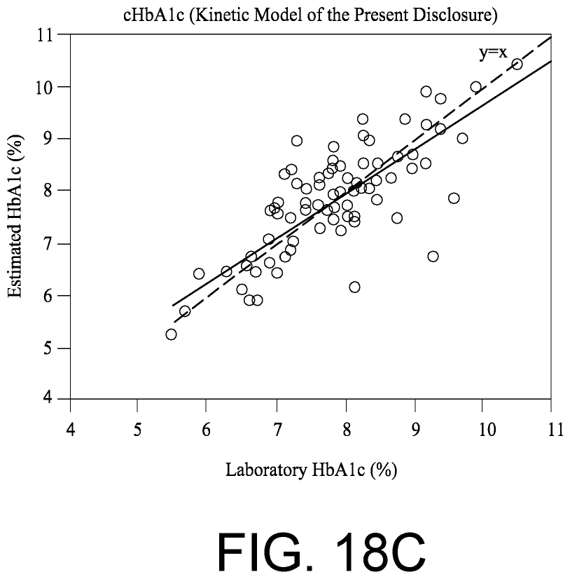

FIGS. 18A-C illustrate a comparison between the laboratory HbA1c levels at day 200 (±5 days) relative to the estimated HbA1c values, where the eHbA1c values in the 18A plot are calculated using the 14-day mean model, the eHbA1c values in the 18B plot are calculated using the 14-day weighted average model, and the cHbA1c values in the 18C plot are calculated using the kinetic model described herein (Equation 8). The solid line in all graphs illustrates the linear regression of the comparative HbA1c values for the corresponding models. The dashed line is a one-to-one line, where the closer the solid line linear regression is thereto, the better the model. Clearly, the kinetic model described herein models the data better, which illustrates that k_{age} and k_{gly} are individualized, which is a novel way to approach correlating HbA1c to glucose measurements.

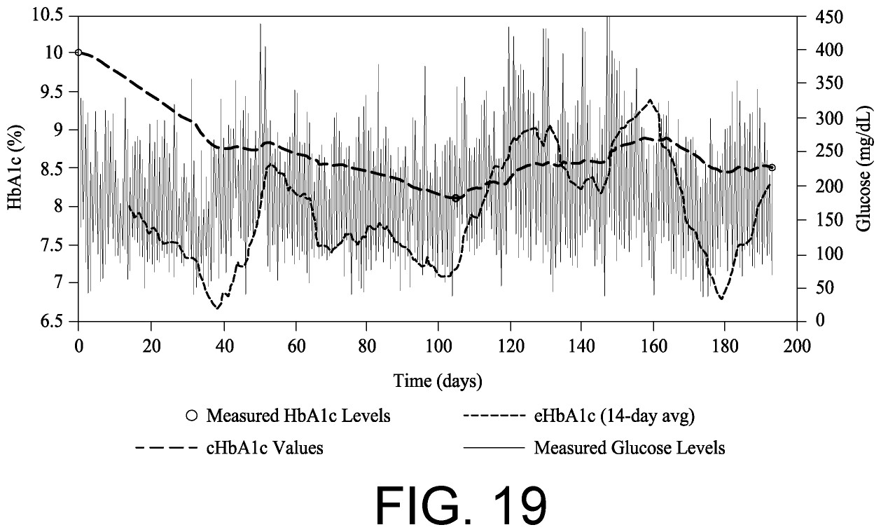

FIG. 19 illustrates an example study subject's data with the measured glucose levels (solid line), laboratory HbA1c readings (open circles), cHbA1c model values (long dashed line), and 14-day eHbA1c model values (dotted line). The cHbA1c model values in FIG. 19 were calculated using the physiological parameters (k_{age} and k_{gly}). The physiological parameters were calculated based on the first two laboratory HbA1c readings and glucose levels measured between the first two laboratory HbA1c readings. The 14-day eHbA1c values are glucose level 14-day running averages during the study.

The FIG. 19 example shows the dynamic nature of the glucose-to-cHbA1c and glucose-to-eHbA1c relationships. Additional examples were determined for type 1 and type 2 diabetes study participants across a range of prediction deviations: 25th, 50th and 75th percentiles for the cHbA1c method. In these examples, the disagreement between the cHbA1c from the 14-day average glucose indicates the exaggerated amplitude of variation inherent in the simple 14-day method.

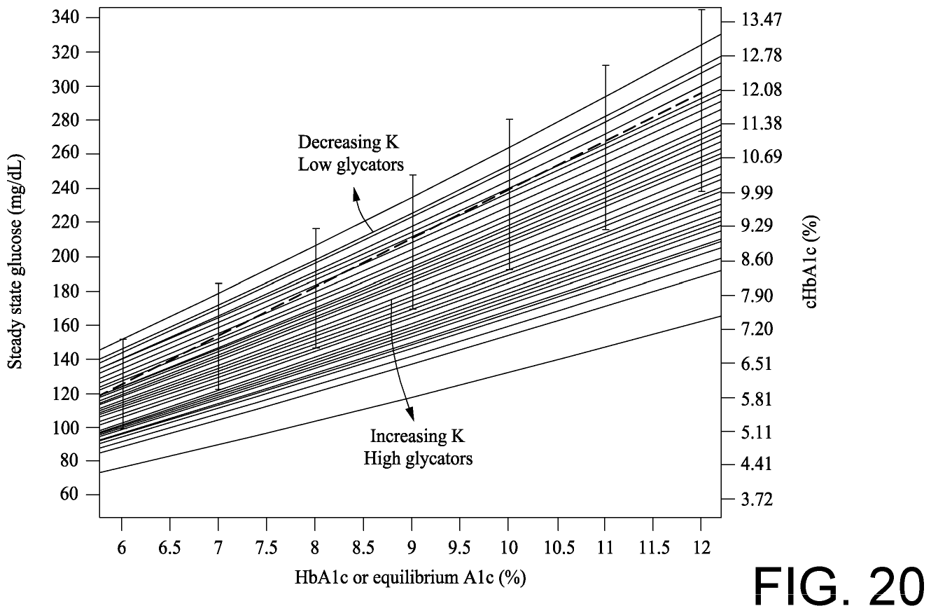

FIG. 20 illustrates the relationship between steady glucose and equilibrium HbA1c (1) as determined using the standard conversion of HbA1c to estimated average glucose (dashed line with error bars) and (2) as measured for the 90 participants (solid lines). These individual curves (solid lines) represent the agreement of average glucose with laboratory measure HbA1c under the condition of their average glucose level being stable for days-to-weeks. The model suggests that the relationship of glucose-to-HbA1c is not constant, with larger changes in glucose needed to achieve the same change in HbA1c as levels of the latter marker increase. Contrary to prior assessments of the glycation index, the kinetic model of the present disclosure suggests that an individual's glycation index will not be constant across all levels of HbA1c. Unlike eHbA1c, a key advantage of cHbA1c is its ability to account for individual variation in glycation. Individuals with lower K are “low glycators”, and have higher average glucose levels for a given HbA1c level, with the reverse being true for those with high K values.

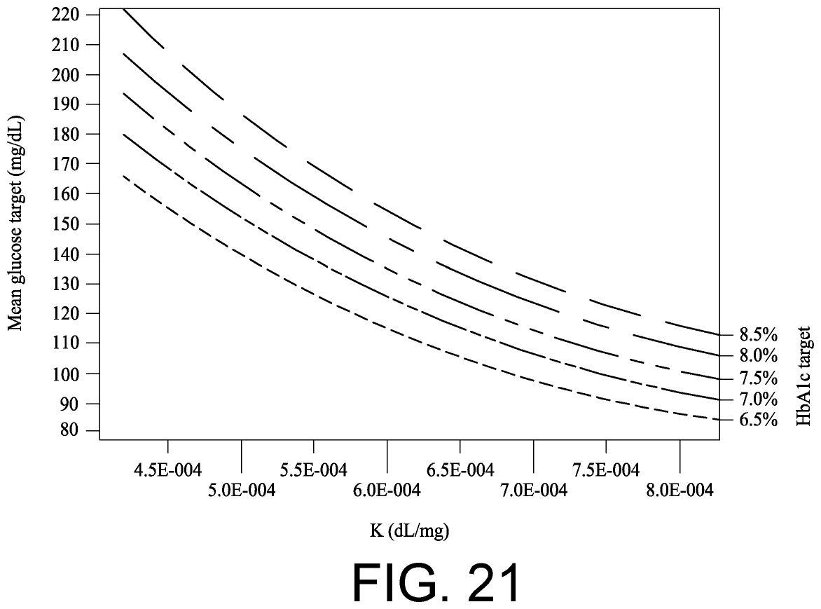

Using the kinetic model of the present disclosure, a relationship between K (dL/mg) and mean glucose level target (mg/dL) is illustrated in FIG. 21 plotted for varying HbA1c target values. That is, if a subject is targeting a specific HbA1c value (e.g., for a subsequent HbA1c measurement or cHbA1c estimation) and has a known K value (e.g., based on a plurality of measured glucose levels and at least one measured HbA1c), a mean glucose target can be derived and/or identified for the subject over the time period in which the subject is targeting the HbA1c value.

Additional details of methods, devices, and systems for determining physiological parameters related to the kinetics of red blood cell glycation, elimination, and generation within the body of a subject are set forth in U.S. Patent Publication No. 2018/0235524 to Dunn et al., International Publication No. WO2020/086934 to Xu, International Publication No. WO2021/108419 to Xu, International Publication No. WO2021/108431 to Xu, U.S. Provisional Patent Application No. 62/939,970, U.S. Provisional Patent Application No. 63/015,044, U.S. Provisional Patent Application No. 63/081,599, U.S. Provisional Patent Application No. 62/939,956, each of which is incorporated by reference in its entirety herein. Such physiological parameters can be used, for example, to calculate personalized glucose metrics or personalized analyte measurements: a more reliable calculated HbA1c (cHbA1c) or glucose-derived A1c (GD-A1c), adjusted HbA1c (aHbA1c or personalized A1c), adjusted cA1c (or cHbA1c adjusted by K_{age}), and/or a personalized target glucose range, among other things, for subject-personalized diagnoses, treatments, and/or monitoring protocols.

For purpose of illustration, not limitation, the processor in the reader device is configured to run the models described herein to calculate the physiological parameters and personalized glucose metrics. As embodied herein, the laboratory A1c measurement required to calculate the physiological parameters and the personalized glucose metrics can be received by the reader device, for example, not limitation, by using a camera (for example, not limitation, such as one built into the reader device) to scan a QR code which includes the relevant laboratory A1c data. As embodied herein, the laboratory A1c measurement can be received or retrieved by the reader device from a cloud-based database. As embodied herein, a home testing kit can be used to measure HbA1c in a blood sample and can be entered into the reader device by the user, instead of a laboratory A1c measurement.

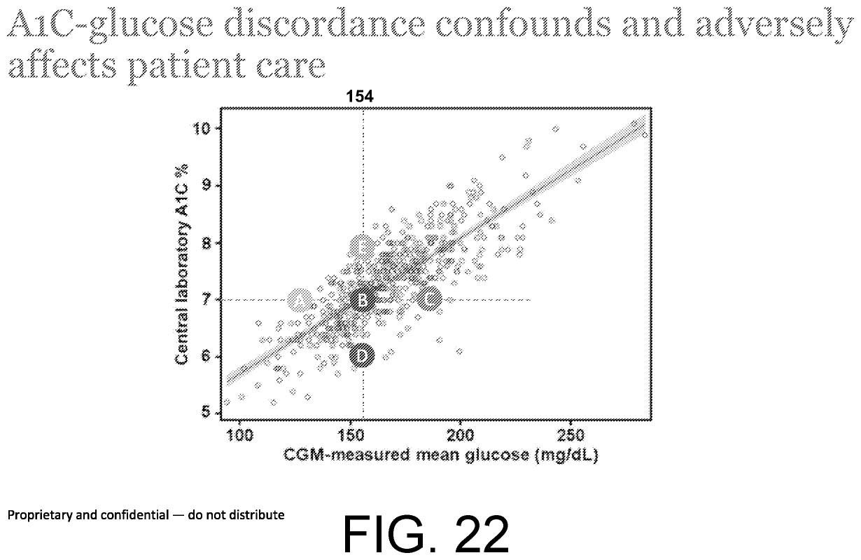

A1c-glucose discordance confounds and adversely affects subject care. For example, as shown in Table 1 below, subjects A, B, and C have the same laboratory measured A1c levels but different mean glucose levels (125 mg/dL, 154 mg/dL, and 188 mg/dL, respectively). Similarly, subjects B, D, and E have same mean glucose level of 154 mg/dL, but different laboratory measured A1c (7.0%, 6.0%, and 8.0%, respectively). This information is represented graphically in FIG. 22.

TABLE 1

| Subject | Mean Glucose (mg/dL) | Lab A1c % |
|---|---|---|
| A | 125 | 7.0 |
| B | 154 | 7.0 |
| C | 188 | 7.0 |
| D | 154 | 6.0 |
| E | 154 | 8.0 |

Models described herein allow quantitative removal of red blood cell artifacts, thereby improving hyperglycemia risk assessment. For example, for illustration not limitation, consider the subjects A-E with the following characteristics:

TABLE 2

| | RBC Lifespan | | Personalized A1c |
|---|---|---|---|
| Subject | (days) | Lab A1c (%) | (%) |
| A | 123 | 7.0 | 6.0 |
| B | 87 | 7.0 | 8.4 |
| C | 110 | 7.0 | 6.7 |
| D | 89 | 6.0 | 7.1 |
| E | 121 | 8.0 | 6.9 |

As can be seen in Table 2, subjects A, B, and C have different RBC lifespan (or as measured in days (123, 87, and 110, respectively) but the same laboratory measured Ale of 7.0%. Based on the different RBC lifespan, subject A, B, and C's personalized A1c or adjusted A1c, as measured by the models disclosed above, is 6, 8.4, and 6.7, respectively. Since the laboratory measured A1c for the three subjects is the same, their respective medical providers may view all three as diabetic and prescribe the same treatment regimen based on these values. However, because of their differing RBC lifespan, their glycemic control is in fact very different, as demonstrated by their starkly different personalized A1c. Indeed, based on their respective personalized A1c, subject A is pre-diabetic (based on personalized A1c of 6.0), subject B is clearly diabetic (based on personalized A1c of 8.4), and subject C is also diabetic (based on personalized A1c of 6.7). Accordingly, subjects A, B, and C in fact may need different treatment regimens. Similarly, although subject D may be viewed as pre-diabetic based on laboratory A1c of 6.0, they would be considered diabetic based on personalized A1c of 7.1. Further, subject E would be considered diabetic based on a laboratory A1c of 8.0, but would be considered pre-diabetic based on personalized A1c of 6.9.

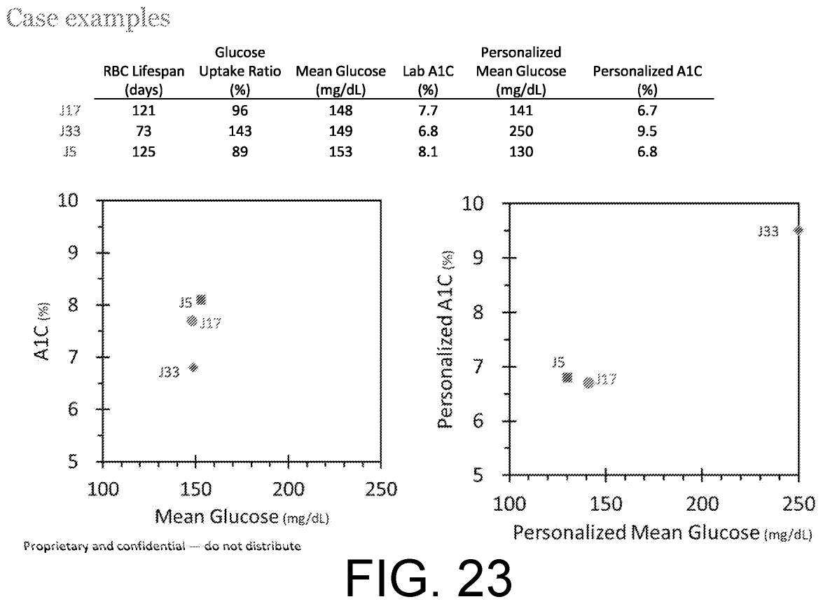

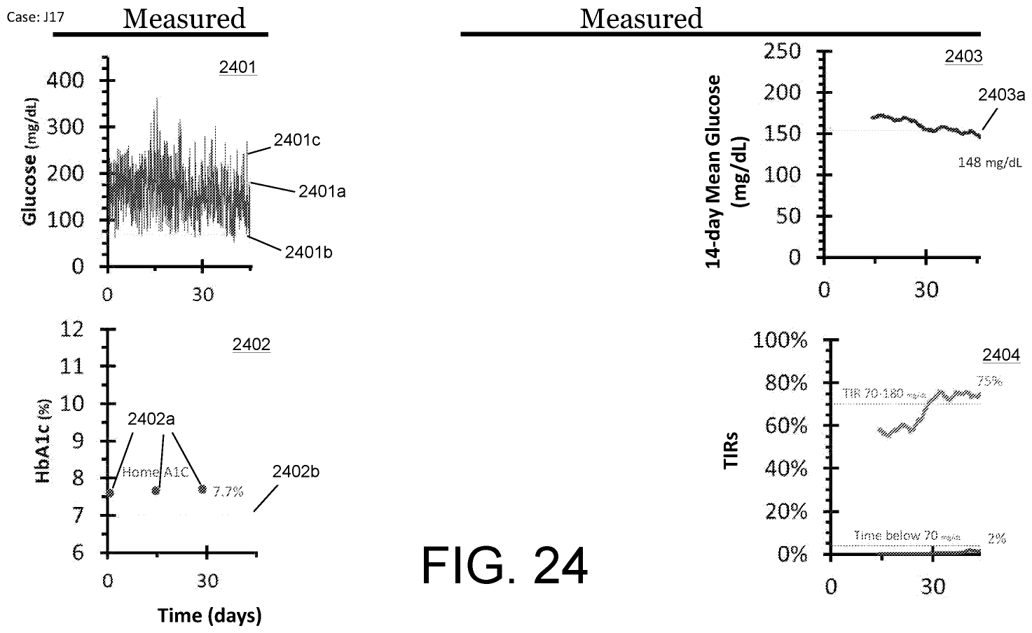

FIGS. 23-29 provide exemplary case studies illustrating the application of the models as described herein. For example, as can be seen in FIG. 23, exemplary subjects J17, J33, and J5 have a measured mean glucose of 148 mg/dL, 149 mg/dL, and 153 mg/dL, respectively, and laboratory A1c of 7.7%, 6.8%, and 8.1%. However, their personalized mean glucose and personalized A1c, as determined using the models described herein, differ significantly. Specifically, J17, J33, and J5 have a personalized mean glucose of 141 mg/dL, 250 mg/dL, and 130 mg/dL, respectively, and laboratory A1c of 6.7%, 9.5%, and 6.8%. Notably, J33's lab measured glucose metrics are starkly different than their personalized glucose metrics. FIG. 23 provides a graphical representation of these metrics. These and other metrics shown in FIG. 23 can also be seen in FIGS. 24-29.

### Example Embodiments of Sensor Results Interfaces

FIGS. 2D to 2I depict example embodiments of sensor results interfaces or GUIs for analyte monitoring systems. In accordance with the disclosed subject matter, the sensor results GUIs described herein are configured to display analyte data and other health information through a user interface application (e.g., software) installed on a reader device, such as a smart phone or a receiver, like those described with respect to FIG. 2B. Those of skill in the art will also appreciate that a user interface application with a sensor results interface or GUI can also be implemented on a local computer system or other computing device (e.g., wearable computing devices, smart watches, tablet computer, etc.).

Referring first to FIG. 2D, sensor results GUI 235 depicts an interface comprising a first portion 236 that can include a numeric representation of a current analyte concentration value (e.g., a current glucose value), a directional arrow to indicate an analyte trend direction, and a text description to provide contextual information such as, for example, whether the user's analyte level is in range (e.g., “Glucose in Range”). According to embodiments, first portion 236 can include a numeric representation of a personalized analyte concentration value (e.g., a personalized glucose value), as determined using a kinetic model as disclosed herein below. First portion 236 can also comprise a color or shade that is indicative of an analyte concentration or trend. For example, as shown in FIG. 2D, first portion 236 is a green shade, indicating that the user's analyte level (for example, not limitation, current or personalized glucose level) is within a target range. According to some embodiments, for example, a red shade can indicate an analyte level below a low analyte level threshold, an orange shade can indicate an analyte level above a high analyte level threshold, and an yellow shade can indicate an analyte level outside a target range. According to embodiments, the target range can be a personalized target glucose range as determined using a kinetic model as disclosed herein below.

In addition, according to some embodiments, sensor results GUI 235 also includes a second portion 237 comprising a graphical representation of analyte data. In particular, second portion 237 includes an analyte trend graph reflecting an analyte concentration, as shown by the y-axis, over a predetermined time period, as shown by the x-axis. According to embodiments, second portion 237 can include a personalized analyte trend graph reflecting a personalized analyte concentration, as determined using a kinetic model as disclosed herein below, as shown by the y-axis, over a predetermined time period, as shown by the x-axis. In some embodiments, the predetermined time period can be shown in five-minute increments, with a total of twelve hours of data. Those of skill in the art will appreciate, however, that other time increments and durations of analyte data can be utilized and are fully within the scope of this disclosure. Second portion 237 can also include a point 239 on the analyte trend graph to indicate the current analyte concentration value, a shaded green area 240 to indicate a target analyte range, and two dotted lines 238a and 238b to indicate, respectively, a high analyte threshold and a low analyte threshold. According to embodiments, point 239 on a personalized analyte trend graph can indicate the current personalized concentration value, shaded green area 240 to indicate a personalized target analyte range, and/or two dotted lines 238a and 238b to indicate, respectively, a personalized high analyte threshold and a personalized low analyte threshold. According to some embodiments, GUI 235 can also include a third portion 241 comprising a graphical indicator and textual information representative of a remaining amount of sensor life.

Referring next to FIG. 2E, another example embodiment of a sensor results GUI 245 is depicted. In accordance with the disclosed subject matter, first portion 236 is shown in a yellow shade to indicate that the user's current analyte concentration is not within a target range. According to embodiments, the currently analyte concentration can include a current personalized analyte concentration, and/or target range can be a personalized target range, as determined using a kinetic model as described herein. In addition, second portion 237 includes: an analyte trend line 241 which can reflect historical analyte levels over time and a current analyte data point 239 to indicate the current analyte concentration value (shown in yellow to indicate that the current value is outside the target range). According to embodiments, analyte trend line 241 can include historical personalized analyte levels over a time current analyte data point 239 can indicate personalized analyte concentration value.

According to another aspect of the embodiments, data on sensor results GUI 245 is automatically updated or refreshed according to an update interval (e.g., every second, every minute, every 5 minutes, etc.). For example, according to many of the embodiments, as analyte data is received by the reader device, sensor results GUI 245 will update: (1) the current analyte concentration value shown in first portion 236, and (2) the analyte trend line 241 and current analyte data point 239 show in second portion 237. Furthermore, in some embodiments, the automatically updating analyte data can cause older historical analyte data (e.g., in the left portion of analyte trend line 241) to no longer be displayed. According to embodiments, current analyte concentration value can include current personalized current value, analyte trend line 241 can include personalized analyte trend line 241, and current analyte data point 239 can include a current personalized analyte data point 239.

FIG. 2F is another example embodiment of a sensor results GUI 250. According to the depicted embodiment, sensor results GUI 250 includes first portion 236 which is shown in an orange shade to indicate that the user's analyte levels are above a high glucose threshold (e.g., greater than 250 mg/dL). According to embodiments, the user's analyte levels shown can include a current personalized analyte concentration, and high glucose threshold can include a personalized high glucose threshold. Sensor results GUI 250 also depicts health information icons 251, such as an exercise icon or an apple icon, to reflect user logged entries indicating the times when the user had exercised or eaten a meal.

FIG. 2G is another example embodiment of a sensor results GUI 255. According to the depicted embodiments, sensor results GUI 255 includes first portion 236 which is also shown in an orange shade to indicate that the user's analyte levels are above a high glucose threshold. As discussed above, according to embodiments, user's analyte levels shown can include a current personalized analyte concentration, and high glucose threshold can include a personalized high glucose threshold. As can be seen in FIG. 2G, first portion 236 does not report a numeric value but instead displays the text “HI” to indicate that the current analyte concentration value is outside a glucose reporting range high limit. Although not depicted in FIG. 2G, those of skill in the art will understand that, conversely, an analyte concentration below a glucose reporting range low limit will cause first portion 236 not to display a numeric value, but instead, the text “LO”. According to embodiments, first portion 236 can display the text “HI” to indicate that the personalized analyte concentration value is outside a personalized glucose reporting range high limit, and, conversely, first portion 236 would display “LO” when a personalized analyte concentration is below a glucose reporting range low limit.

FIG. 2H is another example embodiment of a sensor results GUI 260. According to the depicted embodiments, sensor results GUI 260 includes first portion 236 which is shown in a green shade to indicate that the user's current analyte level is within the target range. According to embodiments, user's current analyte levels can include a current personalized analyte level, and the target range can include a personalized target range. In addition, according to the depicted embodiments, first portion 236 of GUI 260 includes the text, “GLUCOSE GOING LOW,” which can indicate to the user that his or her analyte concentration value is predicted to drop below a predicted low analyte level threshold within a predetermined amount of time (e.g., predicted glucose will fall below 75 mg/dL within 15 minutes). Those of skill in the art will understand that if a user's analyte level is predicted to rise above a predicted high analyte level threshold within a predetermined amount of time, sensor results GUI 260 can display a “GLUCOSE GOING HIGH” message. According to embodiments, analyte concentration value can include a personalized analyte concentration value, and predicted low analyte level and predicted high analyte level can include a predicted personalized low analyte level and a predicted high analyte level, respectively.

FIG. 2I is another example embodiment of a sensor results GUI 265. According to the depicted embodiments, sensor results GUI 265 depicts first portion 236 when there is a sensor error. In accordance with the disclosed subject matter, first portion 236 includes three dashed lines 266 in place of the current analyte concentration value to indicate that a current analyte value is not available. According to embodiments, current analyte concentration value can include a current personalized analyte concentration value. In some embodiments, three dashed lines 266 can indicate one or more error conditions such as, for example, (1) a no signal condition; (2) a signal loss condition; (3) sensor too hot/cold condition; or (4) a glucose level unavailable condition. Furthermore, as can be seen in FIG. 2I, first portion 236 comprises a gray shading (instead of green, yellow, orange, or red) to indicate that no current analyte data (or current personalized analyte data) is available. In addition, according to another aspect of the embodiments, second portion 237 can be configured to display the historical analyte data in the analyte trend graph, even though there is an error condition preventing the display of a numeric value for a current analyte concentration in first portion 236. According to embodiments, historical analyte data can include historical personalized analyte data. However, as shown in FIG. 2I, no current analyte concentration value data point is shown on the analyte trend graph of second portion 237.

FIG. 2J is a glucose monitoring data interface which includes a graphical representation of the glucose monitoring data (right y-axis) for 200 days, superimposed with three laboratory HbA1c values (left y-axis) and the estimated HbA1c values (left y-axis) based on the 14-day eHbA1c model as disclosed in International Publication No. WO2021/108419 to Xu and WO2020/086934 to Xu, which are incorporated by reference in its entirety herein. As illustrated, the estimated HbA1c derived from the 14-day HbA1c model has very dramatic changes over time. However, it is unlikely that HbA1c can change this fast.

FIG. 2K is a glucose monitoring data interface which includes the graphical representation of FIG. 2J superimposed with a calculated HbA1c (left y-axis) for the first 100 days determined using k_{gly} and k_{age} per the methods described in International Publication No. WO2021/108419 and WO2020/086934 to Xu, which are incorporated by reference in its entirety herein.

FIG. 2L is a glucose monitoring data interface which includes the graphical representation of FIG. 2K superimposed with the calculated HbA1c (extension from day 100 to day 200, left y-axis) for the following 100 days using the k_{gly} and k_{age} determined relative to FIG. 2K per the methods described in International Publication No. WO2021/108419 and WO2020/086934 to Xu, which are incorporated by reference in its entirety herein. The third HbA1c value was not considered in this method, but the model described, predicted the measured value of the third HbA1c value, which illustrates that the model described herein is in close agreement with reality.

### Example Embodiments of Time-In-Ranges Interfaces

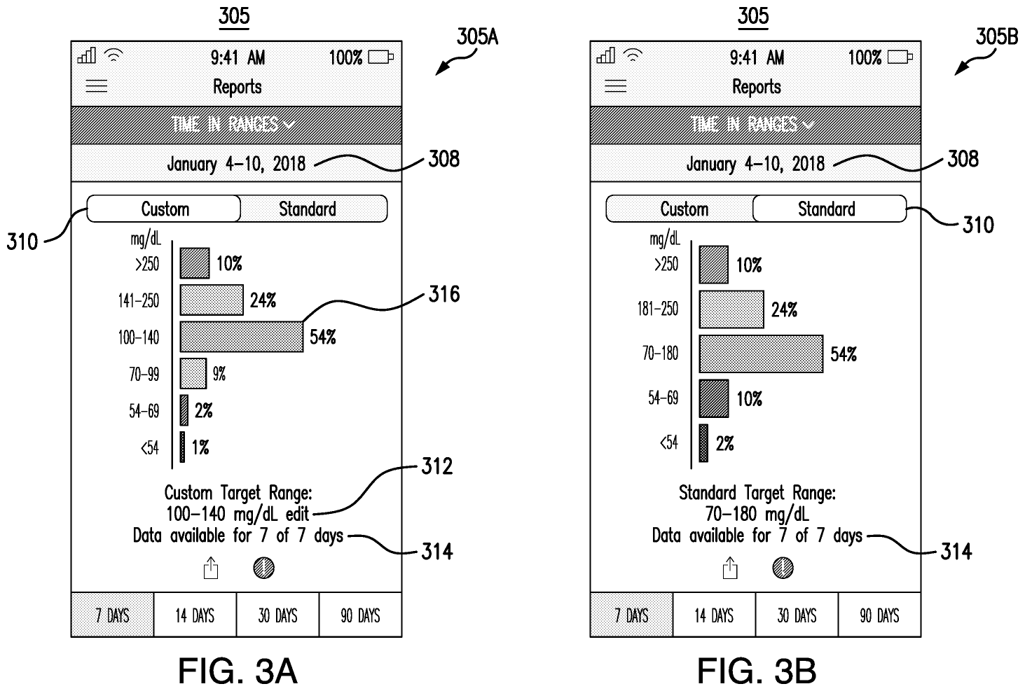

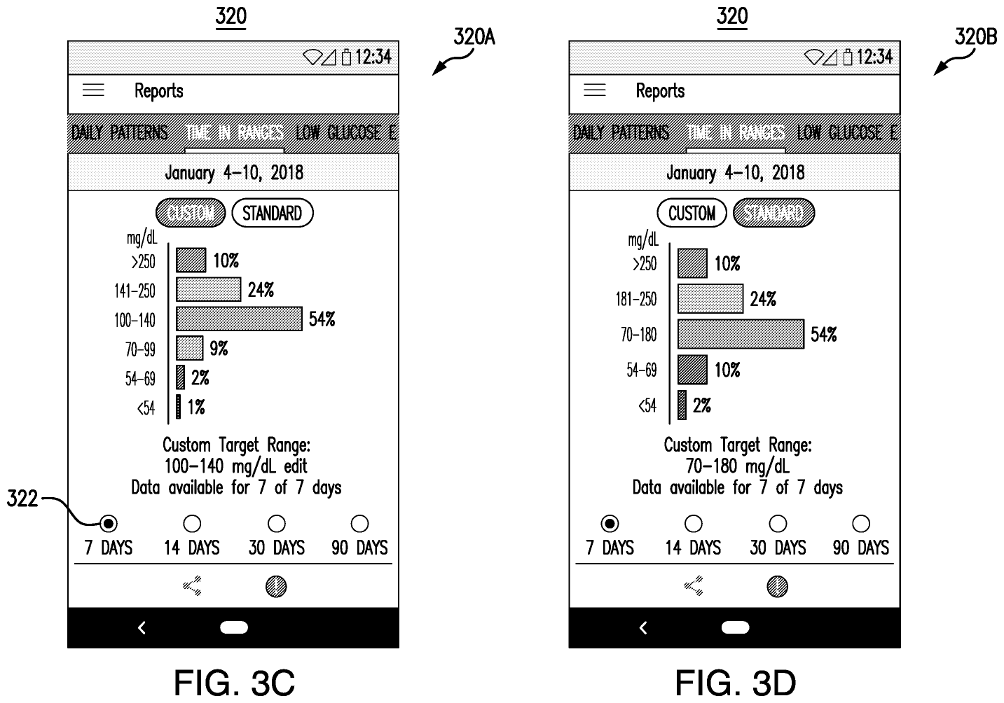

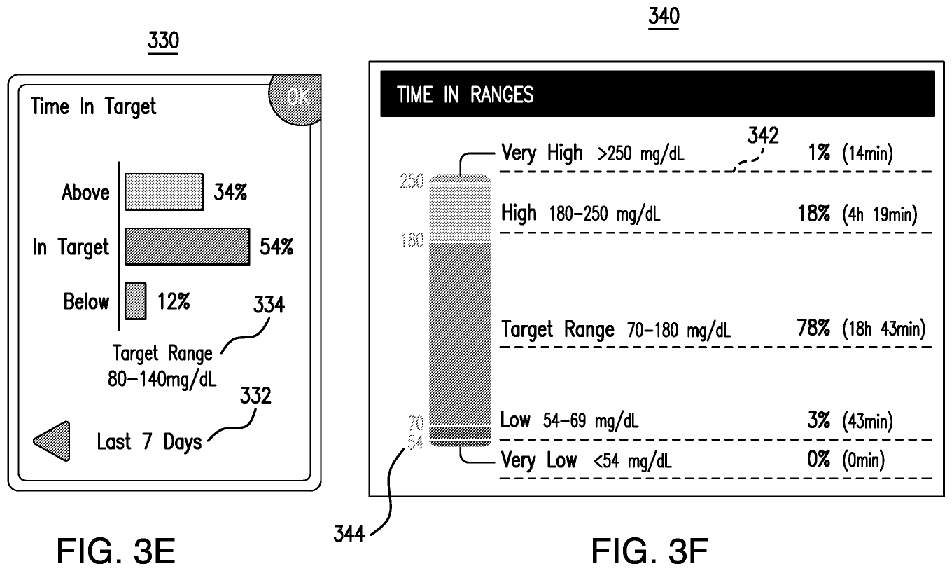

FIGS. 3A to 3F depict example embodiments of GUIs for analyte monitoring systems. In particular, FIGS. 3A to 3F depict Time-in-Ranges (also referred to as Time-in-Range and/or Time-in-Target) GUIs, each of which comprise a plurality of bars or bar portions, wherein each bar or bar portion indicates an amount of time that a user's analyte level is within a predefined analyte range correlating with the bar or bar portion. In some embodiments, for example, the amount of time can be expressed as a percentage of a predefined amount of time. According to embodiments, FIGS. 3A to 3F, as described below, can also depict personalized Time-in-Ranges (also referred to as personalized Time-in-Target) GUIs, each of which comprise a plurality of bars or bar portions, wherein each bar or bar portion indicates an amount of time that a user's personalized analyte level is within a predefined personalized analyte range correlating with the bar or bar portion.

Turning to FIGS. 3A and 3B, an example embodiment of a Time-in-Ranges GUI 305 is shown, wherein Time-in-Ranges GUI 305 comprises a “Custom” Time-in-Ranges view 305A and a “Standard” Time-in-Ranges view 305B, with a slidable element 310 that allows the user to select between the two views. In accordance with the disclosed subject matter, Time-in-Ranges views 305A, 305B can each comprise multiple bars, wherein each bar indicates an amount of time that a user's analyte level is within a predefined analyte range correlating with the bar. According to embodiments, user's analyte level can include personalized analyte level. In some embodiments, Time-in-Ranges views 305A, 305B further comprise a date range indicator 308, showing relevant dates associated with the displayed plurality of bars, and a data availability indicator 314, showing the period(s) of time in which analyte data is available for the displayed analyte data (e.g., “Data available for 7 of 7 days”).

Referring to FIG. 3A, “Custom” Time-in-Ranges view 305A includes six bars comprising (from top to bottom): a first bar indicating that the user's glucose range is above 250 mg/dL for 10% of a predefined amount of time, a second bar indicating that the user's glucose range is between 141 and 250 mg/dL for 24% of the predefined amount of time, a third bar 316 indicating that the user's glucose range is between 100 and 140 mg/dL for 54% of the predefined amount of time, a fourth bar indicating that the user's glucose range is between 70 and 99 mg/dL for 9% of the predefined amount of time, a fifth bar indicating that the user's glucose range is between 54 and 69 mg/dL for 2% of the predefined amount of time, and a sixth bar indicating that the user's glucose range is less than 54 mg/dL for 1% of the predefined amount of time. Those of skill in the art will recognize that the glucose ranges and percentages of time associated with each bar can vary depending on the ranges defined by the user and the available analyte data of the user, and that user's glucose range can include user's personalized glucose range. Furthermore, although FIGS. 3A and 3B show a predefined amount of time 314 equal to seven days, those of skill in the art will appreciate that other predefined amounts of time can be utilized (e.g., one day, three days, fourteen days, thirty days, ninety days, etc.), and are fully within the scope of this disclosure.

According to another aspect of the embodiments, “Custom” Time-in-Ranges view 305A also includes a user-definable custom target range 312 that includes an actionable “edit” link that allows a user to define and/or change the custom target range. As shown in “Custom” Time-in-Ranges view 305A, the custom target range 312 has been defined as a glucose range between 100 and 140 mg/dL and corresponds with third bar 316 of the plurality of bars. Those of skill in the art will also appreciate that, in other embodiments, more than one range can be adjustable by the user, and such embodiments are fully within the scope of this disclosure. According to embodiments, custom target range 312 can include custom personalized target ranges.

Referring to FIG. 3B, “Standard” Time-in-Ranges view 305B includes five bars comprising (from top to bottom): a first bar indicating that the user's glucose range is above 250 mg/dL for 10% of a predefined amount of time, a second bar indicating that the user's glucose range is between 181 and 250 mg/dL for 24% of the predefined amount of time, a third bar indicating that the user's glucose range is between 70 and 180 mg/dL for 54% of the predefined amount of time, a fourth bar indicating that the user's glucose range is between 54 and 69 mg/dL for 10% of the predefined amount of time, and a fifth bar indicating that the user's glucose range is less than 54 mg/dL for 2% of the predefined amount of time. As with the “Custom” Time-in-Ranges view 305A, those of skill in the art will recognize that the percentages of time associated with each bar can vary depending on the available analyte data of the user. Additionally, according to embodiments, the user's glucose range can include user's personalized glucose range, and the numerical glucose ranges associated with the five bars can be adjusted for a user's personalized glucose range. For example, not limitation, personalized glucose ranges can for each of the five bars can be calculated using the models as disclosed herein below. Unlike the “Custom” Time-in-Ranges view 305A, however, the glucose ranges shown in “Standard” view 305B cannot be adjusted by the user.

FIGS. 3C and 3D depict another example embodiment of Time-in-Ranges GUI 320 with multiple views, 320A and 320B, which are analogous to the views shown in FIGS. 3A and 3B, respectively. According to some embodiments, Time-in-Ranges GUI 320 can further include one or more selectable icons 322 (e.g., radio button, check box, slider, switch, etc.) that allow a user to select a predefined amount of time over which the user's analyte data will be shown in the Time-in-Range GUI 320. For example, as shown in FIGS. 3C and 3D, selectable icons 322 can be used to select a predefined amount of time of seven days, fourteen days, thirty days, or ninety days. Those of skill in the art will appreciate that other predefined amounts of time can be utilized and are fully within the scope of this disclosure.

FIG. 3E depicts an example embodiment of a Time-in-Target GUI 330, which can be visually output to a display of a reader device (e.g., a dedicated reader device, a meter device, etc.). In accordance with the disclosed subject matter, Time-in-Target GUI 330 includes three bars comprising (from top to bottom): a first bar indicating that the user's glucose range is above a predefined target range for 34% of a predefined amount of time, a second bar indicating that the user's glucose range is within the predefined target range for 54% of the predefined amount of time, and a third bar indicating that the user's glucose range is below the predefined target range for 12% of the predefined amount of time. Those of skill in the art will recognize that the percentages of time associated with each bar can vary depending on the available analyte data of the user, the user's glucose range can include user's personalized glucose range. Furthermore, although FIG. 3E shows a predefined amount of time 332 equal to the last seven days and a predefined target range 334 of 80 to 140 mg/dL, those of skill in the art will appreciate that other predefined amounts of time (e.g., one day, three days, fourteen days, thirty days, ninety days, etc.) and/or predefined target ranges (e.g., 70 to 180 mg/dL) can be utilized, and are fully within the scope of this disclosure. According to embodiments, predefined target range can be a predefined personalized target range determined using a kinetic model as disclosed herein.

FIG. 3F depicts another example embodiment of a Time-in-Ranges GUI 340, which includes a single bar comprising five bar portions including (from top to bottom): a first bar portion indicating that the user's glucose range is “Very High” or above 250 mg/dL for 1% (14 minutes) of a predefined amount of time, a second bar portion indicating that the user's glucose range is “High” or between 180 and 250 mg/dL for 18% (4 hours and 19 minutes) of the predefined amount of time, a third bar portion indicating that the user's glucose range is within a “Target Range” or between 70 and 180 mg/dL for 78% (18 hours and 43 minutes) of the predefined amount of time, a fourth bar portion indicating that the user's glucose range is “Low” or between 54 and 69 mg/dL for 3% (43 minutes) of the predefined amount of time, and a fifth bar portion indicating that the user's glucose range is “Very Low” or less than 54 mg/dL for 0% (0 minutes) of the predefined amount of time. As shown in FIG. 3F, according to some embodiments, Time-in-Ranges GUI 340 can display text adjacent to each bar portion indicating an actual amount of time, e.g., in hours and/or minutes. According to embodiments, the numerical values associated with the five bars can be adjusted for a user's personalized glucose target range.

According to one aspect of the embodiment shown in FIG. 3F, each bar portion of Time-in-Ranges GUI 340 can comprise a different color. In some embodiments, bar portions can be separated by dashed or dotted lines 342 and/or interlineated with numeric markers 344 to indicate the ranges reflected by the adjacent bar portions. In some embodiments, the time in ranges reflected by the bar portions can be further expressed as a percentage, an actual amount of time (e.g., 4 hours and 19 minutes), or, as shown in FIG. 3F, both. Furthermore, those of skill in the art will recognize that the percentages of time associated with each bar portion can vary depending on the analyte data of the user. In some embodiments of Time-in-Ranges GUI 340, the Target Range can be configured by the user. In other embodiments, the Target Range of Time-in-Ranges GUI 340 is not modifiable by the user. Furthermore, in addition to the numerical markers 344, the Time-in-Ranges GUI 340 may include target goals (e.g., “Goal: >70%” for “Target” Time-in-Range), which may be preset or user defined. The GUI 340 may also include text prompts which provide guidance to a user related to benefits or negative effects of remaining in certain ranges.

### Example Embodiments of Analyte Level and Trend Alert Interfaces

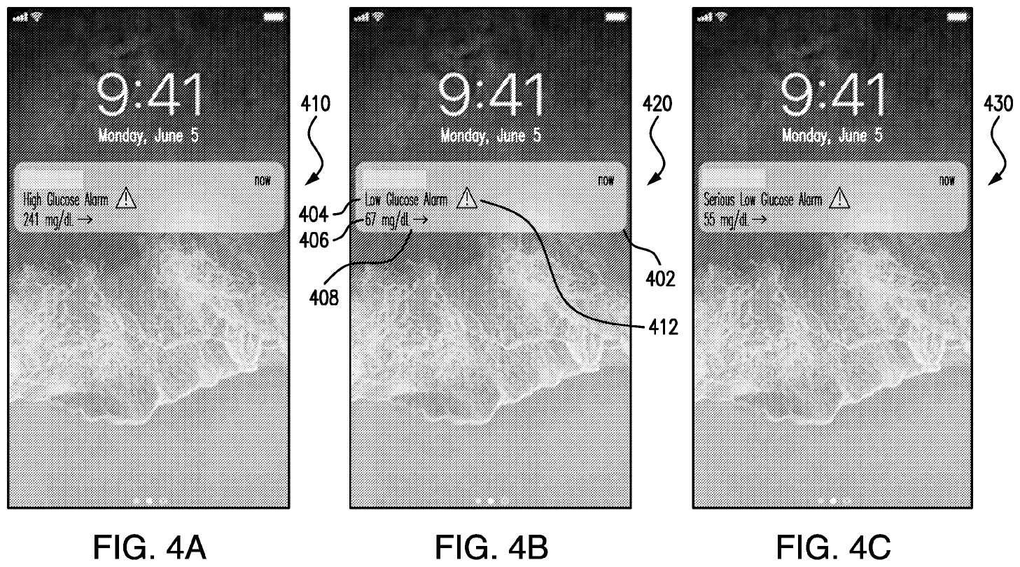

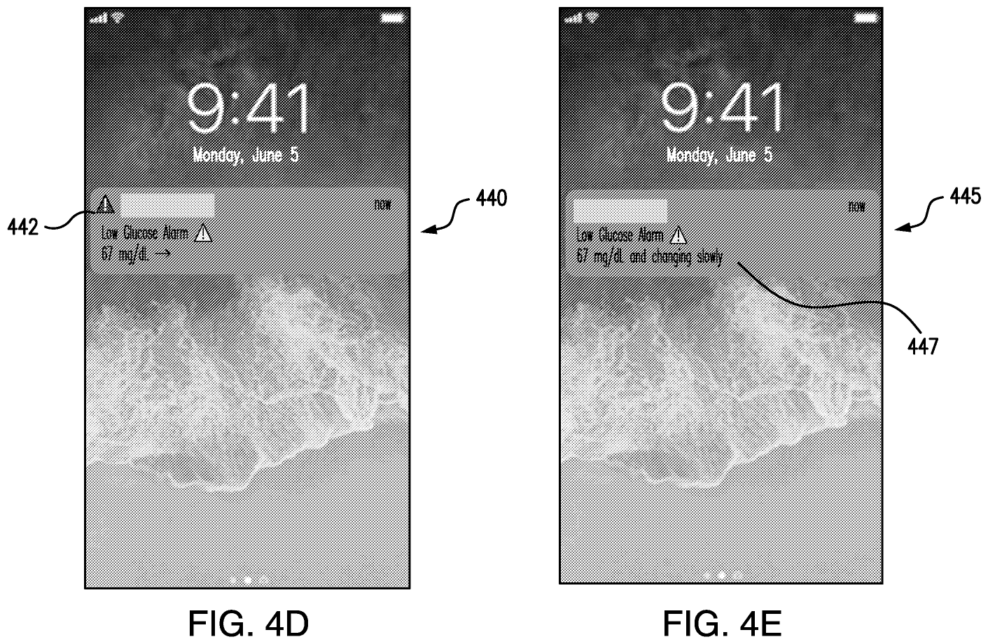

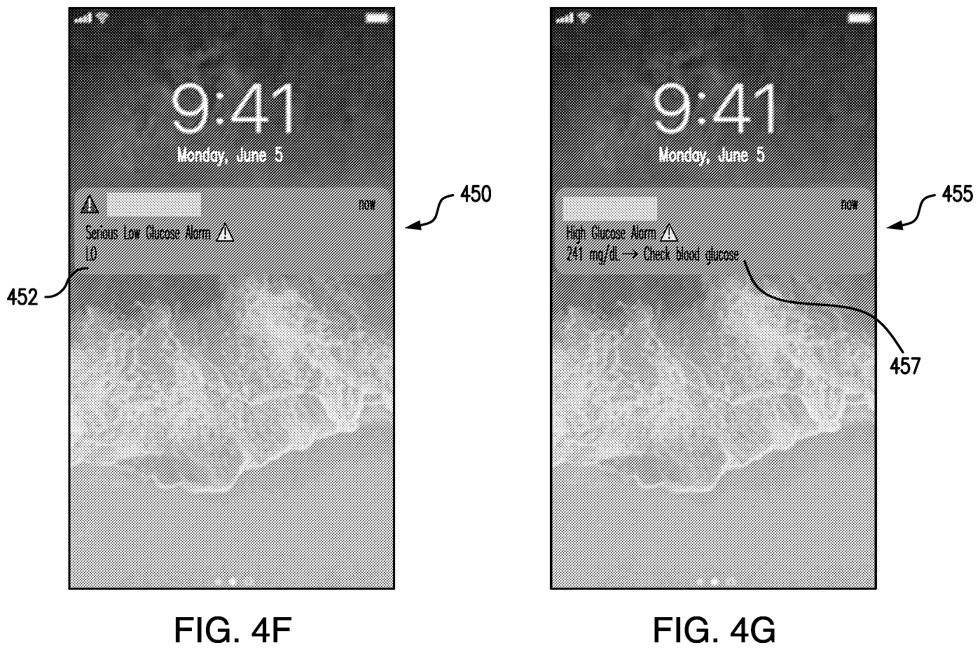

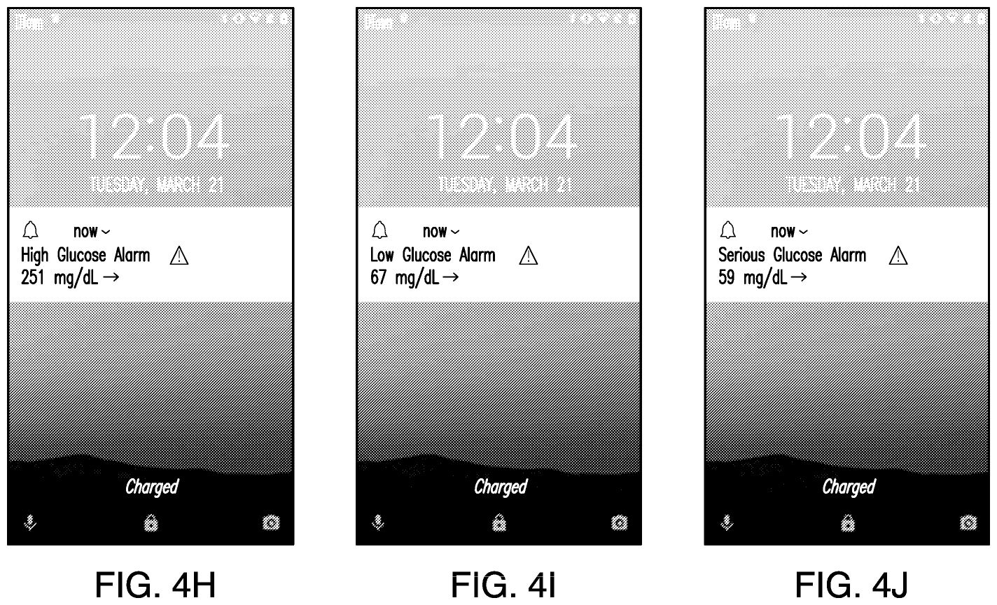

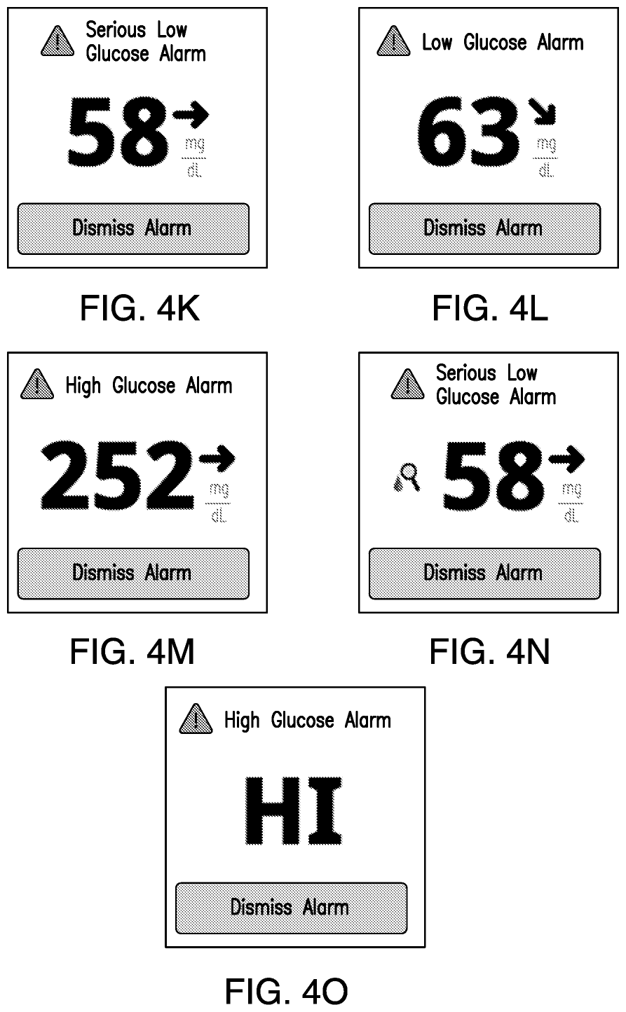

FIGS. 4A to 4O depict example embodiments of Analyte Level/Trend Alert GUIs for analyte monitoring systems. In accordance with the disclosed subject matter, the Analyte Level/Trend Alert GUIs comprise an audio or a visual notification (e.g., prompt, alert, alarm, pop-up window, banner notification, etc.), wherein the visual notification includes an alarm condition, an analyte level measurement associated with the alarm condition, and a trend indicator associated with the alarm condition. According to embodiment, at least one processor is configured to output a notification if at least one of the plurality of personalized glucose metrics is at or above the corresponding plurality of personalized glucose target. Notification can include an audio or a visual notification (e.g., prompt, alert, alarm, pop-up window, banner notification, etc.).

Turning to FIGS. 4A to 4C, example embodiments of a High Glucose Alarm 410, Low Glucose Alarm 420, and a Serious Low Glucose Alarms 430 are depicted, respectively, wherein each alarm comprises a pop-up window 402 containing an alarm condition text 404 (e.g., “Low Glucose Alarm”), an analyte level measurement 406 (e.g., a current glucose level of 67 mg/dL) associated with the alarm condition, and a trend indicator 408 (e.g., a trend arrow or directional arrow) associated with the alarm condition. In some embodiments, an alarm icon 412 can be adjacent to the alarm condition text 404. According to embodiments, analyte level measurement 406 can include a personalized analyte level measurement (e.g., a current personalized glucose level of 67 mg/dL).

Referring next to FIGS. 4D to 4G, additional example embodiments of Low Glucose Alarms 440, 445, Serious Low Glucose Alarm 450, and High Glucose Alarm 455 are depicted, respectively. As shown in FIG. 4D, Low Glucose Alarm 440 is similar to the Low Glucose Alarm of FIG. 4B (e.g., comprises a pop-up window containing an alarm condition text, an analyte level measurement associated with the alarm condition, and a trend indicator associated with the alarm condition), but further includes an alert icon 442 to indicate that the alarm has been configured as an alert (e.g., will display, play a sound, vibrate, even if the device is locked or if the device's “Do Not Disturb” setting has been enabled). With respect to FIG. 4E, Low Glucose Alarm 445 is also similar to the Low Glucose Alarm of FIG. 4B, but instead of including a trend arrow, Log Glucose Alarm 445 includes a textual trend indicator 447. According to one aspect of some embodiments, textual trend indicator 447 can be enabled through a device's Accessibility settings such that the device will “read” the textual trend indicator 447 to the user via the device's text-to-speech feature (e.g., Voiceover for iOS or Select-to-Speak for Android).

Referring next to FIG. 4F, Low Glucose Alarm 450 is similar to the Low Glucose Alarm of FIG. 4D (including the alert icon), but instead of displaying an analyte level measurement associated with an alarm condition and a trend indicator associated with the alarm condition, Low Glucose Alarm 450 displays a out-of-range indicator 452 to indicate that the current glucose level is either above or below a predetermined reportable analyte level range (e.g., “HI” or “LO”). According to embodiments, the current glucose level can include a current personalized glucose level, and the predetermined reportable analyte level range can include a predetermined reportable personalized analyte level range. With respect to FIG. 4G, High Glucose Alarm 455 is similar to the High Glucose Alarm of FIG. 4A (e.g., comprises a pop-up window containing an alarm condition text, an analyte level measurement associated with the alarm condition, and a trend indicator associated with the alarm condition), but further includes an instruction to the user 457. In some embodiments, for example, the instruction can be a prompt for the user to “Check blood glucose.” Those of skill in the art will appreciate that other instructions or prompts can be implemented (e.g., administer a corrective bolus, eat a meal, etc.).

Furthermore, although FIGS. 4A to 4G depict example embodiments of Analyte Level/Trend Alert GUIs that are displayed on smart phones having an iOS operating system, those of skill in the art will also appreciate that the Analyte Level/Trend Alert GUIs can be implemented on other devices including, e.g., smart phones with other operating systems, smart watches, wearables, reader devices, tablet computing devices, blood glucose meters, laptops, desktops, and workstations, to name a few. FIGS. 4H to 4J, for example, depict example embodiments of a High Glucose Alarm, Low Glucose Alarm, and a Serious Low Glucose Alarm for a smart phone having an Android Operating System. Similarly, FIGS. 4K to 4O depict, respectively, example embodiments of a Serious Low Glucose Alarm, Low Glucose Alarm, High Glucose Alarm, Serious Low Glucose Alarm (with a Check Blood Glucose icon), and High Glucose Alarm (with an out-of-range indicator) for a reader device.

### Example Embodiments of Sensor Usage Interfaces

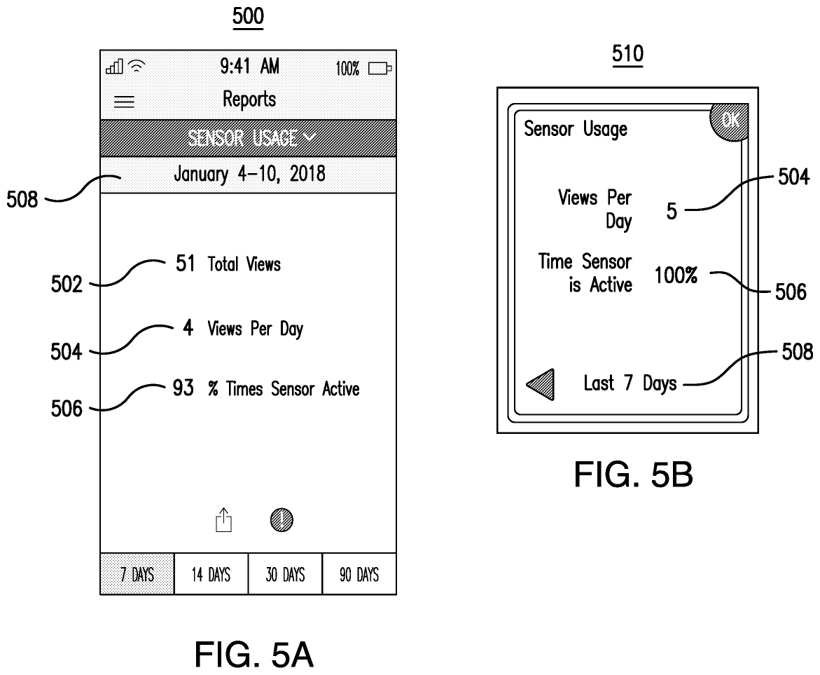

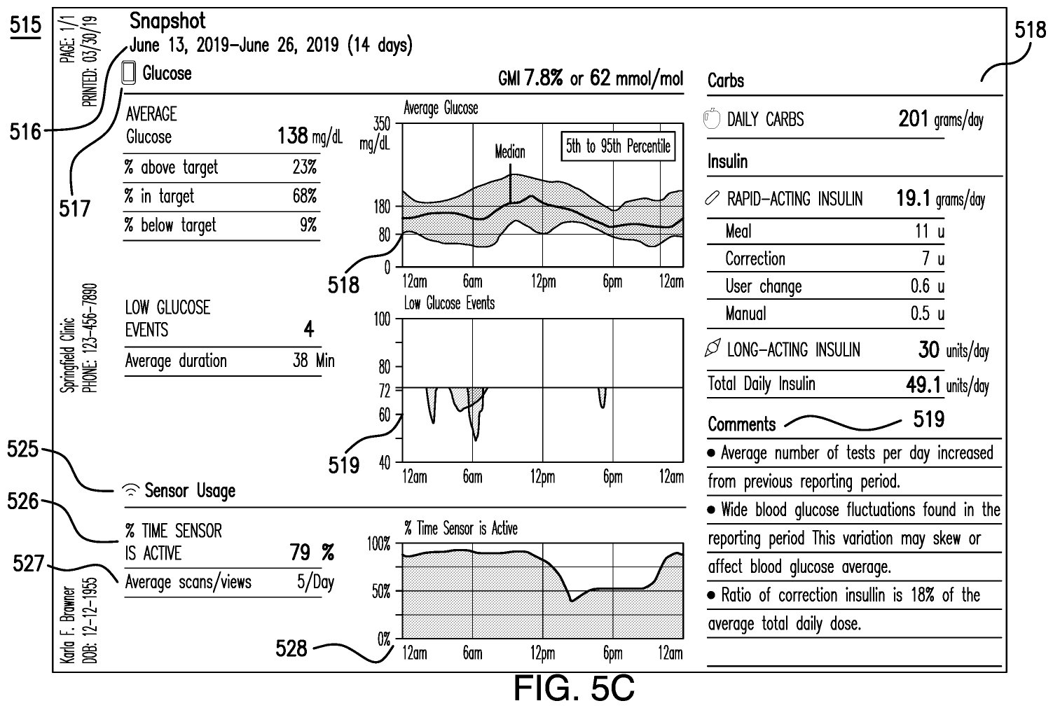

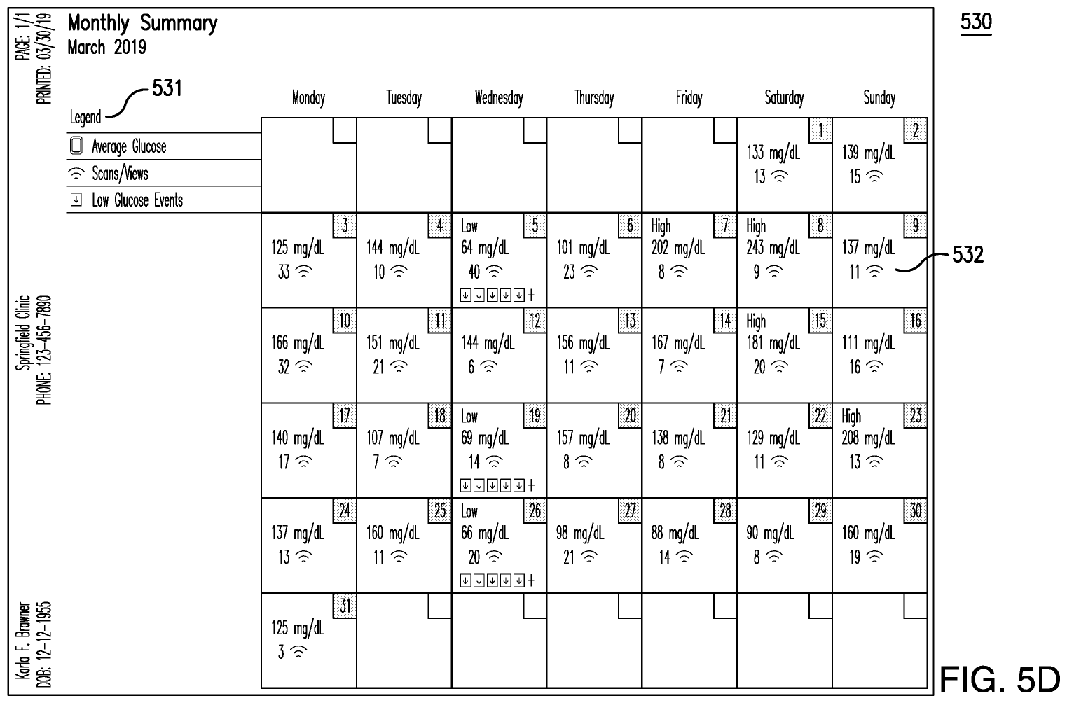

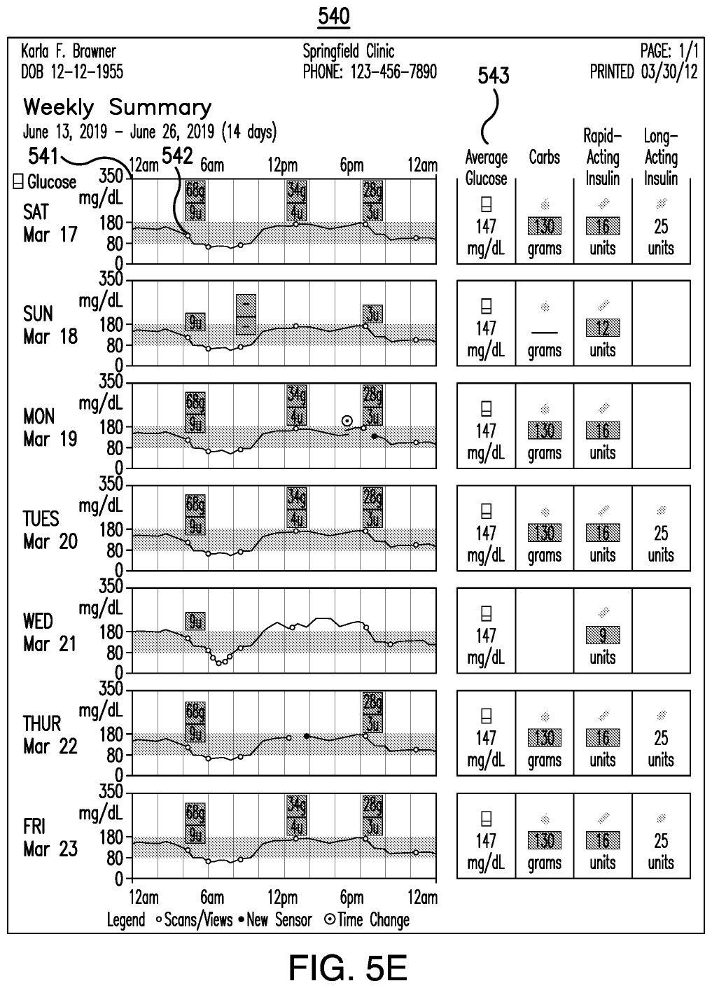

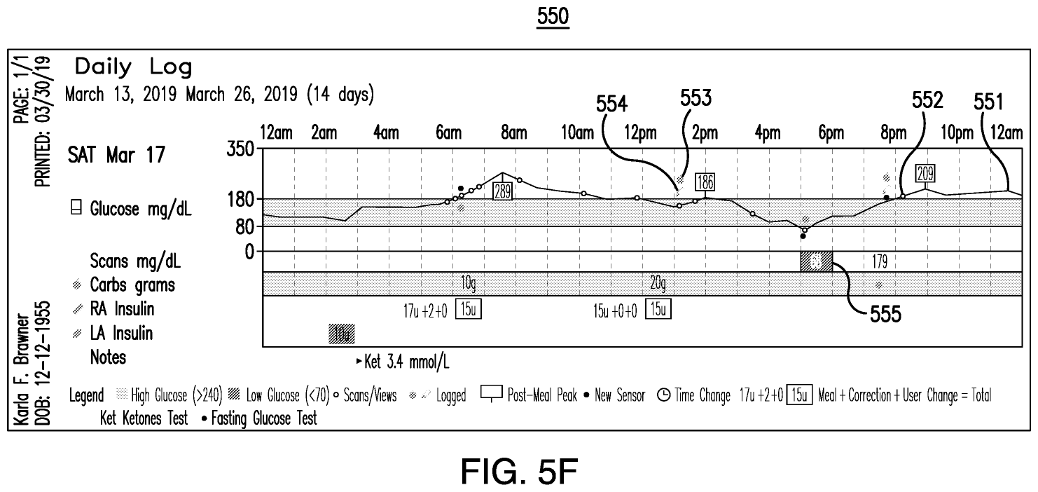

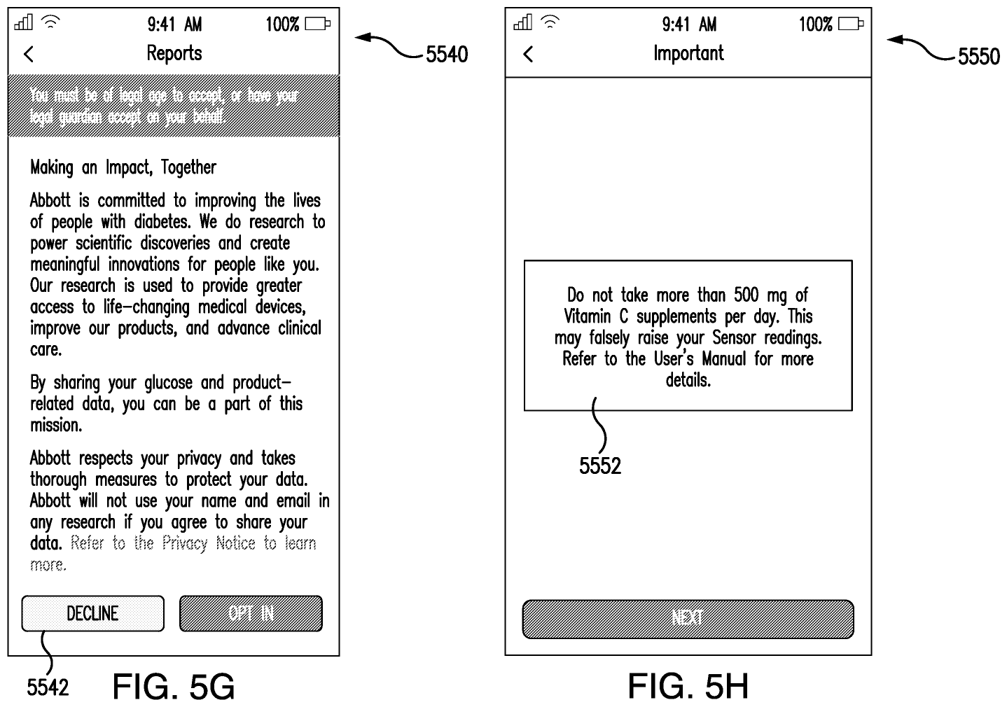

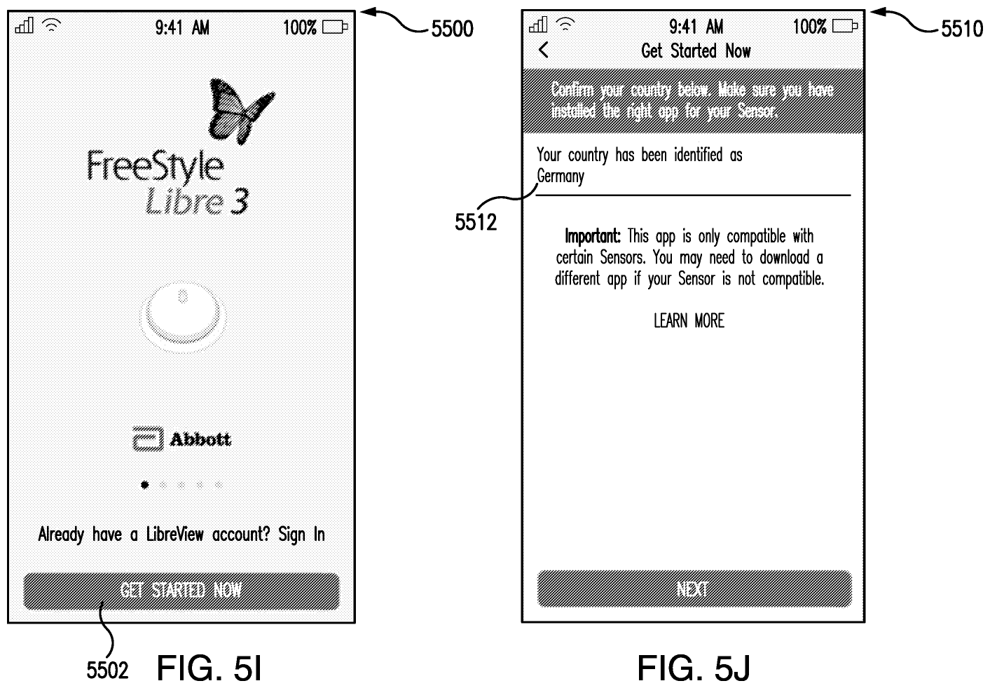

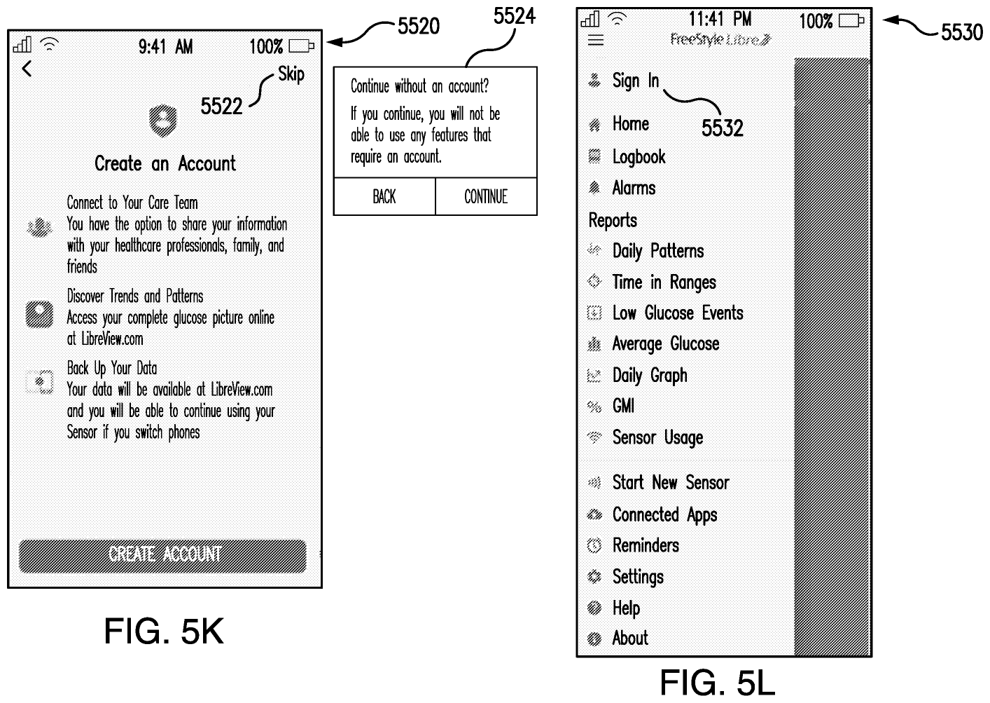

FIGS. 5A to 5F depict example embodiments of sensor usage interfaces relating to GUIs for analyte monitoring systems. In accordance with the disclosed subject matter, sensor usage interfaces provide for technological improvements including the capability to quantify and promote user engagement with analyte monitoring systems. For example, the user can benefit from subtle behavioral modification as the sensor usage interface encourages more frequent interaction with the device and the expected improvement in outcomes. The user can also benefit from increased frequent interaction which leads to improvement in a number of metabolic parameters, as discussed in further detail below.

In some embodiments, HCPs can receive a report of the user's frequency of interaction and a history of the patient's recorded metabolic parameters (e.g., estimated HbA1c levels, time in range of 70-180 mg/dL, etc.). If an HCP sees certain patients in their practice are less engaged than others, the HCPs can focus their efforts on improving engagement in users/patients that are less engaged than others. HCPs can benefit from more cumulative statistics (such as average glucose views per day, average glucose views before/after meals, average glucose views on “in-control” vs. “out-of-control” days or time of day) which may be obtained from the record of user's interaction frequency with the analyte monitoring systems and which can be used to understand why a patient may not be realizing expected gains from the analyte monitoring system. If an HCP sees that a patient is not benefiting as expected from the analyte monitoring system, they may recommend an increased level of interaction (e.g., increase interaction target level). Accordingly, an HCP can change the predetermined target level of interaction.

In some embodiments, caregivers can receive a report of the user's frequency of interaction. In turn, caregivers may be able to nudge the user to improve interaction with the analyte monitoring system. The caregivers may be able to use the data to better understand and improve their level of engagement with the user's analyte monitoring systems or alter therapy decisions.

According to some embodiments, for example, a sensor usage interface can include the visual display of one or more “view” metrics, each of which can be indicative of a measure of user engagement or interaction with the analyte monitoring system. A “view” can comprise, for example, an instance in which a sensor results interface is rendered or brought into the foreground (e.g., in certain embodiments, to view any of the GUI described herein). In some embodiments, the update interval as described above, data on sensor results GUI 245 is automatically updated or refreshed according to an update interval (e.g., every second, every minute, every 5 minutes, etc.). As such, a “view” can comprise one instance per update interval in which a sensor results interface is rendered or brought into the foreground. For example, if the update interval is every minute, rendering or bringing into the foreground the sensor results GUI 245 several times in that minute would only comprise one “view.” Similarly, if the sensor results GUI 245 is rendered or brought into the foreground for 20 continuous minutes, data on the senor results GUI 245 would be updated 20 times (i.e., once every minute). However, this would only constitute 20 “views” (i.e., one “view” per update interval). Similarly, if the update interval is every five minutes, rendering or bringing into the foreground the sensor results GUI 245 several times in those five minutes would only comprise one “view.” If the sensor results interface is rendered or brought into the foreground for 20 continuous minutes, this would constitute 4 “views” (i.e., one “view” each for each of the four five-minute intervals). According to other embodiments, a “view” can be defined as an instance when a user views a sensor results interface with a valid sensor reading for the first time in a sensor lifecount. According to disclosed embodiments, user can receive a notification, as described below, indicating when an instance of rendering or brining into the foreground the sensor results GUI is not counted as a “view.” For example, the user can receive a visual notification indicating such as “Results have not updated,” or “View does not count,” or “Please check glucose level again.” In some embodiments, the user can receive a check-in for each instance which counts as a “view,” as described in greater detail below.

According to disclosed embodiments, the one or more processors can be configured to record no more than one instance of user operation of the reader device during a defined time period. For example, and not limitation, a defined time period can include an hour. A person of ordinary skill in the art would understand defined time period to include any appropriate period of time, such as, one hour, two hours, three hours, 30 minutes, 15 minutes, etc.

According to some embodiments, a “view” can comprise, for example, a visual notification (e.g., prompt, alert, alarm, pop-up window, banner notification, etc.). In some embodiments, the visual notification can include an alarm condition, an analyte level measurement associated with the alarm condition, and a trend indicator associated with the alarm condition. For example, Analyte Level/Trend Alert GUIs, such as those embodiments depicted in FIGS. 4A to 4O can constitute a “view.”

In some embodiments, a sensor user interface can include a visual display of a “scan” metric indicative of another measure of user engagement or interaction with the analyte monitoring system. A “scan” can comprise, for example, an instance in which a user uses a reader device (e.g., smart phone, dedicated reader, etc.) to scan a sensor control device, such as, for example, in a Flash Analyte Monitoring system. As described above in connection with “views”, a “scan” can comprise one instance per update interval in a user uses a reader device to scan a sensor control device.

FIGS. 5A and 5B depict example embodiments of sensor usage interfaces 500 and 510, respectively. In accordance with the disclosed subject matter, sensor usage interfaces 500 and 510 can be rendered and displayed, for example, by a mobile app or software residing in non-transitory memory of reader device 120, such as those described with respect to FIGS. 1 and 2A. In some embodiments, for each instance of a “views” or “scans,” the software can record the date and time of the user's interaction with the system. In some embodiments, for each instance of a “view” or “scan,” the software can record the current glucose value. Referring to FIG. 5A, sensor user interface 500 can comprise: a predetermined time period interval 508 indicative of a time period (e.g., a date range) during which view metrics are measured, a Total Views metric 502, which is indicative of a total number of views over the predetermined time period 508; a Views Per Day metric 504, which is indicative of an average number of views per day over the predetermined time period 508; and a Percentage Time Sensor Active metric 506, which is indicative of the percentage of predetermined time period 508 that reader device 120 is in communication with sensor control device 102, such as those described with respect to FIGS. 1, 2B, and 2C. Referring to FIG. 5B, sensor user interface 510 can comprise a Views per Day metric 504 and a Percentage Time Sensor Active metric 508, each of which is measured for predetermined time period 508.

According to another aspect of the embodiments, although predetermined time period 508 is shown as one week, those of skill in the art will recognize that other predetermined time periods (e.g., 3 days, 14 days, 30 days) can be utilized. In addition, predetermined time period 508 can be a discrete period of time—with a start date and an end date—as shown in sensor usage interface 500 of FIG. 5A, or can be a time period relative to a current day or time (e.g., “Last 7 Days,” “Last 14 Days,” etc.), as shown in sensor usage interface 510 of FIG. 5B.

FIG. 5C depicts an example embodiment of sensor usage interface 525, as part of analyte monitoring system report GUI 515. In accordance with the disclosed subject matter, GUI 515 is a snapshot report covering a predetermined time period 516 (e.g., 14 days), and comprising a plurality of report portions on a single report GUI, including: a sensor usage interface portion 525, a glucose trend interface 517, which can include an glucose trend graph, a low glucose events graph, and other related glucose metrics (e.g., Glucose Management Indicator); a health information interface 518, which can include information logged by the user about the user's average daily carbohydrate intake and medication dosages (e.g., insulin dosages); and a comments interface 519, which can include additional information about the user's analyte and medication patterns presented in a narrative format. According to embodiments, health information interface 518 can include a graphical representation of average glucose level over a day relative to the foregoing target glucose range (shown with horizontal lines at 80 and 180 mg/dL). Glucose trend interface 517 can also include a percentage of Personalized A1C and/or a percent of Glucose Variability. In some embodiments, health information interface 518 can be segmented to indicate which range a user is in. For example, in some embodiments, the segmentation can be according to color. In particular, a low glucose range can be red, a good glucose range can be green, a high glucose range can be yellow, and very high glucose range can be orange; however, one having skill in the art will understand that different means for segmentation may also be possible. According to embodiments, segmentations may be defined by a user or a health care provider. According to embodiments, health information interface 518 can include a personalized-target glucose range report, such as those disclosed in International Publication No. WO2020/086934 to Xu, which is incorporated by reference in its entirety herein. According to embodiments, the personalized-target glucose range report can include a graphical representation of glucose level over a day relative to the foregoing personalized-target glucose range. According to another aspect of the embodiments, sensor usage interface 525 can comprise a Percentage Time Sensor Active metric 526, an Average Scans/Views metric 527 (e.g., indicative of an average sum of a number of scans and a number of views), and a Percentage Time Sensor Active graph 528. As can be seen in FIG. 5C, an axis of the Percentage Time Sensor Active graph can be aligned with a corresponding axis of one or more other graphs (e.g., average glucose trend graph, low glucose events graph), such that the user can visually correlate data between multiple graphs from two or more portions of the report GUI by the common units (e.g., time of day) from the aligned axes.

FIG. 5D depicts an example embodiment of another analyte monitoring system report GUI 530 including sensor usage information. In accordance with the disclosed subject matter, GUI 530 is a monthly summary report including a first portion comprising a legend 531, wherein legend 531 includes a plurality of graphical icons each of which is adjacent to a descriptive text. As shown in FIG. 5D, legend 531 includes an icon and descriptive text for “Average Glucose,” an icon and descriptive text for “Scans/Views,” and an icon and descriptive text for “Low Glucose Events.” GUI 530 also includes a second portion comprising a calendar interface 532. For example, as shown in FIG. 5D, GUI 530 comprises a monthly calendar interface, wherein each day of the month can include one or more of an average glucose metric, low glucose event icons, and a sensor usage metric 532. In some embodiments, such as the one shown in FIG. 5D, the sensor usage metric (“scans/views”) is indicative of a total sum of a number of scans and a number of views for each day. According to embodiments, an average glucose metric can include a personalized average glucose metric.

FIG. 5E depicts an example embodiment of another analyte monitoring system report GUI 540 including sensor usage information. In accordance with the disclosed subject matter, GUI 540 is a weekly summary report including a plurality of report portions, wherein each report portion is representative of a different day of the week, and wherein each report portion comprises a glucose trend graph 541, which can include the user's measured glucose levels over a twenty-four hour period, and a health information interface 543, which can include information about the user's average daily glucose, carbohydrate intake, and/or insulin dosages. In some embodiments, glucose trend graph 541 can include sensor usage markers 542 to indicate that a scan, a view, or both had occurred at a particular time during the twenty-four hour period. According to embodiments, glucose trend graph 541 can include the user's personalized glucose levels over a twenty-four hour period. According to embodiments, glucose trend graph 541 can include a personalized-target average glucose report, which can include a graphical representation of a subject's average glucose (for example, not limitation, shown by a solid line) over time and the personalized-target average glucose. According to embodiments, health information interface 543 can include information about the user's personalized average daily glucose.

FIG. 5F depicts an example embodiment of another analyte monitoring system report GUI 550 including sensor usage information. In accordance with the disclosed subject matter, GUI 550 is a daily log report comprising a glucose trend graph 551, which can include the user's glucose levels over a twenty-four hour period. According to embodiments, glucose trend graph 541 can include the user's personalized glucose levels over a twenty-four hour period. In some embodiments, glucose trend graph 551 can include sensor usage markers 552 to indicate that a scan, a view, or both had occurred at a particular time during the twenty-four hour period. Glucose trend graph 551 can also include logged event markers, such as logged carbohydrate intake markers 553 and logged insulin dosage markers 554, as well as glucose event markers, such as low glucose event markers 555.

According to embodiments, FIGS. 5A-F could additionally include laboratory measured HbA1c (“Lab A1c”).

FIGS. 5I to 5L depict various GUIs for improving usability and user privacy with respect to analyte monitoring software. FIG. 5G, GUI 5540 depicts a research consent interface 5540, which prompts the user to choose to either decline or opt in (through buttons 5542) with respect to permitting the user's analyte data and/or other product-related data to be used for research purposes. According to embodiments of the disclosed subject matter, the analyte data can be anonymized (de-identified) and stored in an international database for research purposes.

Referring next to FIG. 5H, GUI 5550 depicts a “Vitamin C” warning interface 5550 which displays a warning to the user that the daily use of more than 500 mg of Vitamin C supplements can result in falsely high sensor readings.

FIG. 5I is GUI 5500 depicting a first start interface which can be displayed to a user the first time the analyte monitoring software is started. In accordance with the disclosed subject matter, GUI 5500 can include a “Get Started Now” button 5502 that, when pressed, will navigate the user to GUI 5510 of FIG. 5J. GUI 5510 depicts a country confirmation interface 5512 that prompts the user to confirm the user's country. According to another aspect of the embodiments, the country selected can limit and/or enable certain interfaces within the analyte monitoring software application for regulatory compliance purposes.

Turning next to FIG. 5K, GUI 5520 depicts a user account creation interface which allows the user to initiate a process to create a cloud-based user account. In accordance with the disclosed subject matter, a cloud-based user account can allow the user to share information with healthcare professionals, family and friends; utilize a cloud-based reporting platform to review more sophisticated analyte reports; and back up the user's historical sensor readings to a cloud-based server. In some embodiments, GUI 5520 can also include a “Skip” link 5522 that allows a user to utilize the analyte monitoring software application in an “accountless mode” (e.g., without creating or linking to a cloud-based account). Upon selecting the “Skip” link 5522, an information window 5524 can be displayed to inform that certain features are not available in “accountless mode.” Information window 5524 can further prompt the user to return to GUI 5520 or proceed without account creation.

FIG. 5L is GUI 5530 depicting a menu interface displayed within an analyte monitoring software application while the user is in “accountless mode.” According to an aspect of the embodiments, GUI 5530 includes a “Sign in” link 5532 that allows the user to leave “accountless mode” and either create a cloud-based user account or sign-in with an existing cloud-based user account from within the analyte monitoring software application.

It will be understood by those of skill in the art that any of the GUIs, reports interfaces, or portions thereof, as described herein, are meant to be illustrative only, and that the individual elements, or any combination of elements, depicted and/or described for a particular embodiment or figure are freely combinable with any elements, or any combination of elements, depicted and/or described with respect to any of the other embodiments.

### Example Embodiments of Digital Interfaces for Analyte Monitoring Systems

Described herein are example embodiments of digital interfaces for analyte monitoring systems. In accordance with the disclosed subject matter, a digital interface can comprise a series of instructions, routines, subroutines, and/or algorithms, such as software and/or firmware stored in a non-transitory memory, executed by one or more processors of one or more devices in an analyte monitoring system, wherein the instructions, routines, subroutines, or algorithms are configured to enable certain functions and inter-device communications. As an initial matter, it will be understood by those of skill in the art that the digital interfaces described herein can comprise instructions stored in a non-transitory memory of a sensor control device 102, reader device 120, local computer system 170, trusted computer system 180, and/or any other device or system that is part of, or in communication with, analyte monitoring system 100, as described with respect to FIGS. 1, 2A, and 2B. These instructions, when executed by one or more processors of the sensor control device 102, reader device 120, local computer system 170, trusted computer system 180, or other device or system of analyte monitoring system 100, cause the one or more processors to perform the method steps described herein. Those of skill in the art will further recognize that the digital interfaces described herein can be stored as instructions in the memory of a single centralized device or, in the alternative, can be distributed across multiple discrete devices in geographically dispersed locations.

### Example Embodiments of Methods for Data Backfilling

Example embodiments of methods for data backfilling in an analyte monitoring system will now be described. In accordance with the disclosed subject matter, gaps in analyte data and other information can result from interruptions to communication links between various devices in an analyte monitoring system 100. These interruptions can occur, for example, from a device being powered off (e.g., a user's smart phone runs out of battery), or a first device temporarily moving out of a wireless communication range from a second device (e.g., a user wearing sensor control device 102 inadvertently leaves her smart phone at home when she goes to work). As a result of these interruptions, reader device 120 may not receive analyte data and other information from sensor control device 102. It would thus be beneficial to have a robust and flexible method for data backfilling in an analyte monitoring system to ensure that once a communication link is re-established, each analyte monitoring device can receive a complete set of data, as intended.

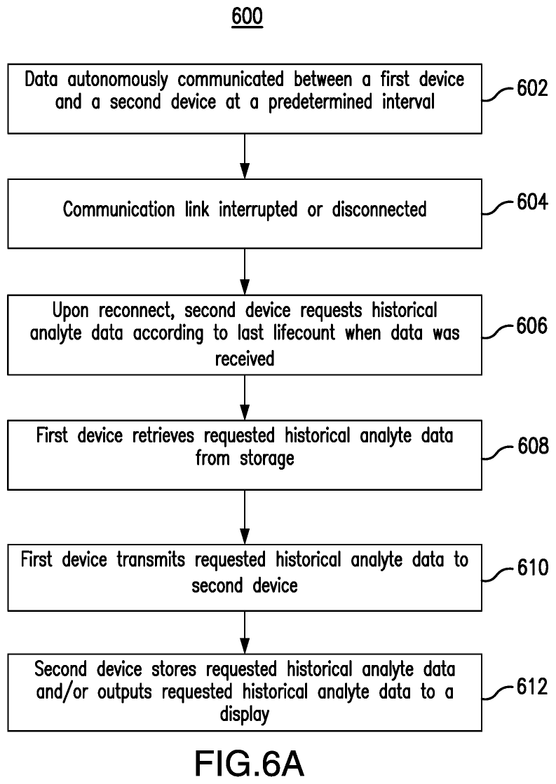

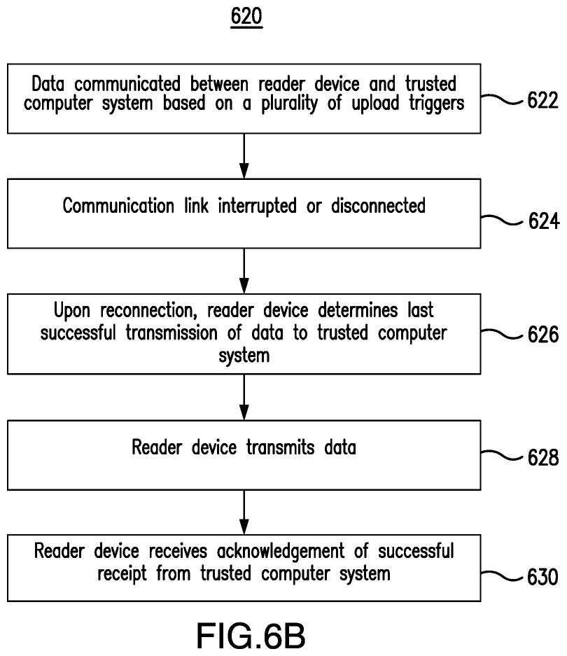

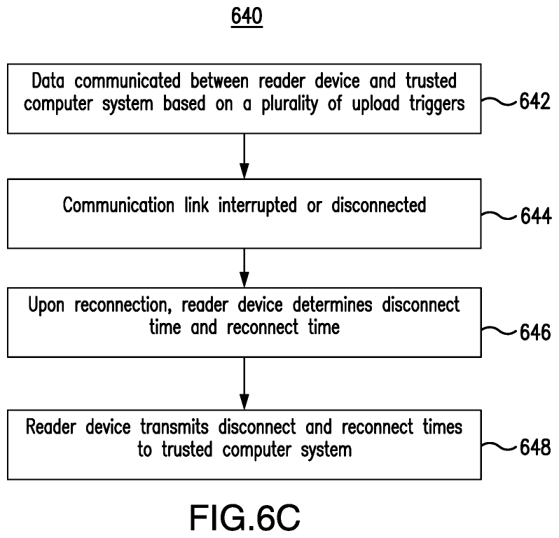

FIG. 6A is a flow diagram depicting an example embodiment of a method 600 for data backfilling in an analyte monitoring system. In accordance with the disclosed subject matter, method 600 can be implemented to provide data backfilling between a sensor control device 102 and a reader device 120. At Step 602, analyte data and other information is autonomously communicated between a first device and a second device at a predetermined interval. In some embodiments, the first device can be a sensor control device 102, and the second device can be a reader device 120, as described with respect to FIGS. 1, 2A, and 2B. In accordance with the disclosed subject matter, analyte data and other information can include, but is not limited to, one or more of: data indicative of an analyte level in a bodily fluid, a rate-of-change of an analyte level, a predicted analyte level, a low or a high analyte level alert condition, a sensor fault condition, or a communication link event. According to another aspect of the embodiments, autonomous communications at a predetermined interval can comprise streaming analyte data and other information according to a standard wireless communication network protocol, such as a Bluetooth or Bluetooth Low Energy protocol, at one or more predetermined rates (e.g., every minute, every five minutes, every fifteen minutes, etc.). In some embodiments, different types of analyte data or other information can be autonomously communicated between the first and second devices at different predetermined rates (e.g., historical glucose data every 5 minutes, current glucose value every minute, etc.).

At Step 604, a disconnection event or condition occurs that causes an interruption to the communication link between the first device and the second device. As described above, the disconnection event can result from the second device (e.g., reader device 120, smart phone, etc.) running out of battery power or being powered off manually by a user. A disconnection event can also result from the first device being moved outside a wireless communication range of the second device, from the presence of a physical barrier that obstructs the first device and/or the second device, or from anything that otherwise prevents wireless communications from occurring between the first and second devices.

At Step 606, the communication link is re-established between the first device and the second device (e.g., the first device comes back into the wireless communication range of the second device). Upon reconnection, the second device requests historical analyte data according to a last lifecount metric for which data was received. In accordance with the disclosed subject matter, the lifecount metric can be a numeric value that is incremented and tracked on the second device in units of time (e.g., minutes), and is indicative of an amount of time elapsed since the sensor control device was activated. For example, in some embodiments, after the second device (e.g., reader device 120, smart phone, etc.) re-establishes a Bluetooth wireless communication link with the first device (e.g., sensor control device 120), the second device can determine the last lifecount metric for which data was received. Then, according to some embodiments, the second device can send to the first device a request for historical analyte data and other information having a lifecount metric greater than the determined last lifecount metric for which data was received.

In some embodiments, the second device can send a request to the first device for historical analyte data or other information associated with a specific lifecount range, instead of requesting historical analyte data associated with a lifecount metric greater than a determined last lifecount metric for which data was received.

At Step 608, upon receiving the request, the first device retrieves the requested historical analyte data from storage (e.g., non-transitory memory of sensor control device 102), and subsequently transmits the requested historical analyte data to the second device at Step 610. At Step 612, upon receiving the requested historical analyte data, the second device stores the requested historical analyte data in storage (e.g., non-transitory memory of reader device 120). In accordance with the disclosed subject matter, when the requested historical analyte data is stored by the second device, it can be stored along with the associated lifecount metric. In some embodiments, the second device can also output the requested historical analyte data to a display of the second device, such as, for example to a glucose trend graph of a sensor results GUI, such as those described with respect to FIGS. 2D to 2I. For example, in some embodiments, the requested historical analyte data can be used to fill in gaps in a glucose trend graph by displaying the requested historical analyte data along with previously received analyte data.

Furthermore, those of skill in the art will appreciate that the method of data backfilling can be implemented between multiple and various devices in an analyte monitoring system, wherein the devices are in wired or wireless communication with each other.

FIG. 6B is a flow diagram depicting another example embodiment of a method 620 for data backfilling in an analyte monitoring system. In accordance with the disclosed subject matter, method 620 can be implemented to provide data backfilling between a reader device 120 (e.g., smart phone, dedicated reader) and a trusted computer system 180, such as, for example, a cloud-based platform for generating reports. At Step 622, analyte data and other information is communicated between reader device 120 and trusted computer system 180 based on a plurality of upload triggers. In accordance with the disclosed subject matter, analyte data and other information can include, but are not limited to, one or more of: data indicative of an analyte level in a bodily fluid (e.g., current glucose level, historical glucose data), a rate-of-change of an analyte level, a predicted analyte level, a low or a high analyte level alert condition, information logged by the user, information relating to sensor control device 102, alarm information (e.g., alarm settings), wireless connection events, and reader device settings, to name a few.

According to another aspect of the embodiments, the plurality of upload triggers can include (but is not limited to) one or more of the following: activation of sensor control device 102; user entry or deletion of a note or log entry; a wireless communication link (e.g., Bluetooth) reestablished between reader device 120 and sensor control device 102; alarm threshold changed; alarm presentation, update, or dismissal; internet connection re-established; reader device 120 restarted; a receipt of one or more current glucose readings from sensor control device 102; sensor control device 120 terminated; signal loss alarm presentation, update, or dismissal; signal loss alarm is toggled on/offt view of sensor results screen GUI; or user sign-in into cloud-based platform.

According to another aspect of the embodiments, in order to track the transmission and receipt of data between devices, reader device 120 can “mark” analyte data and other information that is to be transmitted to trusted computer system 180. In some embodiments, for example, upon receipt of the analyte data and other information, trusted computer system 180 can send a return response to reader device 120, to acknowledge that the analyte data and other information has been successfully received. Subsequently, reader device 120 can mark the data as successfully sent. In some embodiments, the analyte data and other information can be marked by reader device 120 both prior to being sent and after receipt of the return response. In other embodiments, the analyte data and other information can be marked by reader device 120 only after receipt of the return response from trusted computer system 180.

Referring to FIG. 6B, at Step 624, a disconnection event occurs that causes an interruption to the communication link between reader device 120 and trusted computer system 180. For example, the disconnection event can result from the user placing the reader device 120 into “airplane mode” (e.g., disabling of the wireless communication modules), from the user powering off the reader device 120, or from the reader device 120 moving outside of a wireless communication range.

At Step 626, the communication link between reader device 120 and trusted computer system 180 (as well as the internet) is re-established, which is one of the plurality of upload triggers. Subsequently, reader device 120 determines the last successful transmission of data to trusted computer system 180 based on the previously marked analyte data and other information sent. Then, at Step 628, reader device 120 can transmit analyte data and other information not yet received by trusted computer system 180. At Step 630, reader device 120 receives acknowledgement of successful receipt of analyte data and other information from trusted computer system 180.

Although FIG. 6B is described above with respect to a reader in communication with a trusted computer system, those of skill in the art will appreciate that the data backfilling method can be applied between other devices and computer systems in an analyte monitoring system (e.g., between a reader and a local computer system, between a reader and a medical delivery device, between a reader and a wearable computing device, etc.). These embodiments, along with their variations and permutations, are fully within the scope of this disclosure.

In addition to data backfilling, example embodiments of methods for aggregating disconnect and reconnect events for wireless communication links in an analyte monitoring system are described. In accordance with the disclosed subject matter, there can be numerous and wide-ranging causes for interruptions to wireless communication links between various devices in an analyte monitoring system. Some causes can be technical in nature (e.g., a reader device is outside a sensor control device's wireless communication range), while other causes can relate to user behavior (e.g., a user leaving his or her reader device at home). In order to improve connectivity and data integrity in analyte monitoring systems, it would therefore be beneficial to gather information regarding the disconnect and reconnect events between various devices in an analyte monitoring system.

FIG. 6C is a flow diagram depicting an example embodiment of a method 640 for aggregating disconnect and reconnect events for wireless communication links in an analyte monitoring system. In some embodiments, for example, method 640 can be used to detect, log, and upload to trusted computer system 180, Bluetooth or Bluetooth Low Energy disconnect and reconnect events between a sensor control device 102 and a reader device 120. In accordance with the disclosed subject matter, trusted computer system 180 can aggregate disconnect and reconnect events transmitted from a plurality of analyte monitoring systems. The aggregated data can then by analyzed to determine whether any conclusions can be made about how to improve connectivity and data integrity in analyte monitoring systems.

At Step 642, analyte data and other information are communicated between reader device 120 and trusted computer system 180 based on a plurality of upload triggers, such as those previously described with respect to method 620 of FIG. 6B. At Step 644, a disconnection event occurs that causes an interruption to the wireless communication link between sensor control device 102 and reader device 120. Example disconnection events can include, but are not limited to, a user placing the reader device 120 into “airplane mode,” the user powering off the reader device 120, the reader device 120 running out of power, the sensor control device 102 moving outside a wireless communication range of the reader devices 120, or a physical barrier obstructing the sensor control device 102 and/or the reader device 120, to name only a few.

Referring still to FIG. 6C, at Step 646, the wireless communication link between the sensor control device 102 and reader device 120 is re-established, which is one of the plurality of upload triggers. Subsequently, reader device 120 determines a disconnect time and a reconnect time, wherein the disconnect time is the time that the interruption to the wireless communication link began, and the reconnect time is the time that the wireless communication link between the sensor control device 102 and reader device 120 is re-established. According to some embodiments, the disconnection and reconnection times can also be stored locally in an event log on reader device 120. At Step 648, reader device 120 transmits the disconnect and reconnect times to trusted computer system 180.

According to some embodiments, the disconnect and reconnect times can be stored in non-transitory memory of trusted computer system 180, such as in a database, and aggregated with the disconnect and reconnect times collected from other analyte monitoring systems. In some embodiments, the disconnect and reconnect times can also be transmitted to and stored on a different cloud-based platform or server from trusted computer system 180 that stores analyte data. In still other embodiments, the disconnect and reconnect times can be anonymized.

In addition, those of skill in the art will recognize that method 640 can be utilized to collect disconnect and reconnect times between other devices in an analyte monitoring system, including, for example: between reader device 120 and trusted computer system 180; between reader device 120 and a wearable computing device (e.g., smart watch, smart glasses); between reader device 120 and a medication delivery device (e.g., insulin pump, insulin pen); between sensor control device 102 and a wearable computing device; between sensor control device 102 and a medication delivery device; and any other combination of devices within an analyte monitoring system. Those of skill in the art will further appreciate that method 640 can be utilized to analyze disconnect and reconnect times for different wireless communication protocols, such as, for example, Bluetooth or Bluetooth Low Energy, NFC, 802.11x, UHF, cellular connectivity, or any other standard or proprietary wireless communication protocol.

### Example Embodiments of Improved Expired/Failed Sensor Transmissions

Example embodiments of methods for improved expired and/or failed sensor transmissions in an analyte monitoring system will now be described. In accordance with the disclosed subject matter, expired or failed sensor conditions detected by a sensor control device 102 can trigger alerts on reader device 120. However, if the reader device 120 is in “airplane mode,” powered off, outside a wireless communication range of sensor control device 102, or otherwise unable to wirelessly communicate with the sensor control device 102, then the reader device 120 may not receive these alerts. This can cause the user to miss information such as, for example, the need to promptly replace a sensor control device 102. Failure to take action on a detected sensor fault can also lead to the user being unaware of adverse glucose conditions (e.g., hypoglycemia and/or hyperglycemia) due to a terminated sensor.

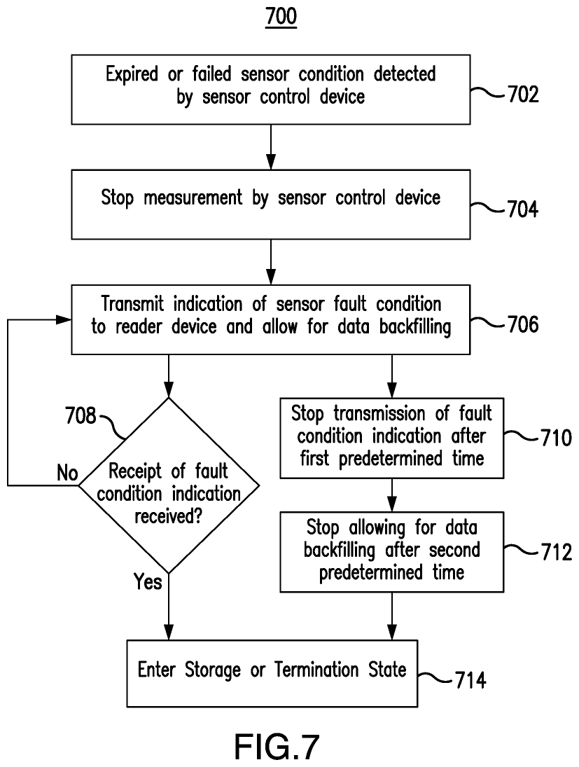

FIG. 7 is a flow diagram depicting an example embodiment of a method 700 for improved expired or failed sensor transmissions in an analyte monitoring system. In accordance with the disclosed subject matter, method 700 can be implemented to provide for improved sensor transmissions by a sensor control device 102 after an expired or failed sensor condition has been detected. At Step 702, an expired or failed sensor condition is detected by sensor control device 102. In some embodiments, the sensor fault condition can comprise one or both of a sensor insertion failure condition or a sensor termination condition. According to some embodiments, for example, a sensor insertion failure condition or a sensor termination condition can include, but is not limited to, one or more of the following: a FIFO overflow condition detected, a sensor signal below a predetermined insertion failure threshold, moisture ingress detected, an electrode voltage exceeding a predetermined diagnostic voltage threshold, an early signal attenuation (ESA) condition, or a late signal attenuation (LSA) condition, to name a few.

Referring again to FIG. 7, at Step 704, sensor control device 102 stops acquiring measurements of analyte levels from the analyte sensor in response to the detection of the sensor fault condition. At Step 706, sensor control device 102 begins transmitting an indication of a sensor fault condition to reader device 120, while also allowing for the reader device 120 to connect to the sensor control device 102 for purposes of data backfilling. In accordance with the disclosed subject matter, the transmission of the indication of the sensor fault condition can comprise transmitting a plurality of Bluetooth or Bluetooth Low Energy advertising packets, each of which can include the indication of the sensor fault condition. In some embodiments, the plurality of Bluetooth or BLE advertising packets can be transmitted repeatedly, continuously, or intermittently. Those of skill in the art will recognize that other modes of wirelessly broadcasting or multicasting the indication of the sensor fault condition can be implemented. According to another aspect of the embodiments, in response to receiving the indication of the sensor fault condition, reader device 120 can visually display an alert or prompt for a confirmation by the user.

At Step 708, sensor control device 102 can be configured to monitor for a return response or acknowledgment of receipt of the indication of the sensor fault condition from reader device 120. In some embodiments, for example, a return response or acknowledgement of receipt can be generated by reader device 120 when a user dismisses an alert on the reader device 120 relating to the indication of the sensor fault condition, or otherwise responds to a prompt for confirmation of the indication of the sensor fault condition. If a return response or acknowledgement of receipt of the indication of the sensor fault condition is received by sensor control device 102, then at Step 714, sensor control device 102 can enter either a storage state or a termination state. According to some embodiments, in the storage state, the sensor control device 102 is placed in a low-power mode, and the sensor control device 102 is capable of being re-activated by a reader device 120. By contrast, in the termination state, the sensor control device 102 cannot be re-activated and must be removed and replaced.

If a receipt of the fault condition indication is not received by sensor control device 102, then at Step 710, the sensor control device 102 will stop transmitting the fault condition indication after a first predetermined time period. In some embodiments, for example, the first predetermined time period can be one of: one hour, two hours, five hours, etc. Subsequently, at Step 712, if a receipt of the fault condition indication is still not received by sensor control device 102, then at Step 712, the sensor control device 102 will also stop allowing for data backfilling after a second predetermined time period. In some embodiments, for example, the second predetermined time period can be one of: twenty-four hours, forty-eight hours, etc. Sensor control device 102 then enters a storage state or a termination state at Step 714.

By allowing sensor control device 102 to continue transmissions of sensor fault conditions for a predetermined time period, the embodiments of this disclosure mitigate the risk of unreceived sensor fault alerts. In addition, although the embodiments described above are in reference to a sensor control device 102 in communication with a reader device 120, those of skill in the art will recognize that indications of sensor fault conditions can also be transmitted between a sensor control device 102 and other types of mobile computing devices, such as, for example, wearable computing devices (e.g., smart watches, smart glasses) or tablet computing devices.

### Example Embodiments of Data Merging in Analyte Monitoring Systems

Example embodiments of methods for merging data received from one or more analyte monitoring systems will now be described. As described earlier with respect to FIG. 1, a trusted computer system 180, such as a cloud-based platform, can be configured to generate various reports based on received analyte data and other information from a plurality of reader devices 120 and sensor control devices 102. A large and diverse population of reader devices and sensor control devices, however, can give rise to complexities and challenges in generating reports based on the received analyte data and other information. For example, a single user may have multiple reader devices and/or sensor control devices, either simultaneously or serially over time, each of which can comprise different versions. This can lead to further complications in that, for each user, there may be sets of duplicative and/or overlapping data. It would therefore be beneficial to have methods for merging data at a trusted computer system for purposes of report generation.

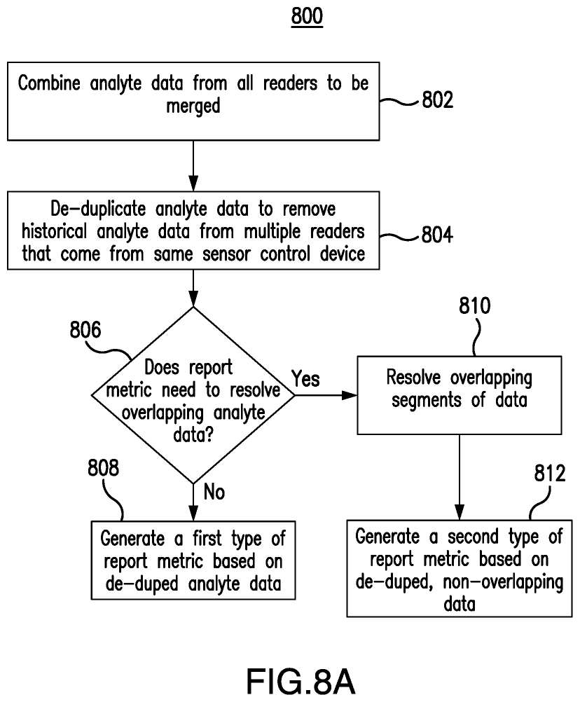

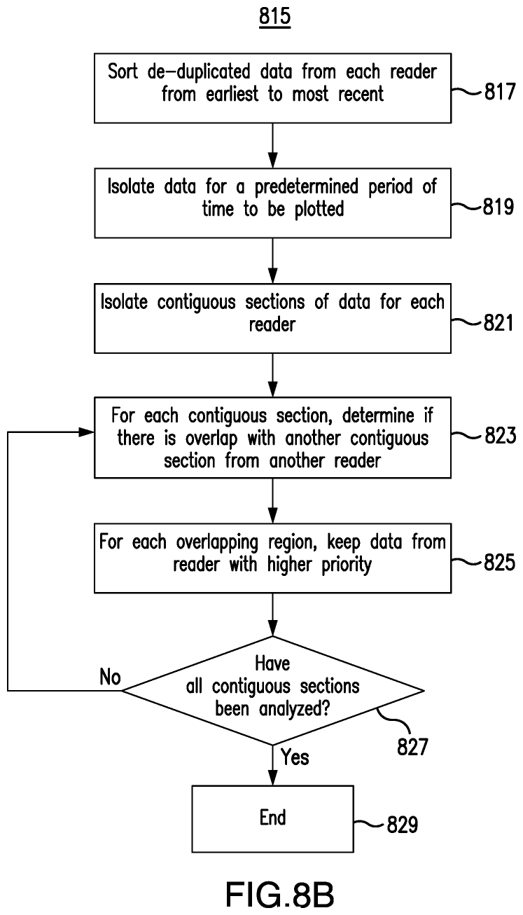

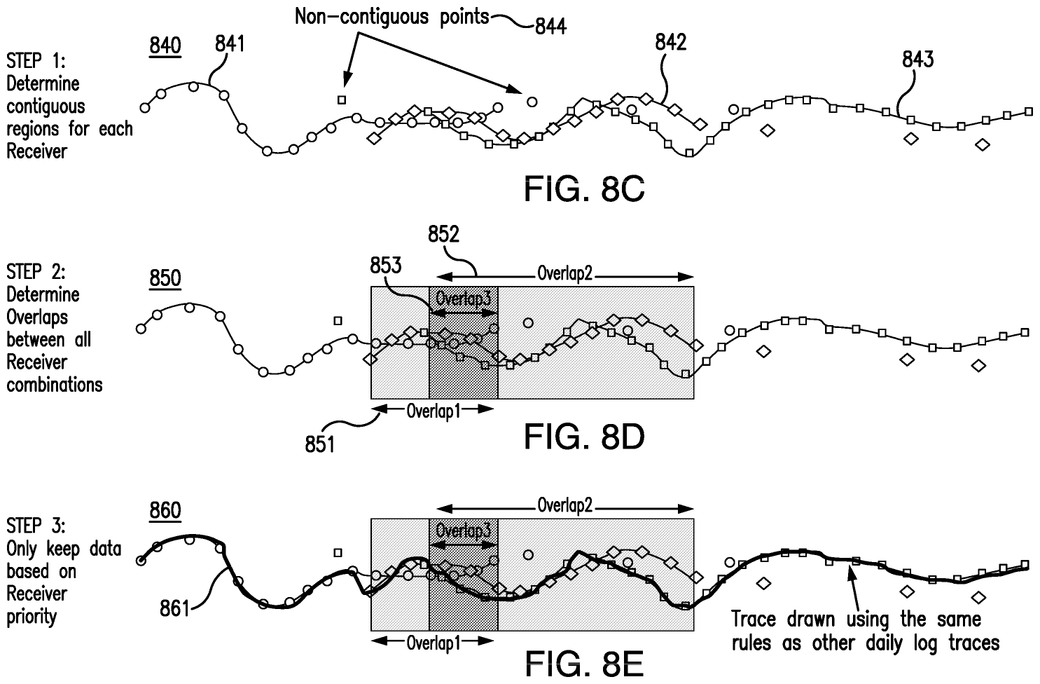

FIG. 8A is a flow diagram depicting an example embodiment of a method 800 for merging data associated with a user and generating one or more report metrics, wherein the data originates from multiple reader devices and multiple sensor control devices. In accordance with the disclosed subject matter, method 800 can be implemented to merge analyte data in order to generate different types of report metrics utilized in various reports. At Step 802, data is received from one or more reader devices 120 and combined for purposes of merging. At Step 804, the combined data is then de-duplicated to remove historical data from multiple readers originating from the same sensor control device. In accordance with the disclosed subject matter, the process of de-duplicating data can include (1) identifying or assigning a priority associated with each reader device from which analyte data is received, and (2) in the case where there is “duplicate” data, preserving the data associated with the reader device with a higher priority. In some embodiments, for example, a newer reader device (e.g., newer model, having a more recent version of software installed) is assigned a higher priority than an older reader device (e.g., older model, having an older version of software installed). In some embodiments, priority can be assigned by device type (e.g., smart phone having a higher priority over a dedicated reader).

Referring still to FIG. 8A, at Step 806, a determination is made as to whether one or more of the report metrics to be generated requires resolution of overlapping data. If not, at Step 808, a first type of report metric can be generated based on de-duplicated data without further processing. In some embodiments, for example, the first type of report metric can include average glucose levels used in reports, such as a snapshot or monthly summary report (as described with respect to FIGS. 5C and 5D). If it is determined that one or more of the report metrics to be generated requires resolution of overlapping data, then at Step 810, a method for resolving overlapping regions of data is performed. An example embodiment method for resolving overlapping regions of data is described below with respect to FIG. 8B. Subsequently, at Step 812, a second type of report metric based on data that has been de-duplicated and processed to resolve overlapping data segments, is generated. In some embodiments, for example, the second type of report metric can include low glucose event calculations used in reports, such as the daily log report (as described with respect to FIG. 5F).

FIG. 8B is a flow diagram depicting an example embodiment of a method 815 for resolving overlapping regions of analyte data, which can be implemented, for example, in Step 810 of method 800, as described with respect to FIG. 8A. At Step 817, the de-duplicated data from each reader (resulting from Step 804 of method 800, as described with respect to FIG. 8A) can be sorted from earliest to most recent. At Step 819, based on the report metric to be generated, the de-duplicated and sorted data is then isolated according to a predetermined period of time. In some embodiments, for example, if the report metric is a graph reflecting glucose values over a specific day, then the de-duplicated and sorted data can be isolated for that specific day. Next, at Step 821, contiguous sections of the de-duplicated and sorted data for each reader device are isolated. In accordance with the disclosed subject matter, non-contiguous data points can be discarded or disregarded (e.g., not used) for purposes of generating report metrics. At Step 823, for each contiguous section of de-duplicated and sorted data of a reader device, a determination is made as to whether there are any overlapping regions with other contiguous sections of de-duplicated and sorted data from other reader devices. At Step 825, for each overlapping region identified, the de-duplicated and sorted data from the reader device with the higher priority is preserved. At Step 827, if it is determined that all contiguous sections have been analyzed according to the previous steps, then method 815 ends at Step 829. Otherwise, method 815 then returns to Step 823 to continue identifying and resolving any overlapping regions between contiguous sections of de-duplicated and sorted data for different reader devices.

FIGS. 8C to 8E are graphs (840, 850, 860) depicting various stages of de-duplicated and sorted data from multiple reader devices, as the data is processed according to method 815 for resolving overlapping regions of data. Referring first to FIG. 8C, graph 840 depicts de-duplicated and sorted data from three different reader devices: a first reader 841 (as reflected by the circular data points), a second reader 842 (as reflected by diamond-shaped data points), and a third reader 843 (as reflected by the square-shaped data points). According to one aspect of graph 840, the data is depicted at Step 821 of method 815, after it has been de-duplicated, sorted, and isolated to a predetermined time period. As can be seen in FIG. 8C, a contiguous section of data for each of the three reader devices (841, 842, and 843) has been identified, and three traces are shown. According to another aspect of the graph 840, non-contiguous points 844 are not included in the three traces.

Referring next to FIG. 8D, graph 850 depicts the data from readers 841, 842, 843 at Step 823 of method 815, wherein three overlapping regions between the contiguous sections of data have been identified: a first overlapping region 851 between all three contiguous sections of data; a second overlapping region 852 between two contiguous sections of data (from reader device 842 and reader device 843); and a third overlapping region 853 between two contiguous sections of data (also from reader device 842 and reader device 843).

FIG. 8E is a graph 860 depicting data at Step 825 of method 815, wherein a single trace 861 indicates the merged, de-duplicated, and sorted data from three reader devices 841, 842, 843 after overlapping regions 851, 852, and 853 have been resolved by using the priority of each reader device. According to graph 860, the order of priority from highest to lowest is: reader device 843, reader device 842, and reader device 841.

Although FIGS. 8C, 8D, and 8E depict three contiguous sections of data with three discrete overlapping regions identified, those of skill in the art will understand that either fewer or more contiguous sections of data (and non-contiguous data points) and overlapping regions are possible. For example, those of skill in the art will recognize that where a user has only two reader devices, there may be fewer contiguous sections of data and overlapping regions, if any at all. Conversely, if a user has five reader devices, those of skill in the art will understand that there may be five contiguous sections of data with three or more overlapping regions.

### Example Embodiments of Sensor Transitioning

Example embodiments of methods for sensor transitioning will now be described. In accordance with the disclosed subject matter, as mobile computing and wearable technologies continue to advance at a rapid pace and become more ubiquitous, users are more likely to replace or upgrade their smart phones more frequently. In the context of analyte monitoring systems, it would therefore be beneficial to have sensor transitioning methods to allow a user to continue using a previously activated sensor control device with a new smart phone. In addition, it would also be beneficial to ensure that historical analyte data from the sensor control device could be backfilled to the new smart phone (and subsequently uploaded to the trusted computer system) in a user-friendly and secure manner.

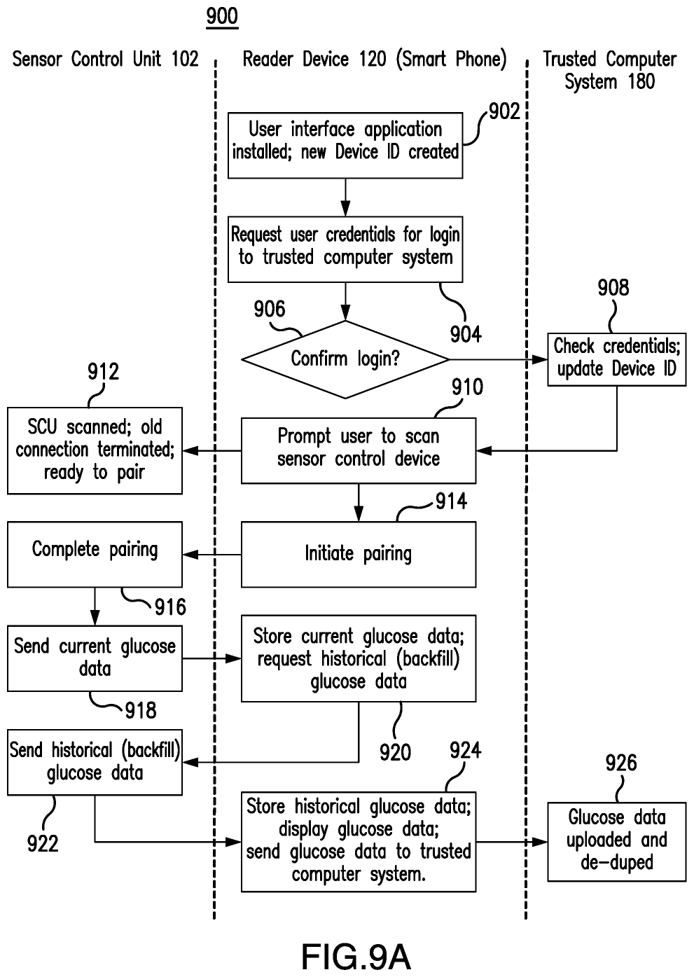

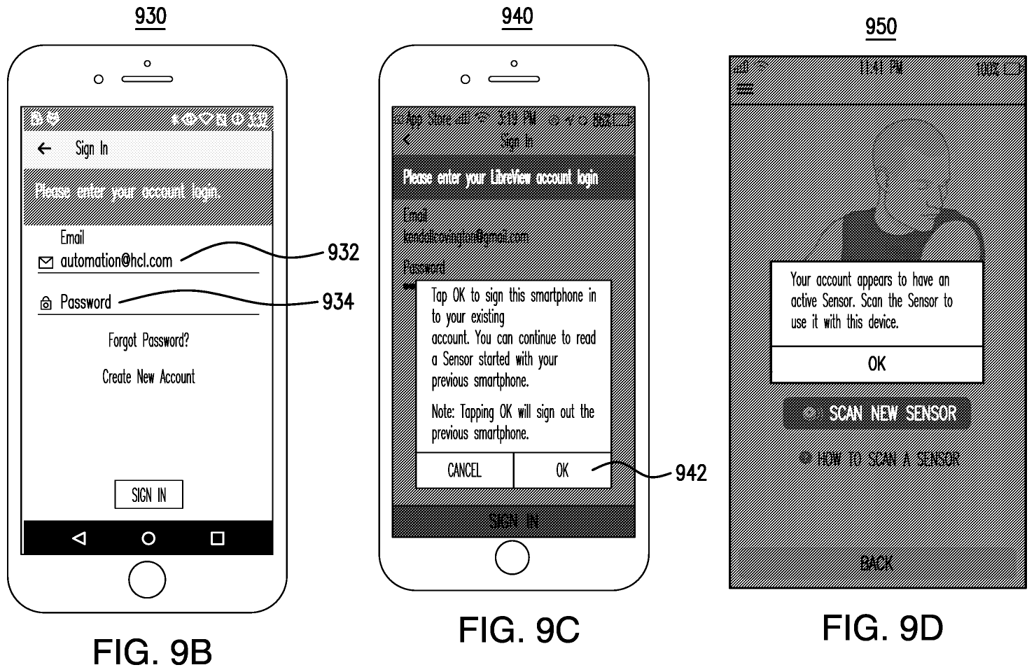

FIG. 9A is a flow diagram depicting an example embodiment of a method 900 for transitioning a sensor control device. In accordance with the disclosed subject matter, method 900 can be implemented in an analyte monitoring system to allow a user to continue using a previously activated sensor control device with a new reader device (e.g., smart phone). At Step 902, a user interface application (e.g., mobile software application or app) is installed on reader device 120 (e.g., smart phone), which causes a new unique device identifier, or “device ID,” to be created and stored on reader device 120. At Step 904, after installing and launching the app, the user is prompted to enter their user credentials for purposes of logging into trusted computer system 180 (e.g., cloud-based platform or server). An example embodiment of a GUI 930 for prompting the user to enter their user credentials is shown in FIG. 9B. According to an aspect of the embodiments, GUI 930 can include a username field 932, which can comprise a unique username or an e-mail address, and a masked or unmasked password field 934, to allow the user to enter their password.

Referring again to FIG. 9A, at Step 906, after user credentials are entered into the app, a prompt is displayed requesting user confirmation to login to trusted computer system 180. An example embodiment of GUI 940 for requesting user confirmation to login to trusted computer system 180 is shown in FIG. 9D. According to an aspect of the embodiments, GUI 940 can also include a warning, such as the one shown in FIG. 9D, that confirming the login will cause the user to be logged off from other reader devices (e.g., the user's old smart phone).

If the user confirms login, then at Step 908, the user's credentials are sent to trusted computer system 180 and subsequently verified. In addition, according to some embodiments, the device ID can also be transmitted from the reader device 120 to trusted computer system 180 and stored in a non-transitory memory of trusted computer system 180. According to some embodiments, for example, in response to receiving the device ID, trusted computer system 180 can update a device ID field associated with the user's record in a database.

After the user credentials are verified by trusted computer system 180, at Step 910, the user is prompted by the app to scan the already-activated sensor control device 102. In accordance with the disclosed subject matter, the scan can comprise bringing the reader device 120 in close proximity to sensor control device 102, and causing the reader device 120 to transmit one or more wireless interrogation signals according to a first wireless communication protocol. In some embodiments, for example, the first wireless communication protocol can be a Near Field Communication (NFC) wireless communication protocol. Those of skill in the art, however, will recognize that other wireless communication protocols can be implemented (e.g., infrared, UHF, 802.11x, etc.). An example embodiment of GUI 950 for prompting the user to scan the already-activated sensor control device 102 is shown in FIG. 9D.

Referring still to FIG. 9A, at Step 912, scanning of sensor control device 102 by reader device 120 causes sensor control device 102 to terminate an existing wireless communication link with the user's previous reader device, if there is currently one established. According to an aspect of the embodiments, the existing wireless communication link can comprise a link established according to a second wireless communication protocol that is different from the first wireless communication protocol. In some embodiments, for example, the second wireless communication protocol can be a Bluetooth or Bluetooth Low Energy protocol. Subsequently, sensor control device 102 enters into a “ready to pair” state, in which sensor control device 102 is available to establish a wireless communication link with reader device 120 according to the second wireless communication protocol.

At Step 914, reader device 120 initiates a pairing sequence via the second wireless communication protocol (e.g., Bluetooth or Bluetooth Low Energy) with sensor control device 102. Subsequently, at Step 916, sensor control device 102 completes the pairing sequence with reader device 120. At Step 918, sensor control device 102 can begin sending current glucose data to reader device 120 according to the second wireless communication protocol. In some embodiments, for example, current glucose data can be wirelessly transmitted to reader device 120 at a predetermined interval (e.g., every minute, every two minutes, every five minutes).

Referring still to FIG. 9A, at Step 920, reader device 120 receives and stores current glucose data received from sensor control device 102 in a non-transitory memory of reader device 120. In addition, according to some embodiments, reader device 120 can request historical glucose data from sensor control device 102 for backfilling purposes. According to some embodiments, for example, reader device 120 can request historical glucose data from sensor control device 102 for the full wear duration, which is stored in a non-transitory memory of sensor control device 102. In other embodiments, reader device 120 can request historical glucose data for a specific predetermined time range (e.g., from day 3 to present, from day 5 to present, last 3 days, last 5 days, lifecount >0, etc.). Those of skill will appreciate that other backfilling schemes can be implemented (such as those described with respect to FIGS. 6A and 6B), and are fully within the scope of this disclosure.

Upon receipt of the request at Step 922, sensor control device 102 can retrieve historical glucose data from a non-transitory memory and transmit it to reader device 120. In turn, at Step 924, reader device 120 can store the received historical glucose data in a non-transitory memory. In addition, according to some embodiments, reader device 120 can also display the current and/or historical glucose data in the app (e.g., on a sensor results screen). In this regard, a new reader can display all available analyte data for the full wear duration of a sensor control device. In some embodiments, reader device 120 can also transmit the current and/or historical glucose data to trusted computer system 180. At Step 926, the received glucose data can be stored in a non-transitory memory (e.g., a database) of trusted computer system 180.

In some embodiments, the received glucose data can also be de-duplicated prior to storage in non-transitory memory.

### Example Embodiments of Check Sensor and Replace Sensor System Alarms

Example embodiments of autonomous check sensor and replace sensor system alarms, and methods relating thereto, will now be described. In accordance with the disclosed subject matter, certain adverse conditions affecting the operation of the analyte sensor and sensor electronics can be detectable by the sensor control device. For example, an improperly inserted analyte sensor can be detected if an average glucose level measurement over a predetermined period of time is determined to be below an insertion failure threshold. Due to its small form factor and a limited power capacity, however, the sensor control device may not have sufficient alarming capabilities. As such, it would be advantageous for the sensor control device to transmit indications of adverse conditions to another device, such as a reader device (e.g., smart phone), to alert the user of those conditions.

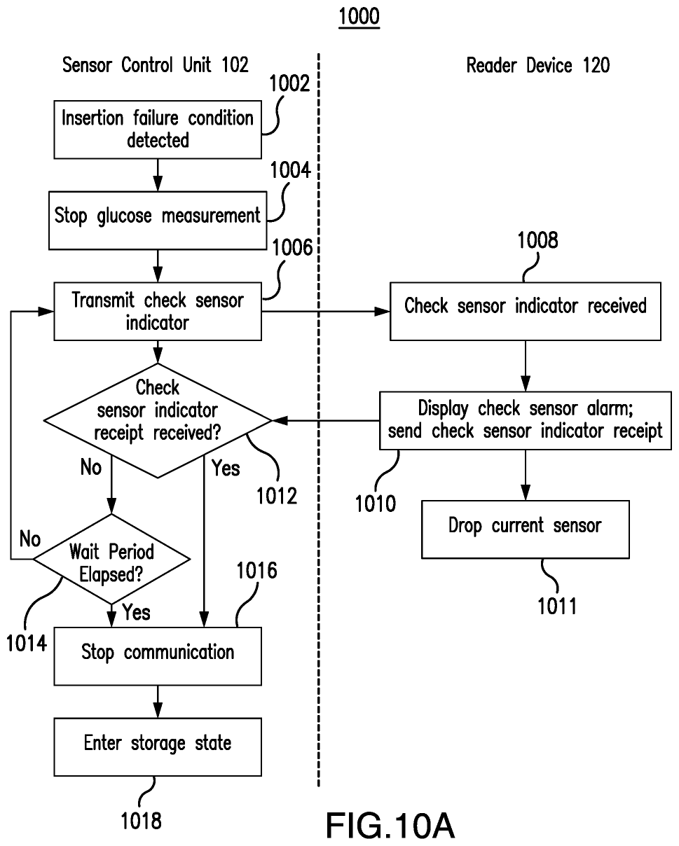

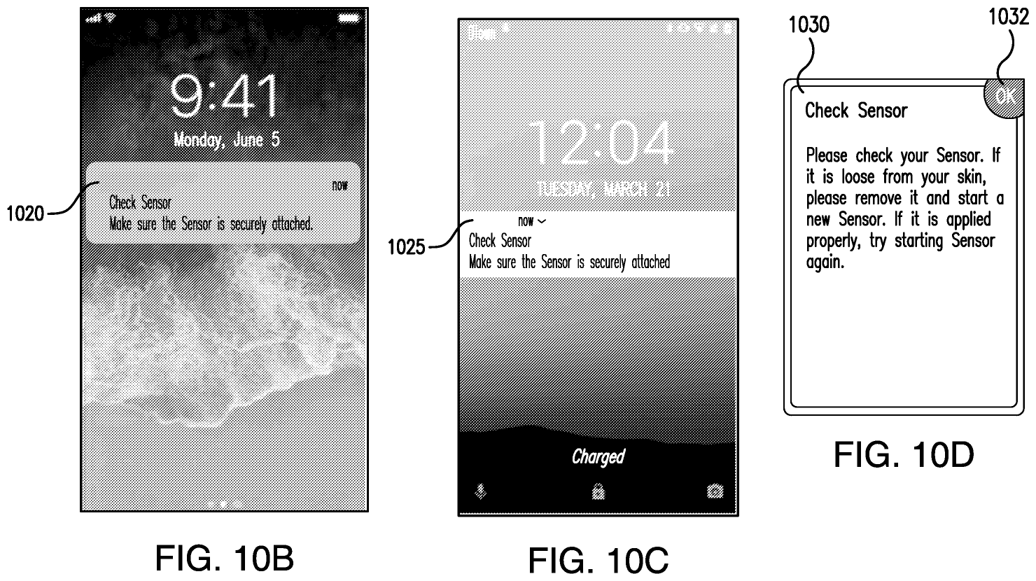

FIG. 10A is a flow diagram depicting an example embodiment of a method 1000 for generating a sensor insertion failure system alarm (also referred to as a “check sensor” system alarm). At Step 1002, a sensor insertion failure condition is detected by sensor control device 102. In some embodiments, for example, a sensor insertion failure condition can be detected when an average glucose value during a predetermined time period (e.g., average glucose value over five minutes, eight minutes, 15 minutes, etc.) is below an insertion failure glucose level threshold. At Step 1004, in response to the detection of the insertion failure condition, sensor control device 102 stops taking glucose measurements. At Step 1006, sensor control device 102 generates a check sensor indicator and transmits it via wireless communication circuitry to reader device 120. Subsequently, as shown at Steps 1012 and 1014, sensor control device 102 will continue to transmit the check sensor indicator until either: (1) a receipt of the indicator is received from reader device 120 (step 1012); or (2) a predetermined waiting period has elapsed (Step 1014), whichever occurs first.

According to another aspect of the embodiments, if a wireless communication link is established between sensor control device 102 and reader device 120, then reader device 120 will receive the check sensor indicator at Step 1008. In response to receiving the check sensor indicator, reader device 120 will display a check sensor system alarm at Step 1010. FIGS. 10B to 10D are example embodiments of check sensor system alarm interfaces, as displayed on reader device 120. In some embodiments, for example, the check sensor system alarm can be a notification box, banner, or pop-up window that is output to a display of a smart phone, such as interfaces 1020 and 1025 of FIGS. 10B and 10C. In some embodiments, the check sensor alarm can be output to a display on a reader device 120, such as a glucose meter or a receiver device, such as interface 1030 of FIG. 10D. According to the embodiments, reader device 120 can also transmit a check sensor indicator receipt back to sensor control device 102. In some embodiments, for example, the check sensor indicator receipt can be automatically generated and sent upon successful display of the check sensor system alarm 1020, 1025, or 1030. In other embodiments, the check sensor indicator receipt is generated and/or transmitted in response to a predetermined user input (e.g., dismissing the check sensor system alarm, pressing a confirmation ‘OK’ button 1032, etc.).

Subsequently, at Step 1011, reader device 120 drops sensor control device 102. In accordance with the disclosed subject matter, for example, Step 1011 can comprise one or more of: terminating an existing wireless communication link with sensor control device 102; unpairing from sensor control device 102; revoking an authorization or digital certificate associated with sensor control device 102; creating or modifying a record stored on reader device 120 to indicate that sensor control device 102 is in a storage state; or transmitting an update to trusted computer system 180 to indicate that sensor control device 102 is in a storage state.

Referring back to FIG. 10A, if either the check sensor indicator receipt is received (at Step 1012) by sensor control device 102 or the predetermined wait period has elapsed (Step 1014), then at Step 1016, sensor control device 102 stops the transmission of check sensor indicators. Subsequently, at Step 1018, sensor control device 102 enters a storage state in which sensor control device 102 does not take glucose measurements and the wireless communication circuitry is either de-activated or transitioned into a dormant mode. According to one aspect, while in a ‘storage state,’ sensor control device 102 can be re-activated by reader device 120.

Although method 1000 of FIG. 10A is described with respect to glucose measurements, those of skill in the art will appreciate that sensor control device 102 can be configured to measure other analytes (e.g., lactate, ketone, etc.) as well. In addition, although method 1000 of FIG. 10A describes certain method steps performed by reader device 120 (e.g., receiving check sensor indicator, displaying a check sensor system alarm, and sending a check sensor indicator receipt), those of skill in the art will understand that any or all of these method steps can be performed by other devices in an analyte monitoring system, such as, for example, a local computer system, a wearable computing device, or a medication delivery device. It will also be understood by those of skill in the art that method 1000 of FIG. 10A can combined with any of the other methods described herein, including but not limited to method 700 of FIG. 7, relating to expired and or failed sensor transmissions.

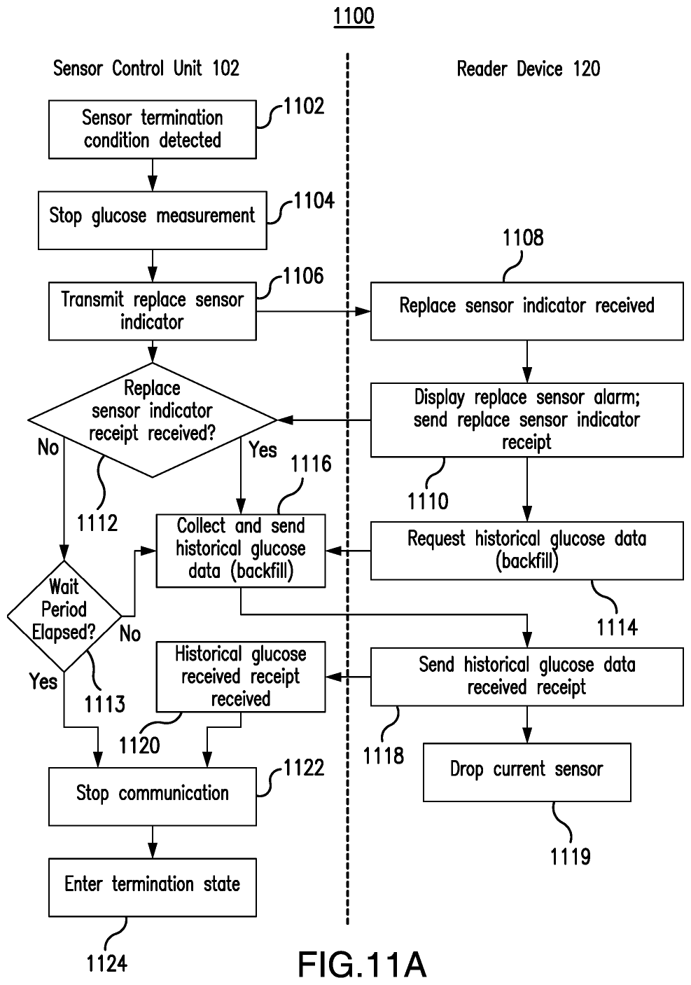

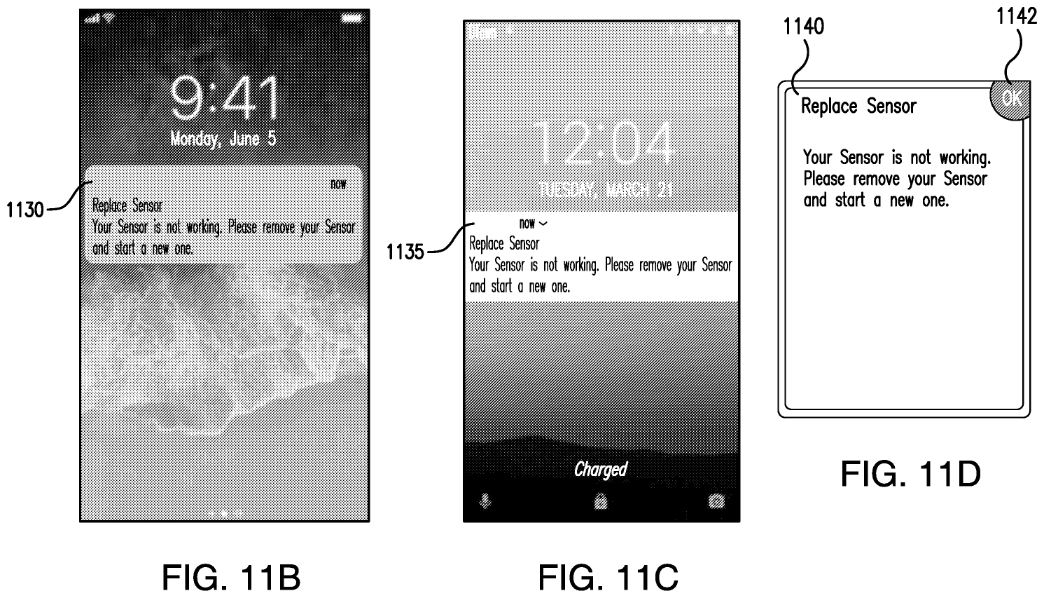

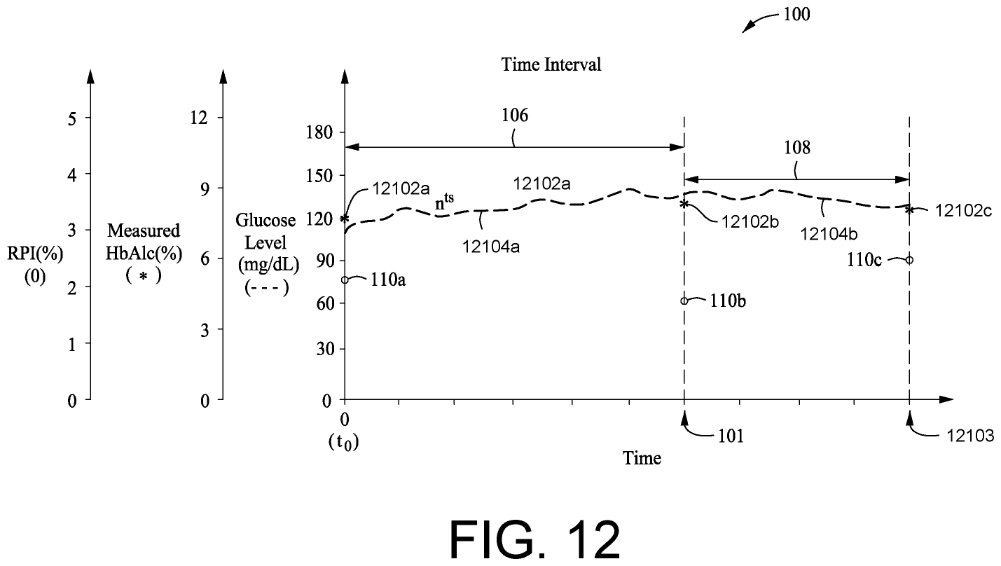

FIG. 11A is a flow diagram depicting an example embodiment of a method 1100 for generating a sensor termination system alarm (also referred to as a “replace sensor” system alarm). At Step 1102, a sensor termination condition is detected by sensor control device 102. As described earlier, a sensor termination condition can include, but is not limited to, one or more of the following: a FIFO overflow condition detected, a sensor signal below a predetermined insertion failure threshold, moisture ingress detected, an electrode voltage exceeding a predetermined diagnostic voltage threshold, an early signal attenuation (ESA) condition, or a late signal attenuation (LSA) condition, to name a few.

At Step 1104, in response to the detection of a sensor termination condition, sensor control device 102 stops taking glucose measurements. At Step 1106, sensor control device 102 generates a replace sensor indicator and transmits it via wireless communication circuitry to reader device 120. Subsequently, at Step 1112, sensor control device 102 will continue to transmit the replace sensor indicator while determining whether a replace sensor indicator receipt has been received from reader device 102. In accordance with the disclosed subject matter, sensor control device 102 can continue to transmit the replace sensor indicator until either: (1) a predetermined waiting period has elapsed (Step 1113), or (2) a receipt of the replace sensor indicator is received (Step 1112) and sensor control device 102 has successfully transmitted backfill data (Steps 1116, 1120) to reader device 120.

Referring still to FIG. 11A, if a wireless communication link is established between sensor control device 102 and reader device 120, then reader device 120 will receive the replace sensor indicator at Step 1108. In response to receiving the replace sensor indicator, reader device 120 will display a replace sensor system alarm at Step 1110. FIGS. 11B to 11D are example embodiments of replace sensor system alarm interfaces, as displayed on reader device 120. In some embodiments, for example, the replace sensor system alarm can be a notification box, banner, or pop-up window that is output to a display of a smart phone, such as interfaces 1130 and 1135 of FIGS. 11B and 11C. In some embodiments, the check sensor alarm can be output to a display on a reader device 120, such as a glucose meter or a receiver device, such as interface 1140 of FIG. 11D. According to the embodiments, to acknowledge receipt of the indicator, reader device 120 can also transmit a replace sensor indicator receipt back to sensor control device 102. In some embodiments, for example, the replace sensor indicator receipt can be automatically generated and sent upon successful display of the replace sensor system alarm 1130, 1135, or 1140. In other embodiments, the replace sensor indicator is generated and/or transmitted in response to a predetermined user input (e.g., dismissing the check sensor system alarm, pressing a confirmation ‘OK’ button 1142, etc.).

At Step 1114, after displaying the replace sensor system alarm and transmitting the replace sensor indicator receipt, reader device 120 can then request historical glucose data from sensor control device 102. At Step 1116, sensor control device 102 can collect and send to reader device 120 the requested historical glucose data. In accordance with the disclosed subject matter, the step of requesting, collecting, and communicating historical glucose data can comprise a data backfilling routine, such as the methods described with respect to FIGS. 6A and 6B.

Referring again to FIG. 11A, in response to receiving the requested historical glucose data, reader device 120 can send a historical glucose data received receipt to sensor control device 102 at Step 1118. Subsequently, at Step 1119, reader device 120 drops sensor control device 102. In accordance with the disclosed subject matter, for example, Step 1119 can comprise one or more of: terminating an existing wireless communication link with sensor control device 102; unpairing from sensor control device 102; revoking an authorization or digital certificate associated with sensor control device 102; creating or modifying a record stored on reader device 120 to indicate that sensor control device 102 has been terminated; or transmitting an update to trusted computer system 180 to indicate that sensor control device 102 has been terminated.

At Step 1120, sensor control device 102 receives the historical glucose data received receipt. Subsequently, at Step 1122, sensor control device 102 stops the transmission of the replace sensor indicator and, at Step 1124, sensor control device 102 can enter into a termination state in which sensor control device 102 does not take glucose measurements and the wireless communication circuitry is either de-activated or in a dormant mode. In accordance with the disclosed subject matter, when in a termination state, sensor control device 102 cannot be re-activated by reader device 120.

Although method 1100 of FIG. 11A is described with respect to glucose measurements, those of skill in the art will appreciate that sensor control device 102 can be configured to measure other analytes (e.g., lactate, ketone, etc.) as well. In addition, although method 1100 of FIG. 11A describes certain method steps performed by reader device 120 (e.g., receiving replace sensor indicator, displaying a replace sensor system alarm, and sending a replace sensor indicator receipt), those of skill in the art will understand that any or all of these method steps can be performed by other devices in an analyte monitoring system, such as, for example, a local computer system, a wearable computing device, or a medication delivery device. It will also be understood by those of skill in the art that method 1100 of FIG. 11A can combined with any of the other methods described herein, including but not limited to method 700 of FIG. 7, relating to expired and or failed sensor transmissions.

### Example Embodiments of Reports Comprising a Plurality of Interfaces

Example embodiments of reports comprising a plurality of interfaces will now be described. In accordance with the disclosed subject matter, a report including a plurality of the interfaces disclosed herein may be presented to a user. In accordance with the disclosed subject matter, the interfaces can include any combination of measured interfaces based on current or measured analyte values, physiological parameter interfaces based on the physiological parameters disclosed herein, and personalized interfaces based on personalized glucose metrics disclosed herein.

In view of the above and in accordance with the disclosed subject matter, a glucose monitoring system is provided comprising a sensor control device, comprising an analyte sensor coupled with sensor electronics and configured to transmit data indicative of an analyte level of a subject, and a reader device. The reader device of the disclosed subject matter comprises a wireless communication circuitry configured to receive the data indicative of the analyte level and a glycated hemoglobin level for the subject, a non-transitory memory, and at least one processor. The processor is communicatively coupled to the non-transitory memory and the analyte sensor and configured to calculate a plurality of personalized glucose metrics for the subject using at least one physiological parameter and at least one of the received data indicative of the analyte level or the received glycated hemoglobin level, and display, on a display of the reader device, a report comprising a plurality of interfaces including at least two or more of the received data indicative of the analyte level, the received glycated hemoglobin level, or the calculated plurality of personalized glucose metrics, wherein the plurality of interfaces comprising the report are based on a user type. According to embodiments, the at least one physiological parameter is selected from the group consisting of: a red blood cell glucose uptake, a red blood cell lifespan, a red blood cell glycation rate constant, a red blood cell generation rate constant, a red blood cell elimination constant, and an apparent glycation constant. For example, not limitation, in further embodiments, the plurality of interfaces includes the at least one physiological parameter for the subject.

According to embodiments, contents of a report may vary based on different user types (for example, not limitation, subjects, health care providers, caretakers, etc.). As embodied herein, the plurality of interfaces comprising the report are predetermined based on the user type or can be selected by the user. According to embodiment, the user type includes a health care professional. For example, without limitation, in a further embodiment, the plurality of interfaces includes a glucose monitoring data interface, a glycated hemoglobin interface, a personalized A1c interface, a personalized glucose interface, a personalized average glucose, and a personalized time in range interface. According to embodiment, the user type includes the subject. For example, without limitation, in a further embodiment, the plurality of interfaces a glucose monitoring data interface, a glycated hemoglobin interface, a mean glucose interface, and a time in range interface.

According to embodiments, subjects using the analyte monitoring systems can only view graphical interfaces displaying measured analyte measurements, or personalized analyte measurements, but not both. For example, it can be beneficial to minimize confusion by showing graphical interfaces with slightly different data (such as between measured and personalized). As embodied herein, the selection of which interfaces can be included in a report is dependent on whether the personalized glucose metrics have been approved or designated for research purposes or clinical purposes by the appropriate regulatory authority.

According to embodiments, personalized glucose metrics can include one or more of a personalized A1c or adjusted A1c, glucose-determined A1c or calculated A1c, personalized glucose, personalized average glucose, and personalized time in rage. According to embodiments, at least one processor is configured to calculate a plurality of personalized glucose targets corresponding to the calculated plurality of personalized glucose metrics. According to embodiments, the plurality of interfaces further includes the plurality of personalized glucose targets. According to embodiments, personalized glucose targets can include one or more of personalized glucose target range and personalized target average glucose. According to embodiments, personalized glucose target range can include a personalized lower glucose limit and/or a personalized upper glucose limit.

FIG. 24 shows an exemplary report 1400 including four different measured interfaces associated with exemplary subject J17: a glucose monitoring data interface 2401 which includes a graphical representation of measured glucose measurements from an analyte monitoring device over a predetermined period of time, HbA1c interface 2402 including a graphical representation of HbA1c measurements (shown as dots 1402a) over a predetermined period of time and a graphical representation of calculated A1c (“cA1c”) or glucose derived A1c, a mean glucose interface 1403 including a graphical representation of measured 14-day mean glucose (148 mg/dL) over a predetermined period of time, and time-in-range interface 1404 including a graphical representation of measured time in range metrics (75% over 180 mg/dL and 2% below 70 mg/dL, as shown) over a predetermined period of time. As embodied herein, HbA1c measurements can include laboratory A1c measurements. In further embodiment, for example, not limitation, the reader device wirelessly receives the glycated hemoglobin level for the subject from an electronic medical records system, cloud-based database, from the subject from a QR code, from the subject using a home test kit which can optionally be mailed to a laboratory for analysis. As embodied herein, FIG. 24 can include any of the interfaces disclosed herein.

As embodied herein, as shown in FIG. 24, glucose monitoring data interface 2401 can including a graphical representation (shown as dashed line) of target glucose range 2401b,c in the foreground. Target glucose range 2401b,c can include personalized target glucose range, as described herein. As embodied herein, as shown in FIG. 24, HbA1c interface 2402 can include a graphical representation (shown as solid line) of target HbA1c 2402b (for example, not limitation, 6.5%). As embodied herein, as shown in FIG. 24, mean glucose interface 1403 can include a graphical representation (shown as solid line) of target average glucose 1403a.

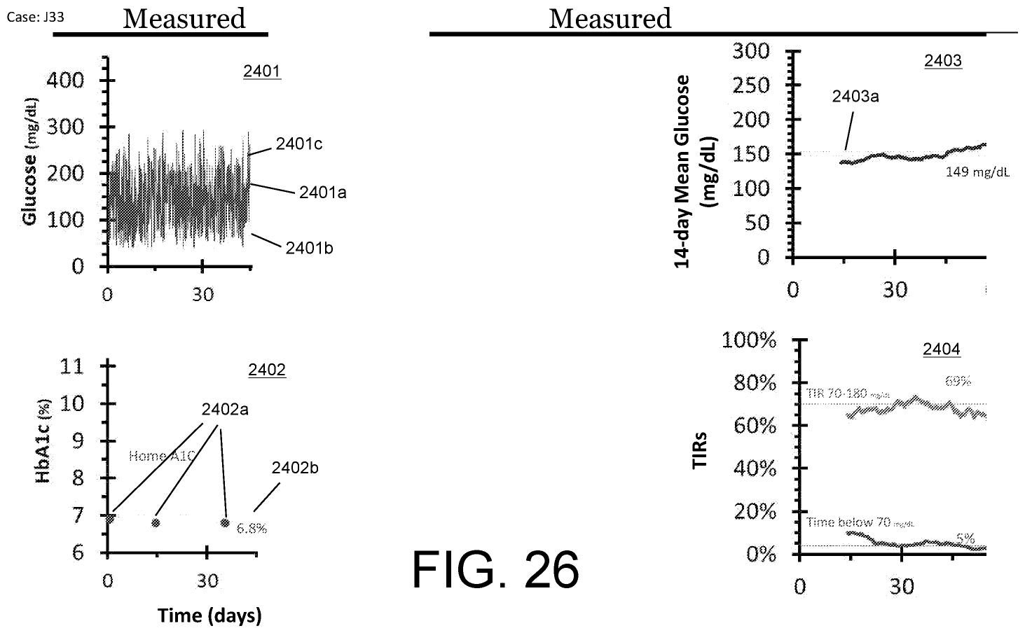

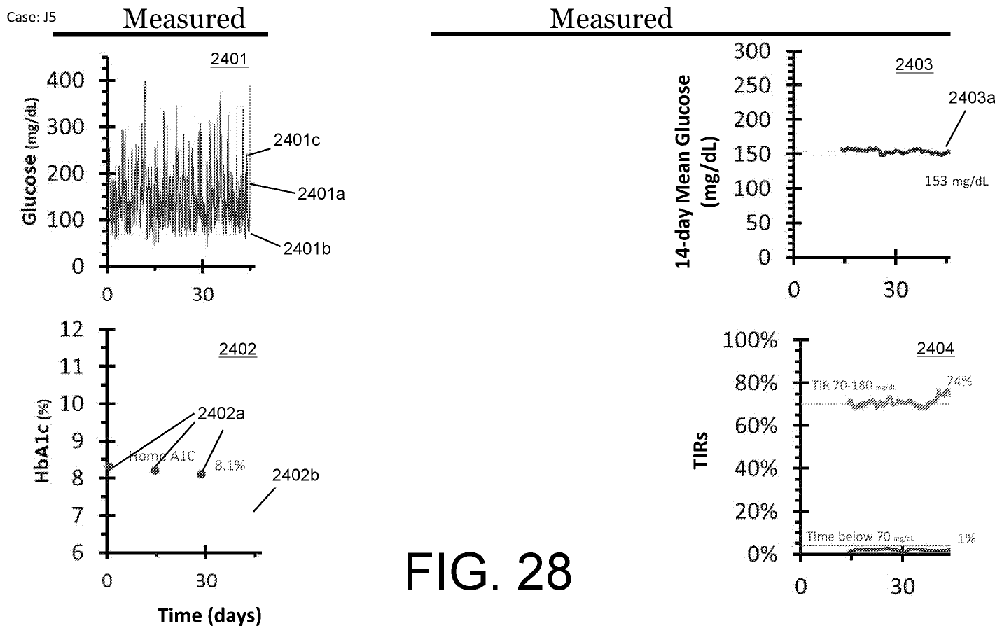

As embodied herein, as can be seen in FIG. 24, the predetermined time period can be 45 days. As embodied herein, the predetermined time period can be in five-minute increments, with a total of twelve hours of data. Those of skill in the art will appreciate, however, that other time increments (e.g., 30 days) and durations of analyte data can be utilized and are fully within the scope of this disclosure. FIGS. 26 and 28 similarly provide interfaces for exemplary subjects J33 and J5, respectively.

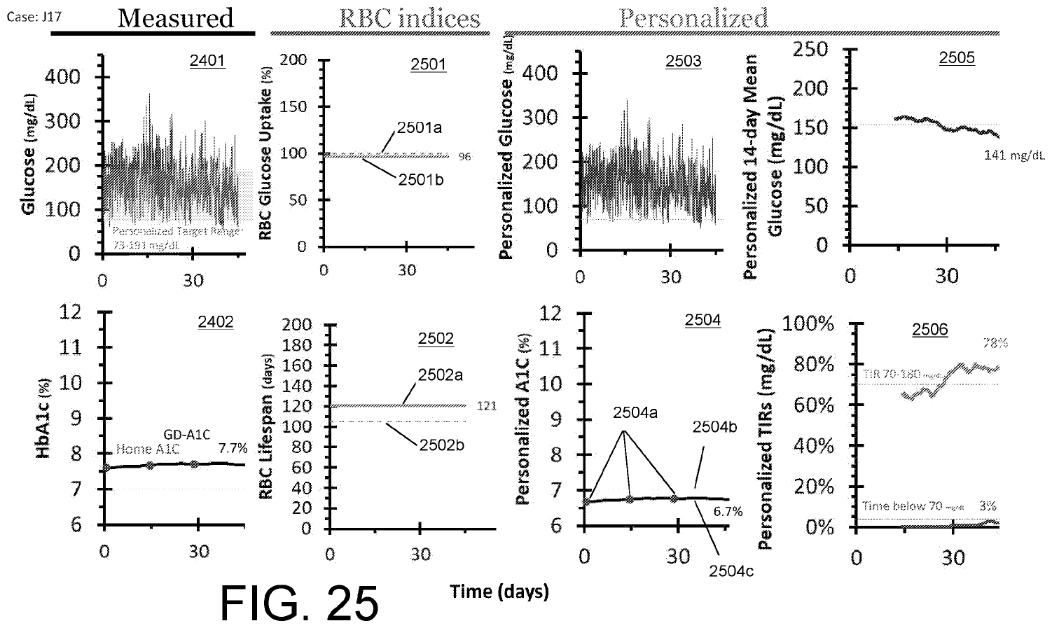

FIG. 25 shows an exemplary report 1500 including eight different interfaces associated with exemplary subject J17. As shown in FIG. 25, report 1500 can include measured interfaces, physiological parameter interfaces, and personalized interfaces. As embodied herein, FIG. 25 can include any of the interfaces disclosed herein.

According to embodiment disclosed herein, measured interfaces can include, for example, not limitation, a glucose monitoring data interface 2401 and HbA1c interface 2402, as shown in FIG. 25. As embodied herein, HbA1c interface 2402 can include a calculated HbA1c (cA1c or GD-A1c) curve fitted through the HbA1c measurements, as described herein and in WO2021/108419 and WO2020/086934 to Xu, which are incorporated by reference in its entirety herein.

According to embodiment disclosed herein, physiological parameter interfaces can include for example, not limitation, red blood cell glucose uptake interface 2501 and red blood cell lifespan interface 2502, as shown in FIG. 25. As embodied herein, red blood cell glucose uptake interface 2501 can include a graphical representation of the subject's red blood cell glucose uptake (solid line) 2501a and a reference red blood cell glucose uptake (dashed line) 2501b over a predetermined period of time. As embodied herein, red blood cell lifespan interface 2502 can include a graphical representation of the subject's red blood cell lifespan (solid line) 2502a and a reference red blood cell lifespan (dashed line) 2502b over a predetermined period of time. As can be seen in FIG. 23 and illustrated in FIG. 25, subject J17's red blood cell glucose uptake is 96% and red blood cell lifespan is 121 days. The subject's red blood cell glucose uptake and red blood cell lifespan can be calculated using the models, described herein and in WO2021/108419 and WO2020/086934 to Xu, which are incorporated by reference in its entirety herein. As embodied herein, physiological parameter interfaces can include any other physiological parameters as described herein and in WO2021/108419 and WO2020/086934 to Xu, which are incorporated by reference in its entirety herein.

According to embodiment disclosed herein, personalized interfaces can include for example, not limitation, personalized glucose interface 2503, personalized A1c interface 2504, personalized 14-day mean glucose interface 2505, and personalized time in ranges interface 2506, as shown in FIG. 25. As embodied herein, FIG. 25 can include any of the personalized interfaces disclosed herein. Personalized glucose interface 2503 can include a graphical representation of the subject's glucose monitoring data interface personalized using the models as described herein and in WO2021/108419 and WO2020/086934 to Xu, which are incorporated by reference in its entirety herein. As embodied herein, as shown in FIG. 25, personalized glucose interface 2503 can including target glucose range 2401b,c in the foreground. Target glucose range 2401b,c can include personalized target glucose range, as described herein.

According to embodiment disclosed herein, personalized A1c interface 2504 can include a graphical representation of the subject's adjusted or personalized A1c (shown as a dots 2504a) and adjusted cHbA1c (shown as curve fit 2504c), calculated using the models as described herein and in WO2021/108419 and WO2020/086934 to Xu, which are incorporated by reference in its entirety herein. As embodied herein, personalized A1c interface 2504 can include a graphical representation (shown as solid line) of target HbA1c 2504b (for example, not limitation, 6.5%).

According to embodiment disclosed herein, personalized 14-day mean glucose interface 2505 can include a mean glucose interface 1403 including a graphical representation of personalized 14-day mean glucose (141 mg/dL as shown) over a predetermined period of time. As embodied herein, as shown in FIG. 25, personalized mean glucose interface 2503 can include a graphical representation (shown as solid line) of target average glucose 1403a.

According to embodiment disclosed herein, personalized time in ranges interface 2506, can include a graphical representation of personalized time in range metrics (78% over 180 mg/dL and 3% below 70 mg/dL, as shown) over a predetermined period of time.

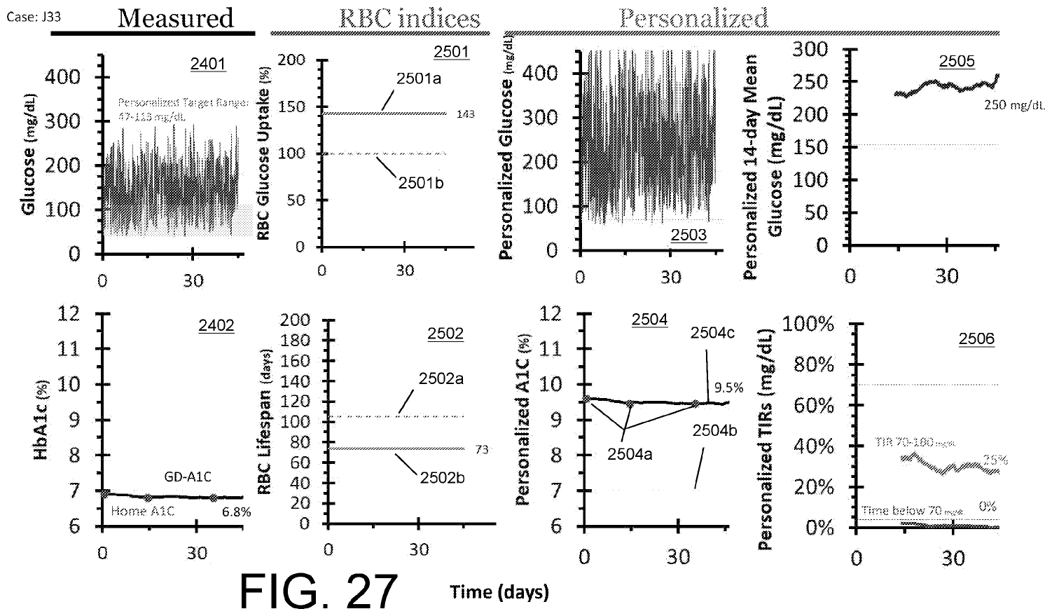

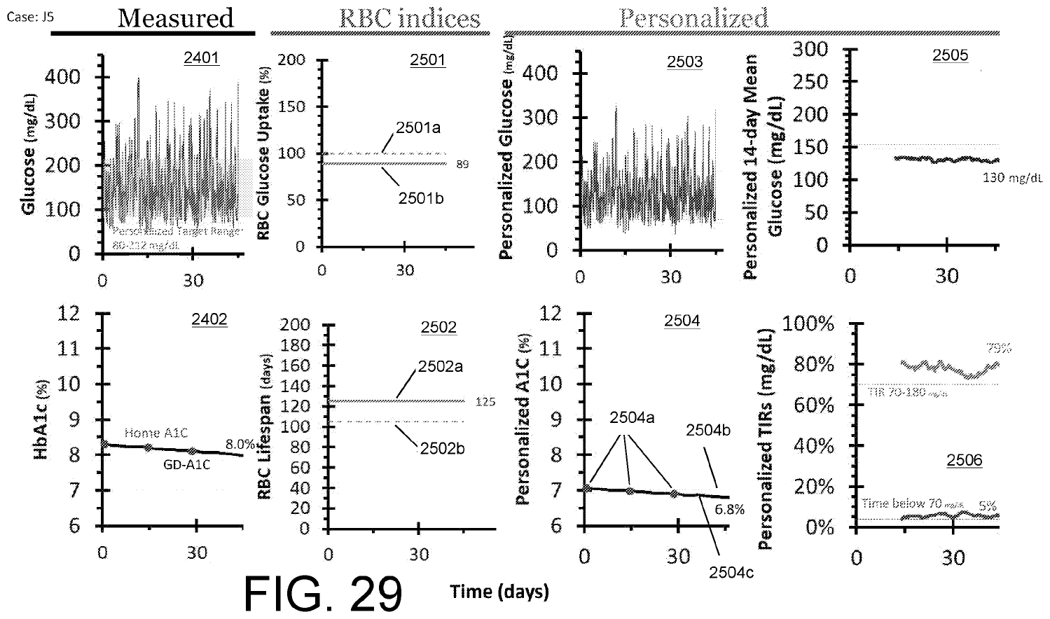

As embodied herein, as can be seen in FIG. 25, the predetermined time period can be 45 days. As embodied herein, the predetermined time period can be in five-minute increments, with a total of twelve hours of data. Those of skill in the art will appreciate, however, that other time increments, and durations of analyte data can be utilized and are fully within the scope of this disclosure. FIGS. 27 and 29 similarly provide graphical illustration of four different glucose metrics for J33 and J5, respectively.

According to embodiments disclosed herein, reports 1400 or 1500 can include a variety of measured interfaces, physiological parameter interfaces, or personalized interfaces based on user type. For example, health care providers (HCPs) and caretakers may benefit from seeing a comparison of measured interfaces and personalized interfaces, for example, to assess how much the two differ and to assess diagnosis and treatment options accordingly. As such, in an embodiment, contents of a report for an HCP can include a predetermined set of measured interfaces, physiological parameter interfaces, and personalized interfaces, for example, not limitation, as shown in report 1500. According to embodiments, HCPs can have the greatest access to information, including measured analyte measurements, personalized analyte measurement, and physiological parameters (for example, not limitation, RBC glucose uptake and RBC lifespan as shown in FIGS. 25, 27, 29, or as a second report as discussed above) as determined using models described herein. As embodied herein, in an embodiment, contents of a report for the subject can include a predetermined set of measured interfaces, physiological parameter interfaces, and/or personalized interfaces. For example, not limitation, a report generated for a user can include measured interfaces, as shown in report 1400. As embodied herein, a user type can include, for example, not limitation, the subject, a health care provider, a caretaker, an insurance provider, etc.

As embodied herein, a user (e.g., the subject, a HCP, a caretaker, an insurance provider, etc.) may select which interfaces comprise the report. For example, not limitation, the user may choose any combination of measured interfaces, personalized interfaces, and physiological parameter interface disclosed herein.

According to an embodiment, a user can select whether to view a sensor result interface as disclosed herein displaying measured analyte measurement (for example, not limitation, such as those shown in FIGS. 24, 26, and 28) over a predetermined period of time, or personalized measurements (for example, not limitation, such as those shown in FIGS. 25, 27, and 29) over the same predetermined period of time, or both. As embodied herein, the user can toggle or switch between viewing a sensor result interface with measured analyte measurements over a predetermined period of time and viewing the same sensor interface with personalized analyte measurements over the same predetermined period of time. For example, not limitation, a user can switch between a mean glucose interface 1403 including a graphical representation of average glucose level over 45 a day (for example, not limitation, such as that shown in FIG. 24) and a personalized mean glucose interface 2505 including a graphical representation of personalized average glucose level over the same predetermined period of time (for example, not limitation, such as that shown in FIG. 25). According to embodiments, a user can similarly switch between any of the other measured interfaces shown in FIGS. 24, 26, 28 and personalized interfaces shown in FIG. 25, 27, 29 (for example, without limitation, A1c interface, glucose interface, 14-day mean glucose interface, and time in range interface, etc.). Those of skill in the art will appreciate, however, that other time increments and durations of analyte data can be utilized and are fully within the scope of this disclosure. According to embodiments, the sensor results interfaces, analyte level and trend alert interfaces, time in range interfaces, and/or sensor usage interfaces as described herein can similarly be selected by a user to display measured analyte measurements over a predetermined period of time, and/or personalized analyte measurements over a predetermined period of time.

According to embodiments, the combined data can be used in conjunction with any of the graphical user interfaces described above. According to embodiments of the present disclosure, a user (e.g., a user, health care provider, caretaker, etc.) can personalize any of the graphical interfaces described above. Furthermore, an Ambulatory Glucose Profile Report (“AGP Report”) (for example, not limitation, such as the one proposed by the International Diabetes Center (“IDC”), which is incorporated by reference in its entirety and be found on the website, http://www.agpreport.org/agp/agpreports) can be modified to include any of the graphical interfaces or personalized metrics described herein. For example, not limitation, IDC's AGP Report Version 5 can be modified by replacing Glucose Management Indicator (GMI) with Personalized A1c. Furthermore, a graphical interface for reporting Personalized A1c can be achieved by combining any of the graphical components described herein. For example, in one embodiment, a graphical user interface 3000 can include at least the Time-in-Ranges GUI 340 as depicted in FIG. 3F, the glucose trend interface 517 as described herein, and the health information interface 518 as described herein. Interface 3000 can include the patient's name, date of birth (“DOB”), the time period which the report covers, and the time percentage of time in that time period that the continuous glucose monitor was active. As can be seen in FIG. 3, the time period can be 14 days. According to embodiments, time period can be any other period of time (for example, without limitation, 1 day, 2 days, 3, days, 7 days, 30 days, 45 days, etc. or any other period of time). According to embodiments the time period can be selected by the patient or the health care provider.

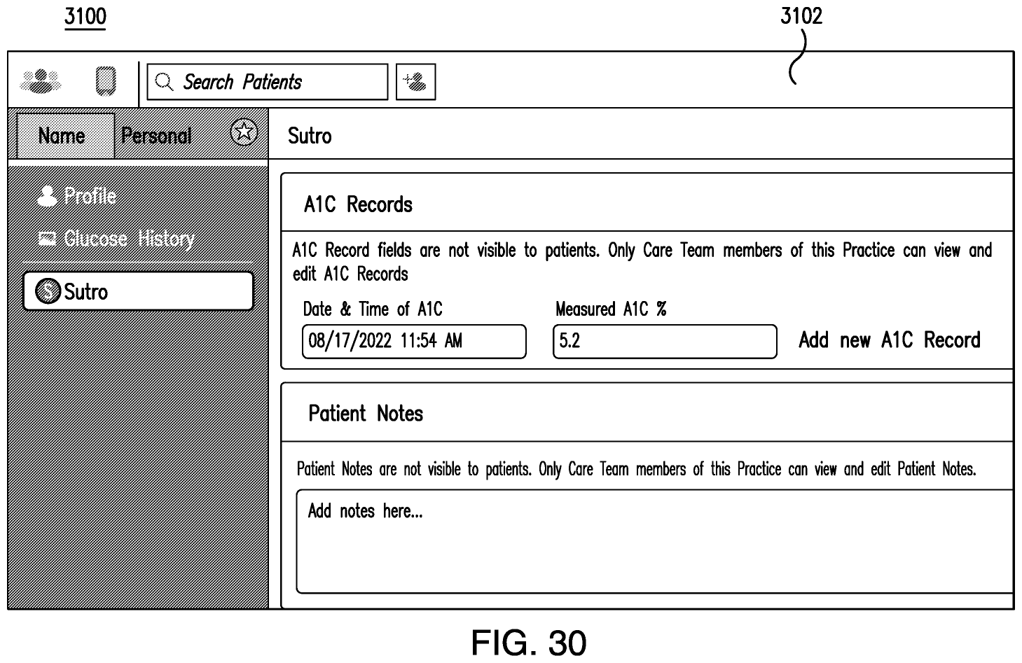

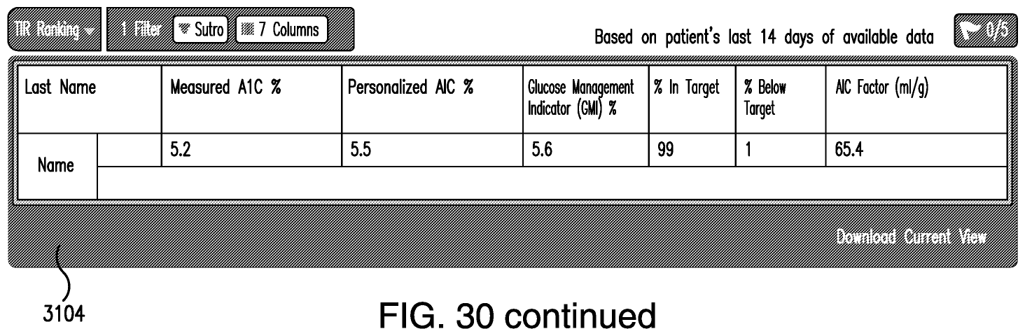

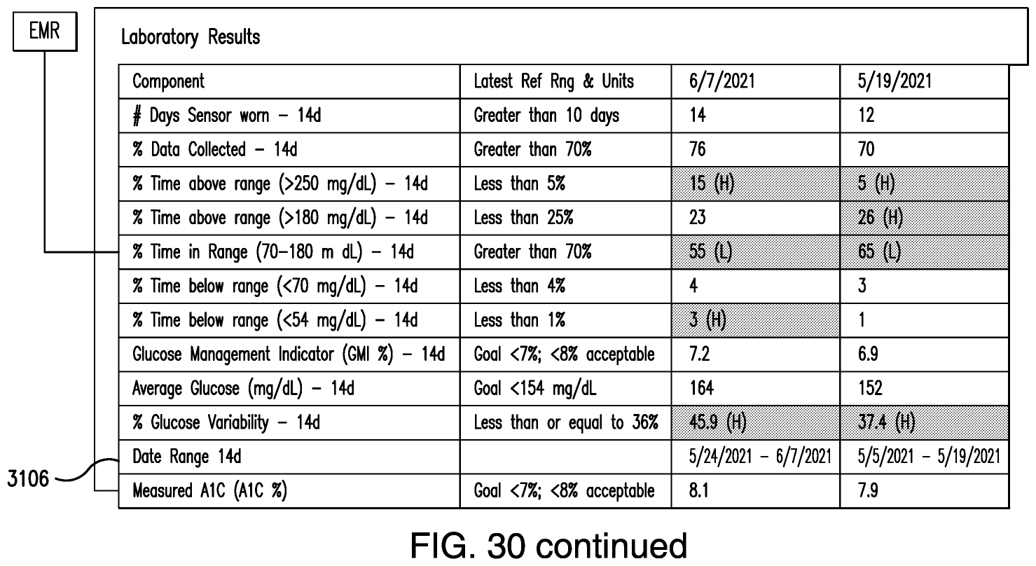

According to FIG. 30, another GUI can provide an interface for healthcare providers' use. For example, a provider interface 3100 can include an input interface 3102 for a provider to input A1c records, which can include a lab measured A1c value. Similarly, the provider interface 3100 can also include an output interface 3104 which can include a measured A1c and personalized A1c determined based on the measured A1c. The output interface 3104 can also include other data such as GMI, percent of time in target, percent of time below target, and personalized A1c factor (also known as an “adjusted glycation ratio” or “AGR” and as disclosed in U.S. patent application Ser. No. 18/052,805, which is incorporated by reference herein in its entirety). According to embodiments of the present disclosure, provider interface 3100 can also include a medical records interface 3106 for displaying electronic medical records (“EMR”). According to embodiments of the present disclosure, the EMR can include data such as time a sensor is worn, data collected, time in, above, or below range, measured A1c, personalized A1c, and more. According to embodiments, interface 3106 can include records over a period of time. For example, as can be seen in FIG. 30, interface 3106 can include records in a tabular format for each month data is collected and/or analyzed.

As disclosed in U.S. patent application Ser. Nos. 17/832,537 and 18/052,805, which are incorporated by reference in their entirety, HbA1c or HbA1c Target measurement can be adjusted by a user's Apparent Glycation Ration (“AGR”) (also referred to as “personalized A1c factor” or “personalized HbA1c factor”). For example, Table 3 shows an “adjusted” HbA1c target measurement based on AGR. More specifically, as can be seen in Table 3, an Ale target of 6.0 adjusted by AGR of 60 provides an adjusted A1c target is 5.5. Similarly, an A1c target of 6.0 adjusted by AGR of 65 provides an adjusted A1c target of 6.0. Alternatively, a measured A1c value can be similarly adjusted using the AGR to provide an adjusted Ale value (or a personalized Ale value). Presenting this information to subjects and health care providers can help them make more accurate and informed diabetes diagnosis and treatment based at least on the subject's individual demographic metrics and/or physiology.

TABLE 3

| | | Adjusted A1c target (%) based on AGR | | | | |
|---|---|---|---|---|---|---|
| A1C Target (%) | | 6.0 | 6.5 | 7.0 | 7.5 | 8.0 |
| AGR (ml/g) | 60 | 5.5 | 6.0 | 6.5 | 7.0 | 7.5 |
| | 65 | 6.0 | 6.5 | 7.0 | 7.5 | 8.0 |
| | 70 | 6.4 | 7.0 | 7.5 | 8.1 | 8.6 |
| | 75 | 6.8 | 7.4 | 8.0 | 8.6 | 9.2 |
| | 80 | 7.2 | 7.9 | 8.5 | 9.1 | 9.7 |

Thus, by measuring A1c, determining a personalized A1c factor, and applying the factor to the measured A1c, a personalized A1c can be determined.

While the disclosed subject matter is described herein in terms of certain illustrations and examples, those skilled in the art will recognize that various modifications and improvements may be made to the disclosed subject matter without departing from the scope thereof. Moreover, although individual features of one embodiment of the disclosed subject matter may be discussed herein or shown in the drawings of one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for the purposes of illustration and description. It is not intended to be exhaustive or to limit the disclosed subject matter to those embodiments disclosed.

The description herein merely illustrates the principles of the disclosed subject matter. Various modifications and alterations to the described embodiments will be apparent to those skilled in the art in view of the teachings herein. Accordingly, the disclosure herein is intended to be illustrative, but not limiting, of the scope of the disclosed subject matter.

What is claimed is:

1. A glucose monitoring system, comprising:
a sensor control device comprising an analyte sensor coupled with sensor electronics, the sensor control device configured to transmit data indicative of an analyte level of a subject; and
a reader device comprising:
a wireless communication circuitry configured to receive the data indicative of the analyte level and a glycated hemoglobin level for the subject;
a non-transitory memory;
at least one processor communicatively coupled to the non-transitory memory and the analyte sensor and configured to:
calculate a plurality of personalized glucose metrics for the subject using at least one physiological parameter and at least one of the received data indicative of the analyte level or the received glycated hemoglobin level; and
display, on a display of the reader device, a report comprising a plurality of interfaces including at least two or more of the received data indicative of the analyte level, the received glycated hemoglobin level, or the calculated plurality of personalized glucose metrics,
wherein the plurality of interfaces comprising the report are based on a user type.

2. The system of claim 1, wherein the plurality of personalized glucose metrics includes one or more of an adjusted A1c, a calculated A1c, an adjusted calculated A1c, a personalized glucose, a personalized average glucose, or a personalized time in range.

3. The system of claim 2, wherein the at least one processor is further configured to calculate a plurality of personalized glucose targets corresponding to the calculated plurality of personalized glucose metrics.

4. The system of claim 3, wherein the plurality of interfaces further includes the plurality of personalized glucose targets.

5. The system of claim 3, wherein the plurality of personalized glucose targets includes one or more of a target glucose range or a target average glucose.

6. The system of claim 5, wherein the personalized target glucose range includes a personalized lower glucose limit.

7. The system of claim 5, wherein the personalized target glucose range includes a personalized upper glucose limit.

8. The system of claim 1, wherein the at least one physiological parameter is selected from the group consisting of: a red blood cell glucose uptake, a red blood cell lifespan, a red blood cell glycation rate constant, a red blood cell generation rate constant, a red blood cell elimination constant, and an apparent glycation constant.

9. The system of claim 8, wherein the plurality of interfaces further includes the at least one physiological parameter for the subject.

10. The system of claim 1, wherein the user type includes a health care professional.

11. The system of claim 10, wherein the plurality of interfaces includes a glucose monitoring data interface, a glycated hemoglobin interface, a personalized a1c interface, a personalized glucose interface, a personalized average glucose, and a personalized time in range interface.

12. The system of claim 1, wherein the user type includes the subject.

13. The system of claim 12, wherein the plurality of interfaces includes a glucose monitoring data interface, a glycated hemoglobin interface, a mean glucose interface, and a time in range interface.

14. The system of claim 1, wherein the plurality of interfaces comprising the report are predetermined based on the user type.

15. The system of claim 1, wherein the plurality of interfaces comprising the report can be selected by the user.

16. The system of claim 4, wherein the at least one processor is further configured to output a notification if at least one of the plurality of personalized glucose metrics is at or above the corresponding plurality of personalized glucose target.

17. The system of claim 16, wherein the notification comprises a visual notification.

18. The system of claim 16, wherein the notification comprises an audio notification.

19. The system of claim 16, wherein the notification is an alarm.

20. The system of claim 16, wherein the notification is a prompt.

21. The system of claim 1, wherein the reader device wirelessly receives the glycated hemoglobin level for the subject from an electronic medical records system.

22. The system of claim 1, wherein the reader device wirelessly receives the glycated hemoglobin level for the subject from a cloud-based database.

23. The system of claim 1, wherein the reader device wirelessly receives the glycated hemoglobin level for the subject from a QR code.

24. The system of claim 1, the reader device wirelessly receives the glycated hemoglobin level for the subject from a home test kit.
